SEQUENCE LISTING (86 sequences)

SEQ ID NO: 1 - protein, 160 aa, Escherichia coli [sequence omitted]

SEQ ID NO: 2 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 3 - protein, 160 aa, synthetic construct
  REGION 1..160: note = Synthetic
IACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 4 - protein, 160 aa, synthetic construct
  REGION 1..160: note = Synthetic
LACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 5 - protein, 160 aa, synthetic construct
  REGION 1..160: note = Synthetic
VACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 6 - protein, 160 aa, synthetic construct
  REGION 1..160: note = Synthetic
MACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 7 - protein, 160 aa, synthetic construct
  REGION 1..160: note = Synthetic
YACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 8 - protein, 160 aa, synthetic construct
  REGION 1..160: note = Synthetic
WACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 9 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 10 - protein, 160 aa, synthetic construct
  REGION 1..160: note = Synthetic
FACKTASGTAIPIGAGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 11 - protein, 160 aa, synthetic construct
  REGION 1..160: note = Synthetic
FACKTASGTAIPIGPGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 12 - protein, 160 aa, synthetic construct
  REGION 1..160: note = Synthetic
FACKTASGTAIPIGGASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 13 - protein, 160 aa, synthetic construct
  REGION 1..160: note = Synthetic
FACKTASGTAIPIGGPSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 14 - protein, 160 aa, synthetic construct
  REGION 1..160: note = Synthetic
FACKTASGTAIPIGAASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 15 - protein, 160 aa, synthetic construct
  REGION 1..160: note = Synthetic
FACKTASGTAIPIGGGSANVYVNLAPVVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 16 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 17 - protein, 160 aa, synthetic construct
  REGION 1..160: note = Synthetic
FACKTASGTAICIGGGSCNVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 18 - protein, 160 aa, synthetic construct
  REGION 1..160: note = Synthetic
FACKTASGTAIPICGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQCVWNIYANNDVVVPTGG

SEQ ID NO: 19 - protein, 160 aa, synthetic construct
  REGION 1..160: note = Synthetic
FACKTASGTAIPIGGGSANVYVNLACVVNVGQNLCVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 20 - protein, 160 aa, synthetic construct
  REGION 1..160: note = Synthetic
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDCVVPTGG

SEQ ID NO: 21 - protein, 160 aa, synthetic construct
  REGION 1..160: note = Synthetic
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVCPTGG

SEQ ID NO: 22 - protein, 160 aa, synthetic construct
  REGION 1..160: note = Synthetic
FACKTASGTAIPIGGGSANVYVNLAPCVNVGQNCVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 23 - protein, 160 aa, synthetic construct
  REGION 1..160: note = Synthetic
FACKTASGTAIPIGGGSANVYVNLAPVCNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 24 - protein, 160 aa, synthetic construct
  REGION 1..160: note = Synthetic
FACKTASGTAIPIGGGSANVYVNLAPVCNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 25 - protein, 160 aa, synthetic construct
  REGION 1..160: note = Synthetic
FACKTASGTAIPIGGGSANVYVNLAPVVNVGCNLVVDLSTQIFCHNDYPETITDYVTLQRGSAYGGVLSSFSGTVKYSGSSYPFPTTSETPRVVYNSRTDKPWPVALCLTPVSSAGGVAIKAGSLIAVLILRQTNNYNSDDFQFVWNIYANNDVVVPTGG

SEQ ID NO: 26 - protein, 160 aa, synthetic construct
  REGION 1..160: note = Synthetic
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 27 - protein, 160 aa, synthetic construct
  REGION 1..160: note = Synthetic
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 28 - protein, 160 aa, synthetic construct
  REGION 1..160: note = Synthetic
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVACYLTPVSSAGGVAIKAGSLIAVLILRQTNNYNSDDFQFVWNIYANNDVVVPTGG

SEQ ID NO: 29 - protein, 160 aa, synthetic construct
  REGION 1..160: note = Synthetic
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 30 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGCAYGGVLSSFSGTVKYSGSSYPFPCTSETPRVVYNSRTDKPWPVALYLTPVSSAGGVAIKAGSLIAVLILRQTNNYNSDDFQFVWNIYANNDVVVPTGG

SEQ ID NO: 31 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGCAYGGVLSSFSGTVKYSGSSYPFPTTSETPRVVYNSRTDKPWPVALYLTPVSSAGGVAIKAGSLIAVCILRQTNNYNSDDFQFVWNIYANNDVVVPTGG

SEQ ID NO: 32 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSACGGVCSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 33 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSACGGVLSSFSGTVKYSGSSYPFPTTSETPRVVYNSRTDKPWPVALYLTPVSSAGGVAIKAGSLICVLILRQTNNYNSDDFQFVWNIYANNDVVVPTGG

SEQ ID NO: 34 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVCSSCSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 35 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPCSSAGGVAIKAGSLIAVLILRQTNNYNSDDFQFVWNIYANNDVVVPCGG

SEQ ID NO: 36 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSACGVAIKAGSLIAVLILRQTNNYNSDDFQFVWNIYANNDVVVPTGG

SEQ ID NO: 37 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSAGGVAIKAGSLIAVLILRQTNNYNSDDFQFVWNIYANNDVVVPCGG

SEQ ID NO: 38 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGCAIKAGSLIAVLILRQTNNYNSDDFQFVWNIYANNDVVCPTGG

SEQ ID NO: 39 - protein, 160 aa, synthetic construct
  REGION 1..160: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVCIKAGSLIAVLILRQTNNYNSDDFQFVWNIYANNDVCVPTGG

SEQ ID NO: 40 - protein, 160 aa, synthetic construct
  REGION 1..160: note = Synthetic
FACKTASGTAIPIGGGSANVYVNLAPAVNVGQNNVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 41 - protein, 160 aa, synthetic construct
  REGION 1..160: note = Synthetic
FACKTASGTAIPIGGGSANVYVNLAPAVNVGQNSVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 42 - protein, 160 aa, synthetic construct
  REGION 1..160: note = Synthetic
FACKTASGTAIPIGGGSANVYVNLAPAVNVGQNTVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 43 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVNIKAGSLIAVLILRQTNNYNSDDFQFVWNIYANNDVVVPTGG

SEQ ID NO: 44 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVSIKAGSLIAVLILRQTNNYNSDDFQFVWNIYANNDVVVPTGG

SEQ ID NO: 45 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVTIKAGSLIAVLILRQTNNYNSDDFQFVWNIYANNDVVVPTGG

SEQ ID NO: 46 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSVGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 47 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDISARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 48 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTIYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 49 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADITITVNGKVVAK

SEQ ID NO: 50 - protein, 160 aa, synthetic construct
  REGION 1..160: note = Synthetic
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 51 - protein, 160 aa, synthetic construct
  REGION 1..160: note = Synthetic
FACKTASGTAIPIGGASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 52 - protein, 160 aa, synthetic construct
  REGION 1..160: note = Synthetic
FACKTASGTAIPIGPGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 53 - protein, 160 aa, synthetic construct
  REGION 1..160: note = Synthetic
FACKTASGTAIPIGGPSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 54 - protein, 160 aa, synthetic construct
  REGION 1..160: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 55 - protein, 160 aa, synthetic construct
  REGION 1..160: note = Synthetic
FACKTASGTAIPIGGGSANVYVNLAPAVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 56 - protein, 160 aa, synthetic construct; identical to SEQ ID NO: 2
  REGION 1..160: note = Synthetic

SEQ ID NO: 57 - protein, 160 aa, synthetic construct
  REGION 1..160: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 58 - protein, 160 aa, synthetic construct
  REGION 1..160: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 59 - protein, 300 aa, synthetic construct
  REGION 1..300: note = Synthetic
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 60 - protein, 300 aa, synthetic construct
  REGION 1..300: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 61 - protein, 300 aa, synthetic construct
  REGION 1..300: note = Synthetic
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 62 - protein, 300 aa, synthetic construct
  REGION 1..300: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 63 - protein, 300 aa, synthetic construct
  REGION 1..300: note = Synthetic
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 64 - protein, 300 aa, synthetic construct
  REGION 1..300: note = Synthetic
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 65 - protein, 19 aa, Mus musculus
ETDTLLLWVLLLWVPGSTG

SEQ ID NO: 66 - DNA, 741 bp, synthetic construct
  misc_feature 1..741: note = Synthetic
  source 1..741: mol_type = other DNA
atggagaccgacacactgctgctgtgggtgcttttgttgtgggtaccagggtccacgggatttgcctgcaagaccgcctctggaactgcgatacccataggggctgcgagtgcgaacgtctatgtcaacctcgcgccagccgtaaatgtaggacaaaatctcgtggttgacttgagtacgcagatcttctgccacaatgactaccctgagactataacggattatgttactctgcagcggggttctgcgtatgggggcgttttgtcttctttttccggtactgtgaaatacagcggttcctcttatccattccctactacgagcgaaactcccagggtcgtttataattcccgcacggacaagccttggccggttgctctttacctcacgcctgttagcagcgcgggcggtgtcgctatcaaggctggtagtctcatcgcggtcttgattttgaggcagactaacaactacaatagtgatgatttccaatttgtgtggaacatttatgcgaataatgacgtagtggttccaacgggaggatcatcaggtggtggagccacacctaataatatgggcttgattgcaggggcggtgggtggcagtctcttggcagcgttggtaatttgcgggattgtctactggatgcgacggcatactcaaaaagcgccaaagcgaattaggctgcctcatatacgggaagacgaccagccctcttcccatcaaccgctgttctactagtaa

SEQ ID NO: 67 - protein, 245 aa, synthetic construct
  REGION 1..245: note = Synthetic
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGSSGGGATPNNMGLIAGAVGGSLLAALVICGIVYWMRRHTQKAPKRIRLPHIREDDQPSSHQPLFY

SEQ ID NO: 68 - DNA, 750 bp, synthetic construct
  misc_feature 1..750: note = Synthetic
  source 1..750: mol_type = other DNA
atggagaccgacacactgctgctgtgggtgcttttgttgtgggttccagggtcaaccgggtttgcgtgcaaaactgcgtcaggaacggcgataccgataggcgctgcatctgcgaatgtctacgtgaatctggcacccgcagtgaacgtaggacagaacttggtcgtagacttgtccacccagatattctgtcacaacgattacccagagaccataacggattacgttacccttcagcgcggctcagcgtacggaggagttctttcatcttttagcggaacggtcaagtatagcggcagctcctaccctttcccaactactagtgagactccaagagtggtatataacagtagaaccgataaaccgtggcccgttgccctctacctgaccccggtgagtagcgctgggggcgttgcaataaaagcagggagccttattgcggttcttatactccgccaaacaaacaactataactcagacgactttcaattcgtttggaacatctacgccaacaacgatgtcgtggtgcccactggaggctcctctggcggcggttacgagcaatatatcaaatggccgtggtatatatggctcggattcatcgcgggattgatagcgatcgttatggtgaccatcatgttgtgttgtatgacttcctgctgttcatgcctcaagggatgttgctcatgcggatcatgctgcaagtttgacgaagacgattccgagcctgttcttaaaggggtgtgatag

SEQ ID NO: 69 - protein, 248 aa, synthetic construct
  REGION 1..248: note = Synthetic
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGSSGGGYEQYIKWPWYIWLGFIAGLIAIVMVTIMLCCMTSCCSCLKGCCSCGSCCKFDEDDSEPVLKGV

SEQ ID NO: 70 - DNA, 672 bp, synthetic construct
  misc_feature 1..672: note = Synthetic
  source 1..672: mol_type = other DNA
atggagaccgacacactgctgctgtgggtgcttttgttgtgggtgcccggttctaccggatttgcgtgtaagaccgcctctgggactgcgatacccatcggtgctgcatccgctaacgtgtatgtgaatctcgccccagccgtcaacgtaggtcaaaacttggttgttgacttgtctacgcagatattttgtcacaatgattacccagaaacgattaccgactatgttacactccaacggggcagcgcctatggcggtgtactcagcagtttcagtggtacagtgaaatattctggcagcagttatccatttcccacaactagcgaaacccctagagttgtatataactcacgaacggacaagccttggccggtggcgctctatctgaccccggttagctcagcagggggagtggcaattaaggcggggagtttgatcgccgtgcttatactgcgccaaaccaacaattacaatagtgacgattttcaatttgtctggaacatatacgccaataacgacgtcgttgtgccaactggaggtagttctggtggcggtcccaacaaagggagtggaacaacatccgggactacgcgattgctttccggccatacttgctttacactgactggcctgttggggacgctcgttacgatgggtctgctcacctgatga

SEQ ID NO: 71 - protein, 222 aa, synthetic construct
  REGION 1..222: note = Synthetic
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGSSGGGPNKGSGTTSGTTRLLSGHTCFTLTGLLGTLVTMGLLT

SEQ ID NO: 72 - DNA, 966 bp, synthetic construct
  misc_feature 1..966: note = Synthetic
  source 1..966: mol_type = other DNA
atggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatga

SEQ ID NO: 73 - protein, 320 aa, synthetic construct
  REGION 1..320: note = Synthetic
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQGGSSGGGADVTITVNGKVVAK

SEQ ID NO: 74 - protein, 7 aa, synthetic construct
  REGION 1..7: note = Synthetic
GGSSGGG

SEQ ID NO: 75 - RNA, 52 nt, synthetic construct
  misc_feature 1..52: note = Synthetic
  source 1..52: mol_type = other RNA
gaataaactagtattcttctggtccccacagactcagagagaacccgccacc

SEQ ID NO: 76 - RNA, 295 nt, synthetic construct
  misc_feature 1..295: note = Synthetic
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 77 - RNA, 52 nt, synthetic construct
  source 1..52: mol_type = other RNA
  modified_base 1..42: mod_base = m1f
gaanaaacnagnanncnncnggnccccacagacncagagagaacccgccacc

SEQ ID NO: 78 - RNA, 295 nt, synthetic construct
  source 1..295: mol_type = other RNA
  modified_base 1..295: mod_base = m1f
cncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacacccnggagcnagc

SEQ ID NO: 79 - protein, 1273 aa, SARS-CoV2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 80 - protein, 5 aa, SARS-CoV2
KLHYT

SEQ ID NO: 81 - protein, 8 aa, SARS-CoV2
KFDEDDSE

SEQ ID NO: 82 - RNA, 741 nt, synthetic construct
  source 1..741: mol_type = other RNA
atggagaccgacacactgctgctgtgggtgcttttgttgtgggtaccagggtccacgggatttgcctgcaagaccgcctctggaactgcgatacccataggggctgcgagtgcgaacgtctatgtcaacctcgcgccagccgtaaatgtaggacaaaatctcgtggttgacttgagtacgcagatcttctgccacaatgactaccctgagactataacggattatgttactctgcagcggggttctgcgtatgggggcgttttgtcttctttttccggtactgtgaaatacagcggttcctcttatccattccctactacgagcgaaactcccagggtcgtttataattcccgcacggacaagccttggccggttgctctttacctcacgcctgttagcagcgcgggcggtgtcgctatcaaggctggtagtctcatcgcggtcttgattttgaggcagactaacaactacaatagtgatgatttccaatttgtgtggaacatttatgcgaataatgacgtagtggttccaacgggaggatcatcaggtggtggagccacacctaataatatgggcttgattgcaggggcggtgggtggcagtctcttggcagcgttggtaatttgcgggattgtctactggatgcgacggcatactcaaaaagcgccaaagcgaattaggctgcctcatatacgggaagacgaccagccctcttcccatcaaccgctgttctactagtaa

SEQ ID NO: 83 - RNA, 750 nt, synthetic construct
  source 1..750: mol_type = other RNA
atggagaccgacacactgctgctgtgggtgcttttgttgtgggttccagggtcaaccgggtttgcgtgcaaaactgcgtcaggaacggcgataccgataggcgctgcatctgcgaatgtctacgtgaatctggcacccgcagtgaacgtaggacagaacttggtcgtagacttgtccacccagatattctgtcacaacgattacccagagaccataacggattacgttacccttcagcgcggctcagcgtacggaggagttctttcatcttttagcggaacggtcaagtatagcggcagctcctaccctttcccaactactagtgagactccaagagtggtatataacagtagaaccgataaaccgtggcccgttgccctctacctgaccccggtgagtagcgctgggggcgttgcaataaaagcagggagccttattgcggttcttatactccgccaaacaaacaactataactcagacgactttcaattcgtttggaacatctacgccaacaacgatgtcgtggtgcccactggaggctcctctggcggcggttacgagcaatatatcaaatggccgtggtatatatggctcggattcatcgcgggattgatagcgatcgttatggtgaccatcatgttgtgttgtatgacttcctgctgttcatgcctcaagggatgttgctcatgcggatcatgctgcaagtttgacgaagacgattccgagcctgttcttaaaggggtgtgatag

SEQ ID NO: 84 - RNA, 672 nt, synthetic construct
  source 1..672: mol_type = other RNA
atggagaccgacacactgctgctgtgggtgcttttgttgtgggtgcccggttctaccggatttgcgtgtaagaccgcctctgggactgcgatacccatcggtgctgcatccgctaacgtgtatgtgaatctcgccccagccgtcaacgtaggtcaaaacttggttgttgacttgtctacgcagatattttgtcacaatgattacccagaaacgattaccgactatgttacactccaacggggcagcgcctatggcggtgtactcagcagtttcagtggtacagtgaaatattctggcagcagttatccatttcccacaactagcgaaacccctagagttgtatataactcacgaacggacaagccttggccggtggcgctctatctgaccccggttagctcagcagggggagtggcaattaaggcggggagtttgatcgccgtgcttatactgcgccaaaccaacaattacaatagtgacgattttcaatttgtctggaacatatacgccaataacgacgtcgttgtgccaactggaggtagttctggtggcggtcccaacaaagggagtggaacaacatccgggactacgcgattgctttccggccatacttgctttacactgactggcctgttggggacgctcgttacgatgggtctgctcacctgatga

SEQ ID NO: 85 - RNA, 966 nt, synthetic construct
  source 1..966: mol_type = other RNA
atggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatga

SEQ ID NO: 86 - DNA, 80 bp, synthetic construct
  source 1..80: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0256900 A1
(43) Pub. Date: Sep. 3, 2026

(54) POLYNUCLEOTIDE COMPOSITIONS AND USES THEREOF

(71) Applicant: Pfizer Inc., New York, NY (US)

(72) Inventors: Ye Che, Niantic, CT (US); Laurent Olivier Chorro, New York, NY (US); Fernando Martin Diaz, Boston, MA (US); Robert George Konrad Donald, South Orange, NJ (US); Jin Li, Winchester, MA (US); Natalie Clare Silmon de Monerri, New York, NY (US); Raphael Simon, Upper Saddle River, NJ (US)

(21) Appl. No.: 18/718,479

(22) PCT Filed: Dec. 14, 2022
(86) PCT No.: PCT/IB2022/062232

§ 371 Date: Jun. 11, 2024

Related U.S. Application Data
(60) Provisional application No. 63/290,895, filed on Dec. 17, 2021.
(60) Provisional application No. 63/384,607, filed on Nov. 22, 2022.

(51) Int. Cl. A61K 39/108; A61K 39/00; A61P 37/04; C07K 14/245

(52) U.S. Cl. CPC A61K 39/0258; A61P 37/04; C07K 14/245; A61K 2039/53

(57) ABSTRACT

The invention relates to RNA molecules encoding an E. coli fimbrial H antigen (FimH). The present disclosure further relates to compositions comprising the RNA molecules formulated in a lipid nanoparticle (RNA-LNP). The present disclosure further relates to the use of the RNA 5 molecules, RNA-LNPs and compositions for the prevention of E. coli infection, including urinary tract infection.

### RELATED APPLICATIONS

This application claims priority to U.S. Provisional Application No. 63/290,895 filed Dec. 17, 2021 and U.S. Provisional Application No. 63/384,607 filed Nov. 22, 2022. The entire content of each of the foregoing applications is herein incorporated by reference in its entirety.

### FIELD OF THE INVENTION

The invention relates to compositions and methods for the preparation, manufacture and therapeutic use of ribonucleic acid vaccines comprising polynucleotide molecules encoding one or more Escherichia coli (E. coli) fimbrial antigens, such as fimbrial H antigen (FimH).

### REFERENCE TO SEQUENCE LISTING

This application is being filed electronically via EFS-Web and includes an electronically submitted sequence listing in .txt format. The .txt file contains a sequence listing entitled “PC72808-PRV2 Sequence Listing.xml” created on Nov. 21, 2022 and having a size of 121 KB. The sequence listing contained in this .xml file is part of the specification and is incorporated herein by reference in its entirety.

### BACKGROUND OF THE INVENTION

Urinary tract infections (UTI) affect 1 in 5 women at least once during their lifetime and are responsible for significant morbidity and mortality, resulting in a substantial burden on healthcare systems. While several different bacteria can cause UTI, the most common cause (90-95% of cases) is the Gram-negative bacteria Escherichia coli (E. coli). Most E. coli UTI are caused by uropathogenic E. coli (UPEC) that colonise the gastrointestinal tract and migrate from the faecal flora to the urogenital tract, where they adhere to host uroepithelial cells, thus establishing a reservoir for ascending infections of the urinary tract. Adhesion is facilitated by fimbrial adhesins including type 1 fimbriae, which bind to mannosylated glycoproteins in the epithelial layer or secreted into the urine. Type 1 fimbriae are highly conserved among clinical UPEC isolates and are encoded by a cluster of genes called fim, which encode accessory proteins (FimC, FimD), various structural subunits (FimE, FimF, FimG) and an adhesin called FimH. FimH is essential for all characteristics of UTI infection in mouse models that mimic aspects of human bladder infection (Hannan et al. PLoS Pathog. 2010 Aug. 12; 6(8):e1001042; doi: 10.1371/journal.ppat.1001042; Schwartz et al. Infect Immun. 2011 October; 79(10):4250-9. doi: 10.1128/IAI.05339-11). Small molecule inhibitors that target FimH by mimicking mannosylated receptors further validate the role of FimH in UTI, and are showing promise as therapeutics in animal models (Cusumano C K. et al. Sci Transi Med. 2011; 3(109):109ra115. doi:10.1126/scitranslmed.3003021). In addition, FimH is under positive selection in E. coli human cystitis isolates (Chen S L, et al. Proc Natl Acad Sci USA. 2009 Dec. 29; 106(52):22439-44. doi: 10.1073/pnas.0902179106) and positively selected residues may influence virulence in mouse models of cystitis (Schwartz, D. J. et al. Proc Natl Acad Sci USA 110, 15530-15537, doi:10.1073/pnas.1315203110 (2013)).

FimH is composed of two domains, the lectin binding domain (FimH_{LD}) responsible for binding to mannosylated glycoproteins, and the pilin domain. The pilin domain serves to link FimH to other structural subunits of the pilus such as FimG, via a mechanism called donor strand exchange (Le Trong, I et al., J. Struct Biol. 2010 December; 172(3):380-8. doi: 10.1016/j.jsb.2010.06.002). The FimH pilin domain forms an incomplete immunoglobulin fold, resulting in a groove that provides a binding site for the N-terminal 3-strand of FimG, forming a strong intermolecular linkage between FimH and FimG. While FimH_{LD} can be expressed in a soluble, stable form, full length FimH is unstable alone (Vetsch, M., et al. J. Mot. Biol. 322:827-840 (2002); Barnhart M M, et al. Proc Nat/Acad Sci USA. (2000) July 5; 97(14):7709-14) unless in a complex with the chaperone FimC or complemented with the donor strand peptide of FimG in peptide form or as a fusion protein (Barnhart M M, et al., Proc Natl Acad Sci USA. (2000) July 5; 97(14):7709-14; Sauer M M, et al. Nat Commun. (2016) March 7; 7:10738; Barnhart M M, et al. J Bacteriol. 2003 May; 185(9):2723-30). The design and expression of a full length FimH molecule by linking the FimG donor peptide to full length FimH via a glycine-serine linker has been previously described (PCT Intl. Publication No. WO2021/084429, published May 6, 2021), and is designated FimH-DSG.

FimH_{LD} is thought to be a poor immunogen in terms of its ability to stimulate functional immunogenicity. Some studies suggest that although binding antibody titers can be elicited with FimH_{LD} with and without adjuvant, functional neutralizing titers were only observed in the presence of adjuvant (PCT Intl. Publication No. WO2021/084429, published May 6, 2021). Studies suggest that locking FimH in an open conformation, with reduced affinity for mannoside ligands, improves functional immunogenicity (Kisiela, D. I. et al., Proc Natl Acad Sci USA 110, 19089-19094 (2013).

Accordingly, there is a need for improved immunogenic compositions comprising FimH antigens with reduced affinity for mannoside ligands and improved biochemical properties that result in improved functional immunogenicity relative to wild type FimH.

### SUMMARY OF THE INVENTION

The present disclosure provides the unmet need for improved immunogenic compositions against E. coli infection, among other things, as provided herein.

In one aspect, the disclosure provides immunogenic compositions and methods for preventing, treating or ameliorating an infection, disease or condition in a subject comprising the administration of RNA molecules, e.g., immunogenic RNA polynucleotide encoding an amino acid sequence, e.g., an immunogenic antigen, comprising an E. coli FimH protein (“FimH”), an immunogenic variant thereof, or an immunogenic fragment of the FimH protein or the immunogenic variant thereof, e.g., an antigenic peptide or protein. Thus, the immunogenic antigen comprises an epitope of a FimH protein for inducing an immune response against FimH, in the subject. RNA polynucleotide encoding an immunogenic antigen is administered to provide (following expression of the polynucleotide by appropriate target cells) antigen for induction, e.g., stimulation, priming, and/or expansion, of an immune response, e.g., antibodies and/or immune effector cells. In one aspect, the immune response to be induced according to the present disclosure is both B cell-mediated immune response, e.g., an antibody-mediated immune response as well as T-cell-mediated immune response. In one aspect, the immune response is an anti-FimH immune response.

The immunogenic compositions described herein comprise RNA molecules comprising RNA (as the active principle) that may be translated into one or more proteins in a recipient's cells. In addition to wild type, codon-optimized or mutant sequences encoding the antigen sequence, the RNA molecules may contain one or more structural elements optimized for maximal efficacy of the RNA with respect to stability and translational efficiency (5′ cap, 5′ UTR, subgenomic promoter, 3′ UTR, poly-A-tail). In one aspect, the RNA molecules contain all of these elements. The RNA molecules described herein may be complexed with lipids and/or proteins to generate RNA-particles (e.g., lipid nanoparticles (LNPs)) for administration. In one aspect, the RNA molecules described herein are complexed with lipids to generate RNA-lipid nanoparticles (e.g. RNA-LNPs) for administration. In one aspect, the RNA molecules described herein are complexed with proteins for administration. In one aspect, the RNA molecules described herein are complexed with lipids and proteins for administration. If a combination of different RNA molecules is used, the RNA molecules may be complexed together or complexed separately with lipids and/or proteins to generate RNA-particles for administration.

The present disclosure provides for RNA molecules and RNA-LNPs that include at least one open reading frame (ORF) encoding a FimH antigen. In some aspects, the FimH antigen is a FimH polypeptide. In some aspects, the FimH polypeptide is a full-length, truncated, fragment or variant thereof. In some aspects, the FimH polypeptide comprises at least one mutation.

The present disclosure provides for RNA molecules and RNA-LNPs that include at least one ORF encoding a FimH polypeptide of Table 1. In some aspects, the FimH polypeptide comprises an amino acid sequence selected from SEQ ID NO: 67, 69, 71 or 73. In some aspects, the FimH polypeptide has, has at least, or has at most 90%, 91%, 92%, 93%, 94%, 95, 96%, 97%, 98% or 99% or higher identity to any of the amino acid sequences of Table 1, for example, any of SEQ ID NO: 67, 69, 71 or 73. In some aspects, the FimH polypeptide consists of any of the amino acid sequences of Table 1, for example, any of SEQ ID NO: 67, 69, 71 or 73.

In another aspect, the present disclosure provides for RNA molecules and RNA-LNPs that include at least one ORF encoding a FimH polypeptide wherein the FimH polypeptide is FimH-DSG (SEQ ID NO: 59), FimH-DSG triple mutant (G15A, G16A, V27A) (SEQ ID NO: 62), FimHLD triple mutant (G15A, G16A, V27A) (SEQ ID NO: 54), an immunogenic fragment thereof, or a combination of any two or more of the foregoing. In some aspects, the FimH polypeptide has, has at least, or has at most 90%, 91%, 92%, 93%, 94%, 95, 96%, 97%, 98% or 99% or higher identity to any of the amino acid sequences of SEQ ID NO: 59, 62 or 54.

The present disclosure provides for RNA molecules and RNA-LNPs comprising at least one ORF transcribed from at least one DNA nucleic acid of Table 2. In some aspects, the RNA molecule is transcribed from a nucleic acid sequence selected from SEQ ID NO: 66, 68, 70 or 72. In some aspects, the RNA molecule comprises an ORF transcribed from a nucleic acid sequence that has, has at least, or has at most 90%, 91%, 92%, 93%, 94%, 95, 96%, 97%, 98% or 99% or higher identity to any of the nucleic acid sequences of Table 2, for example, any of SEQ ID NO: 66, 68, 70 or 72. In some aspects, the RNA molecule comprises an ORF transcribed from a nucleic acid sequence that consists of any of the nucleic acid sequences of Table 2, for example, any of SEQ ID NO: 66, 68, 70 or 72.

The present disclosure further provides for RNA molecules and RNA-LNPs comprising at least one ORF comprising an RNA nucleic acid sequence of Table 3. In some aspects, the RNA molecule comprises a nucleic acid sequence selected from SEQ ID NO: 82 to 85. In some aspects, the RNA molecule comprises a nucleic acid sequence that has, has at least, or has at most 90%, 91%, 92%, 93%, 94%, 95, 96%, 97%, 98% or 99% identity to any of the nucleic acid sequences of Table 3, for example, any of SEQ ID NO: 82 to 85. In some aspects, the RNA molecule comprises a nucleic acid sequence that consists of any of the nucleic acid sequences of Table 3, for example, any of SEQ ID NO: 82 to 85. In some aspects, each uridine of any of SEQ ID NO: 82 to 85 is replaced by 1-methyl-3′-pseudouridylyl (4P).

The present disclosure further provides for RNA molecules and RNA-LNPs that include a 5′ untranslated region (5′-UTR) and/or a 3′ untranslated region (3′-UTR). In some aspects, the RNA molecule includes a 5′ untranslated region (5′-UTR). In some aspects, the 5′ UTR comprises a sequence selected from any of SEQ ID NO: 75 or 77. In some aspects, the 5′ UTR comprises a sequence having at least 90%, 91%, 92%, 93%, 94%, 95, 96%, 97%, 98% or 99% or higher identity to any of SEQ ID NO: 75 or 77. In some aspects, the 5′ UTR comprises a sequence selected from any of SEQ ID NO: 75 or 77. In some aspects, the 5′ UTR comprises a sequence consisting of any of SEQ ID NO: 75 or 77.

In some aspects, the RNA molecules and RNA-LNPs include a 3′ untranslated region (3′-UTR). In some aspects, the 3′ UTR comprises a sequence selected from any of SEQ ID NO: 76 or 78. In some aspects, the 3′ UTR comprises a sequence having at least 90%, 91%, 92%, 93%, 94%, 95, 96%, 97%, 98% or 99% or higher identity to any of SEQ ID NO: 76 or 78. In some aspects, the 3′ UTR comprises a sequence selected from any of SEQ ID NO: 76 or 78. In some aspects, the 3′ UTR comprises a sequence consisting of any of SEQ ID NO: 76 or 78.

The present disclosure further provides for RNA molecules and RNA-LNPs that include a 5′ cap moiety. The present disclosure further provides for RNA molecules and RNA-LNPs that include a 3′ poly-A tail. In some aspects, the poly-A tail comprises a sequence having SEQ ID NO: 86.

In some aspects, the RNA molecule includes a 5′ UTR and 3′ UTR. In some aspects, the RNA molecule includes a 5′ cap, 5′ UTR, and 3′ UTR. In some aspects, the RNA molecule includes a 5′ cap, 5′ UTR, 3′ UTR, and poly-A tail. In some aspects, the RNA molecule includes a 5′ cap, 3′ UTR, and poly-A tail.

In some aspects, the poly-A tail length may contain +1/−1 A. In some aspects, the uridine is 1-methyl-3′-pseudouridylyl (P).

The present disclosure further provides for RNA molecules that include at least one open reading frame that is codon-optimized. The present disclosure further provides RNA molecules comprising stabilized RNA. The present disclosure further provides for RNA molecules that include RNA having at least one modified nucleotide. In some aspects, the modified nucleotide is pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyl uridine. In some aspects, the modified nucleotide is 1-methyl-3′-pseudouridylyl (P).

The present disclosure further provides for RNA molecules that are messenger-RNA (mRNA), which can be nucleoside-modified RNA (modRNA). In some aspects, the RNA is a mRNA. In other aspects, the RNA is a modRNA.

The present disclosure further provides for immunogenic compositions including the RNA molecules described herein. The RNA molecules may be formulated in, encapsulated in, complex with, bound to or adsorbed on a lipid nanoparticle (LNP) (e.g., FimH RNA-LNPs) in such immunogenic compositions. In some aspects, lipid nanoparticle includes at least one of a cationic lipid, a PEG-lipid, and at least one structural lipid (e.g., a neutral lipid and a steroid or steroid analog).

In some aspects, lipid nanoparticle includes a cationic lipid. In some aspects, the cationic lipid is (4-hydroxybutyl)azanediyl)bis(hexane-6,1-diyl)bis(2-hexyldecanoate) (ALC-0315).

In some aspects, lipid nanoparticle includes a polymer conjugated lipid. In some aspects, lipid nanoparticle includes a PEG-lipid, also referred to PEGylated lipid. In some aspects, the PEG-lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG, N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. In some aspects, the PEG-lipid is 2-[(polyethylene glycol)-2000]-N,N-ditetradecylacetamide (ALC-0159).

In some aspects, lipid nanoparticle includes at least one structural lipid, such as a neutral lipid. In some aspects, the neutral lipid is selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and/or 1,2-dielaidoyl-sn-glycero-3-phophoethanolamine (transDOPE). In some aspects, the neutral lipid is 1,2-distearoyl-sn-glycero-3-phosphocholine (DSPC).

In some aspects, the lipid nanoparticle includes a second structural lipid, such as a steroid or steroid analog. In some aspects, the steroid or steroid analog is cholesterol.

In some aspects, the lipid nanoparticle has a mean diameter of about 1 to about 500 nm.

In some aspects, the RNA-LNP immunogenic composition is a liquid RNA-LNP composition comprising a RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of a cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In some aspects, the liquid RNA-LNP immunogenic composition comprises a RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the RNA-LNP immunogenic composition is a lyophilized RNA-LNP composition comprising a RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent. Concentrations in the lyophilized RNA-LNP composition are determined post-reconstitution.

In specific aspects, a lyophilized RNA-LNP composition comprises a RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline. Concentrations in the lyophilized RNA-LNP composition are determined post-reconstitution.

The present disclosure provides for RNA molecules, RNA-LNPs and immunogenic compositions that may be administered to a subject at a dose of at least, at most, exactly, or between any two of 1 μg, 15 μg, 30 μg, 45 μg, 60 μg, 75 μg, or 90 μg of FimH RNA encapsulated in LNP.

The present disclosure provides for RNA molecules, RNA-LNPs and immunogenic compositions that may be administered in a single dose. The present disclosure further provides for RNA molecules, RNA-LNPs and immunogenic compositions that may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 1 month later, Day 0 and 2 months later, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later). The present disclosure further provides for RNA molecules, RNA-LNPs and immunogenic compositions that may be administered twice at Day 0 and 2 months later. The present disclosure further provides for RNA molecules, RNA-LNPs and immunogenic compositions that may be administered twice at Day 0 and 6 months later. The present disclosure further provides for RNA molecules, RNA-LNPs and immunogenic compositions that may be administered three, four, five, six, seven, eight, nine, ten, eleven, twelve, thirteen, fourteen, or more administrations. In some aspects, periodic boosters at intervals of 1-5 years may be desirable to maintain protective levels of the antibodies.

The present disclosure provides for a method of inducing an immune response in a subject, including administering to the subject an effective amount of an RNA molecule, RNA-LNP and/or immunogenic composition described herein. The present disclosure further provides for the use of an RNA molecule, RNA-LNP and/or immunogenic composition described herein in the manufacture of a medicament for use in inducing an immune response in a subject.

The present disclosure provides for a method of inducing an immune response in a subject, including administering to the subject an effective amount of an RNA molecule and/or RNA-LNP that includes at least one open reading frame encoding a FimH polypeptide or composition described herein. The present disclosure further provides for the use of an RNA molecule and/or RNA-LNP that includes at least one open reading frame encoding a FimH polypeptide or composition described herein in the manufacture of a medicament for use in inducing an immune response in a subject.

The present disclosure provides for a method of inducing an immune response in a subject, including administering to the subject an effective amount of an RNA molecule and/or RNA-LNP that includes at least one open reading frame encoding a polypeptide of a gene of interest or composition described herein. The present disclosure further provides for the use of an RNA molecule and/or RNA-LNP that includes at least one open reading frame encoding a polypeptide of a gene of interest or composition described herein in the manufacture of a medicament for use in inducing an immune response in a subject.

The present disclosure provides for a method of preventing, treating or ameliorating an infection, disease or condition in a subject, including administering to a subject an effective amount of an RNA molecule, RNA-LNP and/or immunogenic composition described herein. The present disclosure further provides for the use of an RNA molecule RNA-LNP and/or immunogenic composition described herein in the manufacture of a medicament for use in preventing, treating or ameliorating an infection, disease or condition in a subject. In some aspects, the infection or condition is associated with E. coli FimH. In some aspects, the infection, disease or condition is a urinary tract infection (UTI), urosepsis, cystitis or pyelonephritis.

The present disclosure provides for a method of preventing, treating or ameliorating an infection, disease or condition in a subject, including administering to a subject an effective amount of an RNA molecule and/or RNA-LNP that includes at least one open reading frame encoding a FimH polypeptide or immunogenic composition described herein. The present disclosure further provides for the use of an RNA molecule and/or RNA-LNP that includes at least one open reading frame encoding a FimH polypeptide or immunogenic composition described herein in the manufacture of a medicament for use in preventing, treating or ameliorating an infection, disease or condition in a subject. In some aspects, the infection, disease or condition is associated with E. coli FimH. In some aspects, the infection, disease or condition is urinary tract infection (UTI), urosepsis, cystitis or pyelonephritis.

The present disclosure further provides for a method of preventing, treating or ameliorating an infection, disease or condition in a subject, including administering to a subject an effective amount of RNA molecules and/or RNA-LNPs that include at least one open reading frame encoding a polypeptide of a gene of interest or immunogenic compositions described herein. The present disclosure further provides for the use of RNA molecules and/or RNA-LNPs that include at least one open reading frame encoding a polypeptide of a gene of interest or immunogenic compositions described herein in the manufacture of a medicament for use in preventing, treating or ameliorating an infection, disease or condition in a subject. In some aspects, the infection, disease or condition is associated with the gene of interest.

In some aspects, the subject is, is at least, or is at most less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject the subject is about 50 years of age or older. In a further aspect, the subject is between 6 months and 1 year old, 1 year old to 2 year old, 1 year old to 3 year old, 1 year old to 4 year old, 1 year old to 5 year old, 6 months old to 5 years old, or 60 years of age or older. The entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, and subjects greater than 50 years of age.

In some embodiments, the subject is a human. In some particular embodiments, the human is a child, such as an infant. In some other particular embodiments, the human is a woman, particularly a pregnant woman. In some aspects, the subject is immunocompetent. In some aspects, the subject is immunocompromised.

The present disclosure provides for a method or use described herein, wherein the RNA molecule, RNA-LNP and/or immunogenic composition is administered as a vaccine.

The present disclosure provides a method or use described herein, wherein the RNA molecule, RNA-LNP and/or immunogenic composition is administered by intradermal or intramuscular injection.

One embodiment of the invention provides an E. coli vaccine comprising: at least one ribonucleic acid polynucleotide having an open reading frame encoding at least one FimH antigenic polypeptide (RNA) or an immunogenic fragment thereof, formulated in a lipid nanoparticle.

In one aspect of the E. coli vaccine, the RNA further comprises a 5′ cap analog. In a preferred aspect, the 5′ cap analog comprises m7G(5′)ppp(5′)(2′OMeA)pG.

In another aspect of the E. coli vaccine, the RNA further comprises a modified nucleotide.

In another aspect of the E. coli vaccine, wherein the at least one antigenic polypeptide is FimH-DSG (SEQ ID NO: 59), FimH-DSG triple mutant (G15A, G16A, V27A) (SEQ ID NO: 62), FimHLD triple mutant (G15A, G16A, V27A) (SEQ ID NO: 54), an immunogenic fragment thereof, or a combination of any two or more of the foregoing.

In another aspect of the E. coli vaccine, wherein the vaccine comprises a) at least one ribonucleic acid (RNA) polynucleotide having an open reading frame encoding FimH-DSG (SEQ ID NO: 59); b) at least one ribonucleic acid (RNA) polynucleotide having an open reading frame encoding FimH-DSG triple mutant (G15A, G16A, V27A) (SEQ ID NO: 62); or c) at least one ribonucleic acid (RNA) polynucleotide having an open reading frame encoding FimHLD triple mutant (G15A, G16A, V27A) (SEQ ID NO: 54).

In another aspect of the E. coli vaccine, wherein the RNA encodes FimH fused to a C-terminal membrane targeting domain.

In another aspect of the E. coli vaccine, wherein the RNA encodes FimH fused to a C-terminal membrane targeting domain and they are separated by a linker. In preferred aspect of the E. coli vaccine, wherein the encoded linker has the amino acid sequence GGSSGGG (SEQ ID NO: 74).

In another aspect of the E. coli vaccine, wherein the C-terminal membrane targeting domain is derived from a viral glycoprotein. In another aspect of the E. coli vaccine, wherein the membrane targeting sequence is derived from HSV gD, SARS-CoV2 Spike protein, or human DAF protein GPI sequence, or a synthetic GPI sequence.

In another aspect of the E. coli vaccine, wherein FimH is secreted and has no C-terminal membrane targeting domain.

In another aspect of the E. coli vaccine, wherein the open reading frame encoded by the RNA is codon-optimized.

In another aspect of the E. coli vaccine, wherein the vaccine further comprises a cationic lipid.

In another aspect of the E. coli vaccine, wherein the vaccine comprises a lipid nanoparticle encompassing the RNA molecule.

In another aspect of the E. coli vaccine, wherein the vaccine comprises a) a lipid nanoparticle encompassing at least one ribonucleic acid (RNA) polynucleotide having an open reading frame encoding FimH-DSG; b) a lipid nanoparticle encompassing at least one ribonucleic acid (RNA) polynucleotide having an open reading frame encoding FimH-DSG triple mutant (G15A, G16A, V27A); or c) at least one ribonucleic acid (RNA) polynucleotide having an open reading frame encoding FimHLD triple mutant (G15A, G16A, V27A) (SEQ ID NO: 54).

In another aspect of the E. coli vaccine, wherein the lipid nanoparticle size is at least 40 nm. In another aspect of the E. coli vaccine, wherein the lipid nanoparticle size is at most 180 nm.

In another aspect of the E. coli vaccine, wherein at least 80% of the total RNA in the composition is encapsulated.

In another aspect of the E. coli vaccine, wherein the vaccine comprises ALC-0315 (4-hydroxybutyl)azanediyl)bis(hexane-6,1-diyl)bis(2-hexyldecanoate).

In another aspect of the E. coli vaccine, wherein the vaccine comprises ALC-0159 (2-[(polyethylene glycol)-2000]-N,N-ditetradecylacetamide).

In another aspect of the E. coli vaccine, wherein the vaccine comprises 1,2-Distearoyl-sn-glycero-3-phosphocholine (DSPC).

In another aspect of the E. coli vaccine, wherein the RNA polynucleotide comprises a 5′ cap, 5′ UTR, 3′ UTR, and polyA tail.

In another aspect of the E. coli vaccine, wherein the 5′ UTR comprises the sequence

(SEQ ID NO: 77)
GAAΨAAACΨAGΨAΨΨCΨΨCΨGGΨCCCCACAGACΨCAGAGAGAACCCGCCA
CC.

In another aspect of the E. coli vaccine, wherein the 5′ UTR comprises the sequence

(SEQ ID NO: 75)
GAAUAAACUAGUAUUCUUCUGGUCCCCACAGACUCAGAGAGAACCCGCCA
CC.

In another aspect of the E. coli vaccine, wherein the 3′ UTR comprises the sequence

(SEQ ID NO: 78)
CΨCGAGCΨGGΨACΨGCAΨGCACGCAAΨGCΨAGCΨGCCCCΨΨΨCCC
GΨCCΨGGGΨACCCCGAGΨCΨCCCCCGACCΨCGGGΨCCCAGGΨAΨG
CΨCCCACCΨCCACCΨGCCCCACΨCACCACCΨCΨGCΨAGΨΨCCAGA
CACCΨCCCAAGCACGCAGCAAΨGCAGCΨCAAAACGCΨΨAGCCΨAG
CCACACCCCCACGGGAAACAGCAGΨGAΨΨAACCΨΨΨAGCAAΨAAA
CGAAAGΨΨΨAACΨAAGCΨAΨACΨAACCCCAGGGΨΨGGΨCAAΨΨCG
ΨGCCAGCCACACCCΨGGAGCΨAGC.

In another aspect of the E. coli vaccine, wherein the 3′ UTR comprises the sequence

(SEQ ID NO: 76)
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCCUGGAGCUAGC.

In another aspect of the E. coli vaccine, wherein P is 1-methyl-3′-pseudouridylyl.

In another aspect of the E. coli vaccine, wherein the poly A tail is 80 nucleotides in length.

In another aspect of the E. coli vaccine, wherein the FimH polypeptide comprises serine substitutions at positions N228 and N235.

All methods described herein can be performed in any suitable order unless otherwise indicated herein or otherwise clearly contradicted by context. The use of any and all examples, or exemplary language (e.g., “such as”), provided herein is intended merely to further illustrate the disclosure and does not pose a limitation on the scope of the claims. No language in the specification should be construed as indicating any non-claimed element essential to the practice of the disclosure.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

It is contemplated that any aspect discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE FIGURES

FIG. 1 describes FimH mRNA constructs. Legend: SP, Mouse IgG Kappa signal peptide; FimH_{LD}, FimH lectin domain; TMD, transmembrane domain; gpi, glycosylphosphatidylinositol anchor.

FimH amino acid substitutions introduced to prevent N-glycosylation (N7S, N70S, N228S and N235S) or to stabilize conformation (G15A, G16A, V27A or triple mutant “TM”) are indicated; G, stabilizing donor strand peptide added to the C-terminus of the full-length FimH protein.

FIG. 2 describes FimH gene and protein sequences of the following constructs: FimH_{LD}TMCt60HSVgD (SEQ ID NO: 66 and SEQ ID NO: 67); FimH_{LD}TMCtΔ5Spike (SEQ ID NO: 68 and SEQ ID NO: 69); FimH_{LD}TMCtDAFgpl (SEQ ID NO: 70 and SEQ ID NO: 71); and Secreted FimH_{DSG}-TM (N-deglycosylated) (SEQ ID NO: 72 and SEQ ID NO: 73). Legend: mouse IgK signal peptides are italicized; Interdomain linkers are underlined; asterisks are stop codons.

FIG. 3 describes immunofluorescent microscopic imaging of FimH surface expression in HeLa cells. Representative confocal microscope images of FimH expressed in HeLa cells 24 h after transfection with 50 ng of the indicated mRNA constructs. FimH was stained with a monoclonal antibody that recognizes an epitope overlapping the mannoside ligand binding site. Only nuclear staining (with DAPI) and little or no FimH expression was observed with mock transfected cells or cells transfected with mRNA encoding the secreted FimH-DSG. Percentages of cells showing FimH surface staining with mRNAs encoding the HSVgD chimera, SARS-COV2ΔCtD5 chimera and DAF-GPI chimera were respectively 36% (8/20), 12% (3/24) and 82% (18/22).

FIGS. 4A and 4B describe FimH expression in transfected HeLa cells. Quantitative analysis of FimH expression in fixed/permeabilized HeLa cells (total FimH) (FIG. 4A) compared with fixed cells (surface FimH) (FIG. 4B). Signal was quantified from individual microplate wells using the Opera confocal microscope platform and Harmony® image analysis software. “MFI”=mean fluorescence intensity.

FIGS. 5A and 5B describe expression of FimH mRNAs in Expi293 cells by flow cytometry using the same monoclonal antibody used for immunofluorescence microscopy. FimH expression detected in permeabilized cells (total FimH; FIG. 5A) and non-permeabilized Expi293 cells (surface FimH only; FIG. 5B). Cell viability exceeded 90% for all mRNA constructs at all concentrations transfected. Baseline MFIs for mock transfected controls for total stained or surface-stained cells are shown as dotted lines (values of 938 and 222, respectively). “MFI”=mean fluorescence intensity.

FIG. 6 describes titers of secreted FimH-DSG-TM in transfected supernatants of Expi293 cells. Levels of secreted his-tagged FimH antigen were detected in the cell culture supernatant by Octet biolayer interferometry.

FIG. 7 describes the flow cytometry gating strategy in Example 2.

FIGS. 8A and 8B describe total and surface FimH expression in transfected human skeletal muscle cells (hSkMCs) showing quantitative analysis of FimH expression in fixed/permeabilized primary hSkMCs (FIG. 8A, total FimH) compared with fixed cells (FIG. 8B, surface FimH). The percentage of cells staining positive for expression of the FimH antigen was determined for each amount of mRNA 24 h after transfection with the indicated LNPs. Signal was quantified from individual microplate wells using the Opera confocal microscope platform and Harmony® image analysis software. Methods are based on the procedure described in Example 1 for HeLa cell transfection.

FIGS. 9A and 9B describe FimH IgG dLIA titers showing neutralizing antibody titers at PD1 and PD2 timepoints following immunization of mice. FIG. 9A shows anti-FimH IgG at week 3 (PD1) and FIG. 9B shows anti-FimH IgG at week 6 (PD2). Responses to 1 μg or 10 μg of the FimH_{DSG} antigen mRNA LNP are significantly greater than responses to FimH_{DSG} protein antigen with LinA-2 adjuvant (****, p<0.0001, ***, p<0.001, **, p<0.01). LLOQ, lower limit of quantitation (0.61 μg/mL) was calculated from standard curve bias. R, responder threshold defined as an FimH IgG serum titer greater than five-fold above LLOQ (3.05 μg/mL).

FIG. 10 describes FimH neutralizing antibody titers at PD1, PD2 and PD3 timepoints. PD2 or PD3 responses to 1 μg of the FimH_{DSG} antigen mRNA LNP are significantly greater than responses to the FimH_{DSG} protein antigen adjuvanted with LiNA-2 (***, p<0.0001). FimH_{DSG} protein antigen was dosed initially at 10 μg followed by two 5 μg booster doses.

FIG. 11 depicts representative flow cytometry dot plots illustrating T-cell gating strategy (top row) and antigen specific stimulation of individual cytokine or surface markers associated with Th1 or Th2 pathways (‘FimH stim’ vs ‘DMSO’). The data in the plots show that FimH peptide stimulation results in the detection of cytokine producing FimH-specific CD4+ and CD8+ T-cells post immunization. ‘FimH stim’ means FimH (peptide) library stimulated.

FIG. 12 depicts that modRNA constructs elicit a stronger CD8+ T cell response compared to protein subunit+LiNA-2, especially at higher concentrations.

FIG. 13A to 13D depict that vaccination with membrane-associated modRNA constructs resulted in an increased frequency of FimH-specific Th1-biased CD4+ T cells compared to FimH_{DSG}(secreted) modRNA and protein subunit+LiNA2.

FIG. 14A to 14C depict that all vaccinations yielded low Th2 and Th17 cytokine-producing FimH specific CD4 T cells after three doses.

### SEQUENCE IDENTIFIERS

SEQ ID NO: 1 sets forth an amino acid sequence for wild type E. coli FimH_{LD} (FimHLD_WT).
SEQ ID NO: 2 sets forth an amino acid sequence for the mutant E. coli FimHLD_G65A_V27A.
SEQ ID NO: 3 sets forth an amino acid sequence for the mutant E. coli FimHLD_F1 I.
SEQ ID NO: 4 sets forth an amino acid sequence for the mutant E. coli FimHLD_F1 L.
SEQ ID NO: 5 sets forth an amino acid sequence for the mutant E. coli FimHLD_F1V.
SEQ ID NO: 6 sets forth an amino acid sequence for the mutant E. coli FimHLD_F1 M.
SEQ ID NO: 7 sets forth an amino acid sequence for the mutant E. coli FimHLD_F1Y.
SEQ ID NO: 8 sets forth an amino acid sequence for the mutant E. coli FimHLD_F1W.
SEQ ID NO: 9 sets forth an amino acid sequence for the mutant E. coli FimHLD_Q133K.
SEQ ID NO: 10 sets forth an amino acid sequence for the mutant E. coli FimHLD_G15A.
SEQ ID NO: 11 sets forth an amino acid sequence for the mutant E. coli FimHLD_G15P.
SEQ ID NO: 12 sets forth an amino acid sequence for the mutant E. coli FimHLD_G16A.
SEQ ID NO: 13 sets forth an amino acid sequence for the mutant E. coli FimHLD_G16P.
SEQ ID NO: 14 sets forth an amino acid sequence for the mutant E. coli FimHLD_G15A_G16A.
SEQ ID NO: 15 sets forth an amino acid sequence for the mutant E. coli FimHLD_R60P.
SEQ ID NO: 16 sets forth an amino acid sequence for the mutant E. coli FimHLD_G65A.
SEQ ID NO: 17 sets forth an amino acid sequence for the mutant E. coli FimHLD_P12C_A18C.
SEQ ID NO: 18 sets forth an amino acid sequence for the mutant E. coli FimHLD_G14C_F144C.
SEQ ID NO: 19 sets forth an amino acid sequence for the mutant E. coli FimHLD_P26C_V35C.
SEQ ID NO: 20 sets forth an amino acid sequence for the mutant E. coli FimHLD_P26C_V154C.
SEQ ID NO: 21 sets forth an amino acid sequence for the mutant E. coli FimHLD_P26C_V156C.
SEQ ID NO: 22 sets forth an amino acid sequence for the mutant E. coli FimHLD_V27C_L34C.
SEQ ID NO: 23 sets forth an amino acid sequence for the mutant E. coli FimHLD_V28C_N33C.
SEQ ID NO: 24 sets forth an amino acid sequence for the mutant E. coli FimHLD_V28C_P157C.
SEQ ID NO: 25 sets forth an amino acid sequence for the mutant E. coli FimHLD_Q32C_Y108C.
SEQ ID NO: 26 sets forth an amino acid sequence for the mutant E. coli FimHLD_N33C_L109C.
SEQ ID NO: 27 sets forth an amino acid sequence for the mutant E. coli FimHLD_N33C_P157C.
SEQ ID NO: 28 sets forth an amino acid sequence for the mutant E. coli FimHLD_V35C_L107C.
SEQ ID NO: 29 sets forth an amino acid sequence for the mutant E. coli FimHLD_V35C_L109C.
SEQ ID NO: 30 sets forth an amino acid sequence for the mutant E. coli FimHLD_S62C_T86C.
SEQ ID NO: 31 sets forth an amino acid sequence for the mutant E. coli FimHLD_S62C_L129C.
SEQ ID NO: 32 sets forth an amino acid sequence for the mutant E. coli FimHLD_Y64C_L68C.
SEQ ID NO: 33 sets forth an amino acid sequence for the mutant E. coli FimHLD_Y64C_A127C.
SEQ ID NO: 34 sets forth an amino acid sequence for the mutant E. coli FimHLD_L68C_F71C.
SEQ ID NO: 35 sets forth an amino acid sequence for the mutant E. coli FimHLD_V112C_T158C.
SEQ ID NO: 36 sets forth an amino acid sequence for the mutant E. coli FimHLD_S113C_G116C.
SEQ ID NO: 37 sets forth an amino acid sequence for the mutant E. coli FimHLD_S113C_T158C.
SEQ ID NO: 38 sets forth an amino acid sequence for the mutant E. coli FimHLD_V118C_V156C.
SEQ ID NO: 39 sets forth an amino acid sequence for the mutant E. coli FimHLD_A119C_V155C.
SEQ ID NO: 40 sets forth an amino acid sequence for the mutant E. coli FimHLD_L34N_V27A.
SEQ ID NO: 41 sets forth an amino acid sequence for the mutant E. coli FimHLD_L34S_V27A.
SEQ ID NO: 42 sets forth an amino acid sequence for the mutant E. coli FimHLD_L34T_V27A.
SEQ ID NO: 43 sets forth an amino acid sequence for the mutant E. coli FimHLD_A119N_V27A.
SEQ ID NO: 44 sets forth an amino acid sequence for the mutant E. coli FimHLD_A119S_V27A.
SEQ ID NO: 45 sets forth an amino acid sequence for the mutant E. coli FimHLD_A119T_V27A.
SEQ ID NO: 46 sets forth an amino acid sequence for the mutant E. coli FimH-DSG_A115V.
SEQ ID NO: 47 sets forth an amino acid sequence for the mutant E. coli FimH-DSG_V1631.
SEQ ID NO: 48 sets forth an amino acid sequence for the mutant E. coli FimH-DSG_V1851.
SEQ ID NO: 49 sets forth an amino acid sequence for the mutant E. coli FimH-DSG_DSG_V31.
SEQ ID NO: 50 sets forth an amino acid sequence for the mutant E. coli FimHLD_G15A_V27A.
SEQ ID NO: 51 sets forth an amino acid sequence for the mutant E. coli FimHLD_G16A_V27A.
SEQ ID NO: 52 sets forth an amino acid sequence for the mutant E. coli FimHLD_G15P_V27A.
SEQ ID NO: 53 sets forth an amino acid sequence for the mutant E. coli FimHLD_G16P_V27A.
SEQ ID NO: 54 sets forth an amino acid sequence for the mutant E. coli FimHLD_G15A_G16A_V27A.
SEQ ID NO: 55 sets forth an amino acid sequence for the mutant E. coli FimHLD_V27A_R60P.
SEQ ID NO: 56 sets forth an amino acid sequence for the mutant E. coli FimHLD_G65A_V27A.
SEQ ID NO: 57 sets forth an amino acid sequence for the mutant E. coli FimHLD_V27A_Q133K.
SEQ ID NO: 58 sets forth an amino acid sequence for the mutant E. coli FimHLD_G15A_G16A_V27A_Q133K.
SEQ ID NO: 59 sets forth an amino acid sequence for wild type E. coli full-length FimH, including the donor strand FimG peptide connected through a linker (FimH-DSG_WT).
SEQ ID NO: 60 sets forth an amino acid sequence for the mutant E. coli FimH-DSG_V27A.
SEQ ID NO: 61 sets forth an amino acid sequence for the mutant E. coli FimH-DSG_G15A_V27A.
SEQ ID NO: 62 sets forth an amino acid sequence for the mutant E. coli FimH DSG_G15A_G16A_V27A.
SEQ ID NO: 63 sets forth an amino acid sequence for the mutant E. coli FimH DSG_V27A_Q133K.
SEQ ID NO: 64 sets forth an amino acid sequence for the mutant E. coli FimH DSG_G15A_G16A_V27A_Q133K.
SEQ ID NO: 65 sets forth an amino acid sequence for the mouse Ig Kappa signal peptide sequence.
SEQ ID NO: 66 sets forth the nucleic acid sequence for chimera FimH_{LD}TMCt60HSVgD.
SEQ ID NO: 67 sets forth the amino acid sequence for chimera FimH_{LD}TMCt60HSVgD.
SEQ ID NO: 68 sets forth the nucleic acid sequence for chimera FimH_{LD}TMCtΔ5Spike.
SEQ ID NO: 69 sets forth the amino acid sequence for chimera FimH_{LD}TMCtΔ5Spike.
SEQ ID NO: 70 sets forth the nucleic acid sequence for chimera FimH_{LD}TMCtDAFgpi.
SEQ ID NO: 71 sets forth the amino acid sequence for chimera FimH_{LD}TMCtDAFgpi.
SEQ ID NO: 72 sets forth the nucleic acid sequence for secreted FimH-DSG-TM (N-deglycosylated).
SEQ ID NO: 73 sets forth the amino acid sequence for secreted FimH-DSG-TM (N-deglycosylated).
SEQ ID NO: 74 sets forth the amino acid sequence of a seven amino acid linker.
SEQ ID NO: 75 sets forth the nucleic acid sequence of a 5′ UTR.
SEQ ID NO: 76 sets forth the nucleic acid sequence of a 3′ UTR.
SEQ ID NO: 77 sets forth the modified nucleic acid sequence of the 5′ UTR set forth in SEQ ID NO: 75.
SEQ ID NO: 78 sets forth the modified nucleic acid sequence of the 5′ UTR set forth in SEQ ID NO: 76.
SEQ ID NO: 79 sets forth the amino acid sequence of SARS-CoV2 Spike protein [UniprotKB: PODTC2].
SEQ ID NO: 80 sets forth the amino acid sequence of C-terminal amino acids constituting an ER retention motif of SARS-CoV2 Spike protein [UniprotKB: PODTC2].
SEQ ID NO: 81 sets forth the amino acid sequence of Conserved charged region of C-terminal cytoplasmic tail of SARS-CoV2 Spike protein [UniprotKB: PODTC2].
SEQ ID NO: 82 sets forth the nucleic acid sequence for chimera FimHLDTMCt60HSVgD.
SEQ ID NO: 83 sets forth the nucleic acid sequence for chimera FimHLDTMCtΔ5Spike.
SEQ ID NO: 84 sets forth the nucleic acid sequence for chimera FimHLDTMCtDAFgpi.
SEQ ID NO: 85 sets forth the nucleic acid sequence for secreted FimH-DSG-TM (N-deglycosylated).
SEQ ID NO: 86 sets forth the nucleic acid sequence for a 80A polyA tail.

### DETAILED DESCRIPTION OF THE INVENTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. A ribonucleic acid polynucleotide (RNA) molecule comprising at least one open reading frame (ORF) encoding a FimH antigenic polypeptide.
E2. The RNA e of clause E1, wherein the FimH antigenic polypeptide is a full-length, truncated, fragment or variant thereof.
E3. The RNA molecule of any one of clauses E1 to E2, wherein the FimH antigenic polypeptide comprises at least one mutation.
E4. The RNA molecule of any one of clauses E1 to E3, wherein the FimH antigenic polypeptide comprises an amino acid of Table 13, including but not limited to any of SEQ ID NO: 1 to 64.
E5. The RNA molecule of any one of clauses E1-E4, wherein the FimH antigenic polypeptide comprises FimH-DSG (SEQ ID NO: 59), FimH-DSG triple mutant (G15A, G16A, V27A) (SEQ ID NO: 62), FimHLD triple mutant (G15A, G16A, V27A) (SEQ ID NO: 54), or an immunogenic fragment thereof.
E6. The RNA molecule of any one of clauses E1 to E5, wherein the FimH polypeptide has at least 90%, 95, 96%, 97%, 98% or 99% identity to the amino acid sequence selected from SEQ ID NO: 1 to 64.
E7. The RNA molecule of any one of clauses E1-E6, wherein the RNA is fused to a C-terminal membrane targeting domain.
E8. The RNA molecule of clause E7, wherein the RNA molecule and the C-terminal membrane targeting domain are separated by a linker.
E9. The RNA molecule of clause E8, wherein the linker has the amino acid sequence GGSSGGG (SEQ ID NO: 74).
E10. The RNA molecule of any of clauses E7-E9, wherein the C-terminal membrane targeting domain is derived from a viral glycoprotein.
E11. The RNA molecule of clause E10, wherein the viral glycoprotein is selected from the group consisting of HSV gD, SARS-CoV2 Spike protein, and human DAFgpi.
E12. The RNA of clause E9, wherein the C-terminal membrane targeting domain is an E. coli G-peptide.
E13. The RNA molecule of any of clauses E1-E12, wherein the open reading frame is codon-optimized.
E14. The RNA molecule of clause E11, wherein the FimH antigenic polypeptide comprises an amino acid of Table 1, including but not limited to any of SEQ ID NO: 82, SEQ ID NO: 83, and SEQ ID NO: 84.
E15. The RNA molecule of clause E14, wherein the FimH antigenic polypeptide comprises an amino acid having SEQ ID NO: 84.
E16. The RNA molecule of clause E12, wherein the FimH antigenic polypeptide comprises an amino acid having SEQ ID NO: 85.
E17. The RNA molecule of any one of clauses E1 to E16, wherein the open reading frame is transcribed from a nucleic acid sequence of Table 2, including but not limited to any of SEQ ID NO: 66, SEQ ID NO: 68, SEQ ID NO: 70 or SEQ ID NO: 72.
E18. The RNA molecule of any one of clauses E1 to E17, wherein the open reading frame comprises a nucleic acid sequence of Table 3, including but not limited to any of SEQ ID NO: 82 to 85.
E19. The RNA of clause E18, wherein each uridine of any of SEQ ID NO: 82 to 85 is replaced by 1-methyl-3′-pseudouridylyl (4P).
E20. The RNA molecule of any one of clauses E1 to E19, further comprising a 5′ untranslated region (5′ UTR).
E21. The RNA molecule of clause E21, wherein the 5′ UTR comprises nucleotides having SEQ ID NO: 75.
E22. The RNA molecule any one of clauses E1 to E21, further comprising a 3′ untranslated region (3′ UTR).
E23. The RNA molecule of clause E22, wherein the 3′ UTR comprises nucleotides having SEQ ID NO: 76.
E24. The RNA molecule of any one of clauses E1 to E23, wherein the RNA molecule comprises a 5′ cap moiety.
E25. The RNA molecule of clause E24, wherein the 5′ cap moiety is m7G(5′)ppp(5′)(2′OMeA)pG.
E26. The RNA molecule of any one of clauses E1 to E25, further comprising a 3′ poly-A tail.
E27. The RNA of clause E26, wherein the poly A tail comprises a sequence having SEQ ID NO: 86.
E28. The RNA molecule of any one of clauses E1 to E27, wherein the RNA molecule comprises a 5′ UTR and 3′ UTR.
E29. The RNA molecule of any one of clauses E1 to E28, wherein the RNA molecule comprises a 5′ cap, 5′ UTR, and 3′ UTR.
E30. The RNA molecule of any one of clauses E1 to E29, wherein the RNA molecule comprises a 5′ cap, 5′ UTR, 3′ UTR, and poly-A tail.
E31. The RNA molecule of any of clauses E1 to E30, wherein the RNA molecule comprises stabilized RNA.
E32. The RNA molecule of any one of clauses E1 to E31, wherein the RNA comprises at least one modified nucleotide.
E33. The RNA molecule of clause 3E2, wherein the modified nucleotide is pseudouridine, 1-methyl-3′-pseudouridylyl, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, or 5-methoxyuridine OR 2′-O-methyl uridine.
E34. The RNA molecule of clause E33, wherein the modified nucleotide is 1-methyl-3′-pseudouridylyl (4P).
E35. The RNA molecule of any one of clauses E1 to E25, wherein the RNA is mRNA.
E36. A composition comprising the RNA molecule of any one of clauses E1 to E35, wherein the RNA molecule is formulated in a lipid nanoparticle (RNA-LNP).
E37. The composition of clause E36, wherein lipid nanoparticle comprises at least one of a cationic lipid, a PEG-lipid, a neutral lipid, and a steroid or steroid analog.
E38. The composition of clause E36 or E37, wherein the lipid nanoparticle comprises a cationic lipid.
E39. The composition of clause E38, wherein the cationic lipid is (4-hydroxybutyl)azanediyl)bis(hexane-6,1-diyl)bis(2-hexyldecanoate) (ALC-0315).
E40. The composition of any one of clauses E36 to E39, wherein the lipid nanoparticle comprises a PEG-lipid.
E41. The composition of clause E40, wherein the PEG-lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG,N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate.
E42. The composition of clause E40 or E41, wherein the PEG-lipid is 2-[(polyethylene glycol)-2000]-N,N-ditetradecylacetamide (ALC-0159).
E43. The composition of any one of clauses E36 to E42, wherein the lipid nanoparticle comprises a neutral lipid.
E44. The composition of clause E43, wherein the neutral lipid is distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).
E45. The composition of clause E43 or E44, wherein the neutral lipid is 1,2-distearoyl-sn-glycero-3-phosphocholine (DSPC).
E46. The composition of any one of clauses E36 to E45, wherein the lipid nanoparticle comprises a steroid or steroid analog.
E47. The composition of clause 46, wherein the steroid or steroid analog is cholesterol.
E48. The composition of any one of clauses E36 to E47, wherein lipid nanoparticle wherein has a mean diameter of about 1 to about 500 nm.
E49. The composition of any one of clauses E36 to E48, wherein the composition is a vaccine.
E50. The composition of any one of clauses E36 to E49, wherein the lipid nanoparticle size is at least 40 nm.
E51. The composition of any one of clauses E36 to E49, wherein the lipid nanoparticle size is at most 180 nm.
E52. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of the RNA molecule, RNA-LNP and/or vaccine of any one of clauses E1 to E51.
E53. The method of clause E52, wherein the subject is at risk of developing a urinary tract infection.
E54. The method of clause E52, wherein the subject is at risk of developing bacteremia.
E55. The method of clause E52, wherein the subject is at risk of developing urosepsis.
E56. The method of clause E52, wherein the subject is at risk of developing cystitis.
E57. Use of the RNA molecule, RNA-LNP and/or composition of any one of clauses E1 to E56 in the manufacture of a medicament for use in (i) inducing an immune response in a subject against extra-intestinal pathogenic E. coli, or (ii) inducing the production of opsonophagocytic and/or neutralizing antibodies in a subject that are specific to extra-intestinal pathogenic E. coli.
E58. The use of clause E57, wherein the infection, disease or condition is a urinary tract infection.
E59. The use of clause E57, wherein the subject is at risk of developing bacteremia.
E60. The use of clause E57, wherein the subject is at risk of developing sepsis.
E61. The use of clause E57, wherein the subject is at risk of developing cystitis.
E62. The method or use of any one of clauses E52 to E61, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.
E63. The method or use of any one of clauses E52 to E61, wherein the subject is about 50 years of age or older.
E64. The method or use of any one of clauses E52 to E61, wherein the subject is a pregnant woman.
E65. The method or use of any one of clauses E52 to E64, wherein the RNA molecule or composition is administered as a vaccine.
E66. The method or use of any one of clauses E52 to E65, wherein the RNA molecule or composition is administered by intradermal or intramuscular injection.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

### I. Examples of Definitions

Unless otherwise defined herein, scientific and technical terms used in connection with the present invention have the meanings that are commonly understood by those of ordinary skill in the art.

Throughout this application, the term “about” is used according to its plain and ordinary meaning in the area of cell and molecular biology to indicate a deviation of ±10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a β-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease. In specific aspects, the pathogenic are peptide or protein antigens derived from E. coli FimH. Conditions and/or diseases that may be treated with RNA disclosed herein include, but are not limited to, those caused and/or impacted by bacterial infection. Such bacteria include, but are not limited to, E. coli. “Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues.

Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “NT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. The term “mutant” of a wild-type E. coli FimH protein, “mutant” of a E. coli FimH protein, “E. coli FimH protein mutant,” or “modified E. coli FimH protein” refers to a polypeptide that displays introduced mutations relative to a wild-type FimH protein and is immunogenic against the wild-type FimH protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a bacteria). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more therapeutic regimens (e.g., two or more therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity).

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

### II. E. coli Fimbrial Antigen H (FimH)

As used herein, the term “FimH antigenic polypeptide” includes any FimH polypeptide or immunogenic mutant thereof, including but not limited to, the FimH polypeptides set forth in SEQ ID Nos:1-64, 67, 69, 71 or 73.

As used herein, the term “E. coli polypeptide” includes any E. coli polypeptide. In a preferred embodiment, the E. coli polypeptide is a fimbrial antigen. In a preferred embodiment, the E. coli fimbrial antigen is FimH.

FimH antigenic polypeptides are described in PCT International Publication No. WO2022/137078, which is hereby incorporated by reference in its entirety.

Embodiments of the present disclosure provide RNA (e.g., mRNA) vaccines that include polynucleotide encoding an E. coli FimH antigen. E. coli FimH RNA vaccines, as provided herein may be used to induce a balanced immune response, comprising both cellular and humoral immunity.

Some embodiments provide E. coli vaccines comprising one or more RNA polynucleotides having an open reading frame encoding a FimH protein and a pharmaceutically acceptable carrier or excipient, formulated within a cationic lipid nanoparticle. In some embodiments, the FimH protein is selected from FimH-DSG, FimH-DSG triple mutant (G15A, G16A, V27A) or FimH_{LD} triple mutant (G15A, G16A, V27A).

As used herein the term “TM” when used in conjunction with an antigen shall mean a triple mutant, specifically a triple mutant of FimH_{LD} or FimH-DSG polypeptides having mutations at amino acid positions G15A, G16A, and V27A. Accordingly, the terms “FimH-DSG triple mutant (G15A, G16A, V27A)” and “FimH-DSG™” are interchangeable. In addition, the terms “FimH_{LD} triple mutant (G15A, G16A, V27A)” and “FimH_{LD} TM” are interchangeable.

As used herein, the abbreviation “Ct” shall mean the C-terminal domain of a polypeptide or polynucleotide.

Some embodiments provide methods of preventing or treating E. coli infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the RNA (e.g., mRNA) polynucleotides or portions thereof may encode one or more polypeptides or fragments thereof of E. coli FimH as an antigen.

### III. RNA Molecule

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) OFRs, which may be a sequence of codons that is translatable into a polypeptide or protein of interest.

A number of mRNA vaccine platforms are available in the prior art. The basic structure of in vitro transcribed (IVT) mRNA closely resembles “mature” eukaryotic mRNA and consists of (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail.

The non-coding structural features play important roles in the pharmacology of mRNA and can be individually optimized to modulate the mRNA stability, translation efficiency, and immunogenicity.

By incorporating modified nucleosides, mRNA transcripts referred to as “nucleoside-modified mRNA” or “modRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antibacterial pathways that are induced by type IFNs and are programmed to degrade and inhibit invading mRNA. For instance, the replacement of uridine with pseudouridine in in vitro transcribed (IVT) mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the invention relates to an immunogenic composition comprising an mRNA molecule that encodes one or more polypeptides or fragments thereof of E. coli FimH as an antigen. In some embodiments, the mRNA molecule comprises a nucleoside-modified mRNA.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence.

In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA.

In some aspects, the RNA molecule includes from about 20 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides. mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3-terminus of the first region (e.g., a 3′-UTR), at least one 5′-cap region, and a 3′-stabilizing region. In some embodiments, the mRNA of the invention further includes a poly-A region or a Kozak sequence (e.g., in the 5′-UTR). In some cases, mRNA of the invention may contain one or more intronic nucleotide sequences capable of being excised from the polynucleotide. In some embodiments, mRNA of the invention may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3-stabilizing region may contain an alternative nucleoside such as an L-nucleoside, an inverted thymidine, or a 2′-0-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

In some embodiments, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a payload (e.g., an E. coli FimH protein); a 3′ untranslated region (3′ UTR); and a Poly-A sequence.

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 6.0:1, about 6.5:1, or about 7.0:1.

### A. Modified Nucleobases

In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

mRNA of the invention may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the invention may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity.

mRNA of the invention may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof.

mRNA of the invention may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may be any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3-terminal alteration. In some embodiments, the polynucleotide includes an alteration at the 3-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thiopseudouridine (s4ψ), 2-thiopseudouridine (s2ψ), 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nmVu), 5-methylaminomethyl-uracil (mnm⁵U), 5-methylaminomethyl-2-thio-uracil (mnmVu), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnmVu), 5-propynyl-uracil, 1-propynyl-pseudouracil, 5-taurinomethyl-uracil (xm⁵U), 1-taurinomethyl-pseudouridine, 5-taurinomethyl-2-thio-uracil(xm⁵s²U), 1-taurinomethyl-4-thio-pseudouridine, 5-methyl-uracil (m⁵U, i.e., having the nucleobase deoxythymine), 1-methyl-pseudouridine (mV), 5-methyl-2-thio-uracil (m⁵s²U), 1-methyl-4-thio-pseudouridine (ms4ψ), 4-thio-1-methyl-pseudouridine, 3-methyl-pseudouridine (m ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, NI-methyl-pseudouridine, 3-(3-amino-3-carboxypropyl)uracil (acpU), I-methyl-3-(3-amino-3-carboxypropyl)pseudouridine (acp ψ), 5-(isopentenylaminomethyl)uracil (inm5U), 5-(isopentenylaminomethyl)-2-thio-uracil (inm5s2U), 5,2′-0-dimethyl-uridine (m5Um), 2-thio-2′-0_methyl-uridine (s2Um), 5-methoxycarbonylmethyl-2′-O-methyl-uridine (mem Um), 5-carbamoylmethyl-2′-0-methyl-uridine (ncm5Um), 5-carboxymethylaminomethyl-2′-0-methyl-uridine (cmnm5Um), 3,2′-0-dimethyl-uridine (mUm), and 5-(isopentenylaminomethyl)-2′-0-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil. Pseudouridine” is one example of a modified nucleoside that is an isomer of uridine, where the uracil is attached to the pentose ring via a carbon-carbon bond instead of a nitrogen-carbon glycosidic bond.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocy tidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f5Cm), N4,N4,2′-0-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2 -amino-purine, 7-deaza-8-aza-2-amino-purine, 7-deaza-2,6-diaminopurine, 7-deaza-8-aza-2,6-diaminopurine, 1-methy 1-adenine (ml A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-0-trimethyl-adenosine (m62Am), 1,2′-0-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-0-methyl-guanosine (m2Gm), N2,N2-dimethyl-2′-0-methyl-guanosine (m22Gm), 1-methyl-2′-0-methyl-guanosine (mlGm), N2,7-dimethyl-2′-0-methyl-guanosine (m2,7Gm), 2′-0-methyl-inosine (Im), 1,2′-0-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a] I,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

In some aspects, the RNA molecule comprises a nucleic acid sequence having at least one uridine replaced by pseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by pseudouridine. In some aspects, the RNA molecule comprises a nucleic acid sequence having all uridines replaced by pseudouridine.

### B. 5′ CAP

The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-0-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as a-methyl-phosphonate and seleno-phosphate nucleotides. Additional alterations include, but are not limited to, 2′-0-methylation of the ribose sugars of 5-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3-0-methyl group (i.e., N7,′-0-dimethyl-guanosine-5′-triphosphate-5′-guanosine, m7G-3′mppp-G, which may equivalently be designated 3′ 0-Me-m7G(5′)ppp(5′)G). The 3-0 atom of the other, unaltered, guanosine becomes linked to the 5′-terminal nucleotide of the capped polynucleotide (e.g., an mRNA). The N7- and 3′-0-methylated guanosine provides the terminal moiety of the capped polynucleotide (e.g., mRNA). Another exemplary cap is mCAP, which is similar to ARCA but has a 2′-0-methyl group on guanosine (i.e., N7,2′-0-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phosphoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5)ppp(5′)G and a N7-(4-chlorophenoxyethyl)-m3′-OG(5)ppp(5′)G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-0-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-0-methyl. Such a structure is termed the CapI structure. This cap results in a higher translational-competency, cellular stability, and a reduced activation of cellular pro-inflammatory cytokines, as compared, e.g., to other 5′ cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction. 5′-terminal caps may include endogenous caps or cap analogs. A 5′-terminal cap may include a guanosine analog. Useful guanosine analogs include inosine, NI-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

C. Untranslated Regions (UTRs) The 5′ UTR is a regulatory region situated at the 5′ end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

To alter one or more properties of an mRNA, 5′UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′ UTR may have on the mRNA. Variants of the 5′UTRs may be utilized wherein one or more nucleotides are added or removed to the termini, including A, T, C or G. 5′UTRs may also be codon-optimized, or altered in any manner described herein.

In some aspects, the RNA molecule includes a 5′ untranslated region (5′-UTR). In some aspects, the 5′ UTR comprises a sequence selected from any of SEQ ID NO: 75 or 77. In some aspects, the 5′ UTR comprises a sequence having at least 90%, 91%, 92%, 93%, 94%, 95, 96%, 97%, 98% or 99% or higher identity to any of SEQ ID NO: 75 or 77. In some aspects, the 5′ UTR comprises a sequence selected from any of SEQ ID NO: 75 or 77. In some aspects, the 5′ UTR comprises a sequence consisting of any of SEQ ID NO: 75 or 77.

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a XhoI site.

In some aspects, the RNA molecules and RNA-LNPs include a 3′ untranslated region (3′-UTR). In some aspects, the 3′ UTR comprises a sequence selected from any of SEQ ID NO: 76 or 78. In some aspects, the 3′ UTR comprises a sequence having at least 90%, 91%, 92%, 93%, 94%, 95, 96%, 97%, 98% or 99% or higher identity to any of SEQ ID NO: 76 or 78. In some aspects, the 3′ UTR comprises a sequence selected from any of SEQ ID NO: 76 or 78. In some aspects, the 3′ UTR comprises a sequence consisting of any of SEQ ID NO: 76 or 78. mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio(U). In yet other cases, a mRNA, which includes the histone stem loop may be stabilized by the addition of an oligonucleotide that terminates in a 3′-deoxynucleoside, 2′,3′-dideoxynucleoside 3′-0-methylnucleosides, 3-0-ethylnucleosides, 3′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

### D. Open Reading Frame (ORF)

The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, which are known in the art. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

The present disclosure provides for an RNA molecule comprising at least one open reading frame encoding an E. coli FimH polypeptide as described herein. In some aspects, an RNA molecule comprising at least one open reading frame encoding an E. coli FimH protein as described herein.

### E. Genes of Interest

The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. The sequence for a particular gene of interest is readily identified by one of skill in the art using public and private databases, e.g., GENBANK®.

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

The term “antigen” may refer to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g. an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from E. coli fimbrial antigen (FimH).

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

In some aspects, the RNA molecule encodes a FimH protein comprising the sequence of any one of SEQ ID NOs: 67, 69, 71 or 73, or a fragment or variant thereof.

In some aspects, the RNA molecule encodes an E. coli FimH protein synthesized from the nucleic acid sequence comprising any one of SEQ ID NOs: 66, 68, 70, 72 or 82-85, or fragment or variant thereof.

### F. Poly-A Tail

In some aspects, RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule.

Immediately after transcription, the 3-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 30, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3-end using alternative nucleotides at the 3-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′ -dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligio(U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

In one aspect, an RNA disclosed herein comprises a poly-A tail comprising a sequence having at least, at most, exactly, or between any two of 99%, 98%, 97%, 96%, 95%, 90%, 85%, or 80% identity to SEQ ID NO: 86. In one aspects, the poly-A tail comprises a sequence of SEQ ID NO: 86.

### IV. RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include, e.g., but are not limited to mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, RNase inhibitor, pyrophosphatase, and/or T7 RNA polymerase. In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor may contain at least, at most, exactly, or between any two of 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration at least, at most, exactly, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg2+, Na+, Ca2+), RNA fragments, residual nucleotides, free phosphates, or any combinations thereof.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for the ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to at least, at most, exactly, or between any two of 30 kDa, 40 kDa, 50 kDa, 60 kDa, 70 kDa, 80 kDa, 90 kDa, or more. In some aspects, a TFF membrane may have a MWCO equal to at least, at most, exactly, or between any two of 100 kDa, 150 kDa, 200 kDa, 250 kDa, 300 kDa, 350 kDa, 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to at least, at most, exactly, or between any two of 2, 3, 4, 5, 6, 7, 8, 9, 10, or more. In some aspects, the second diafiltration step is conducted with diavolumes equal to at least, at most, exactly, or between any two of 5, 6, 7, 8, 9, 10, 11, 12, 13, 14, 15, 16, 17, 18, 19, 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to at least, at most, exactly, or between any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size of at least, at most, exactly, or between any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

### V. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs may be designed to protect RNA molecules (e.g., mRNA) from extracellular RNases and/or may be engineered for systemic delivery of the RNA to target cells. In some aspects, such LNPs may be particularly useful to deliver RNA molecules (e.g., mRNA, modRNA) when RNA molecules are intravenously administered to a subject in need thereof. In some aspects, such LNPs may be particularly useful to deliver RNA molecules (e.g., mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., an E. coli FimH protein) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that may be used to deliver an active agent or therapeutic agent, such as a nucleic acid (e.g., mRNA, modRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA, modRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA, modRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, provided RNA molecules (e.g., mRNA, modRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” “size” or “mean size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have, have at least, or have at least, at most, exactly, or between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein may be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than it is achieved in the absence of interactions between nucleic acids and at least one of the lipid components.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T½ of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T½ of the LNP, LNP suspension, lyophilized LNP composition, or LNP formulation of the present disclosure is longer than the T½ of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T½” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid.

In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

### A. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### B. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, the LNPs encompass, or encapsulate, the nucleic acid molecules.

i. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may be at least, at most, exactly, or between any two of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3 -dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DM A), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-I-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-I-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-I-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[ bis(2 hydroxydodecyl)amino]ethyl]piperazin-1-yl]ethyl]amino]dodecan-2-ol (lipidoid 02-200); or heptadecan-9-yl 8-((2-hydroxyethyl) (6-oxo-6-(undecyloxy)hexyl) amino) octanoate (SM-102).

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

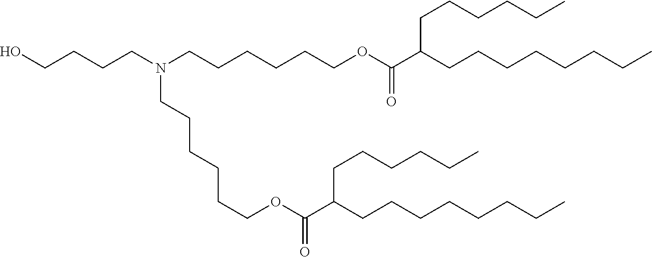 [SMILES: CCCCCCCCC(CCCCCC)C(=O)OCCCCCCN(CCCCO)CCCCCCOC(=O)C(CCCCCC)CCCCCCCC]

Cationic lipids are disclosed in, e.g., U.S. Pat. No. 10,166,298, the full disclosures of which are herein incorporated by reference in their entirety for all purposes. Representative cationic lipids include:

| No. | Structure |
|---|---|
| 1 | 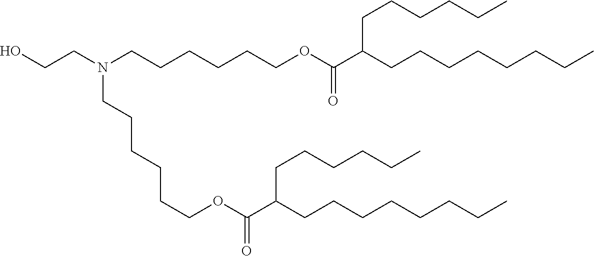 [SMILES: CCCCCCCCC(CCCCCC)C(=O)OCCCCCCN(CCO)CCCCCCOC(=O)C(CCCCCC)CCCCCCCC] |
| 2 | 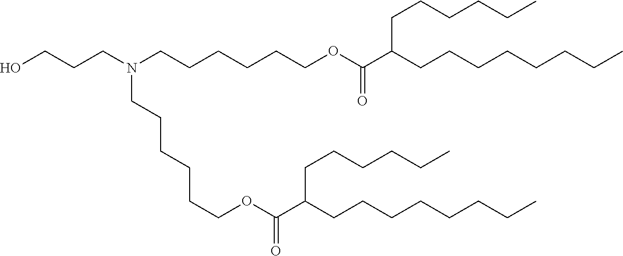 [SMILES: CCCCCCCCC(CCCCCC)C(=O)OCCCCCCN(CCCO)CCCCCCOC(=O)C(CCCCCC)CCCCCCCC] |
| 3 | 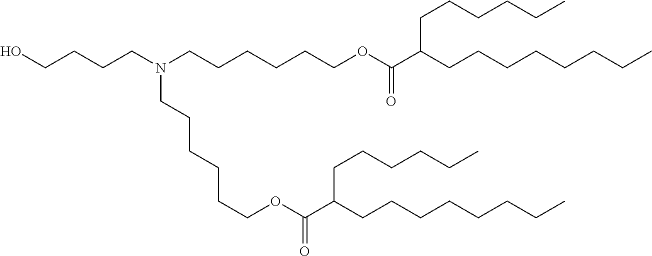 [SMILES: CCCCCCCCC(CCCCCC)C(=O)OCCCCCCN(CCCCO)CCCCCCOC(=O)C(CCCCCC)CCCCCCCC] |
| 4 | 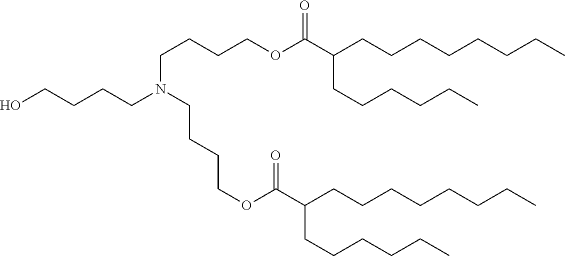 [SMILES: CCCCCCCCC(CCCCCC)C(=O)OCCCCN(CCCCO)CCCCOC(=O)C(CCCCCC)CCCCCCCC] |
| 5 | 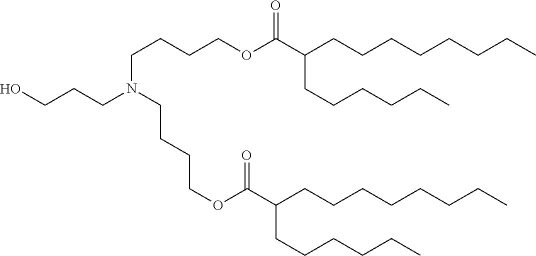 [SMILES: CCCCCCCCC(CCCCCC)C(=O)OCCCCN(CCCO)CCCCOC(=O)C(CCCCCC)CCCCCCCC] |
| 6 | 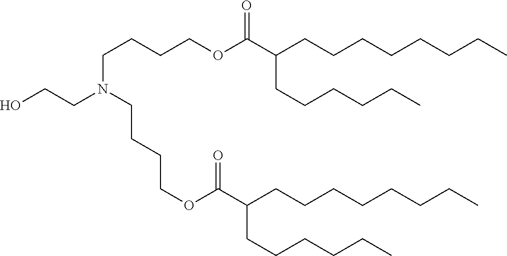 [SMILES: CCCCCCCCC(CCCCCC)C(=O)OCCCCN(CCO)CCCCOC(=O)C(CCCCCC)CCCCCCCC] |
| 7 | 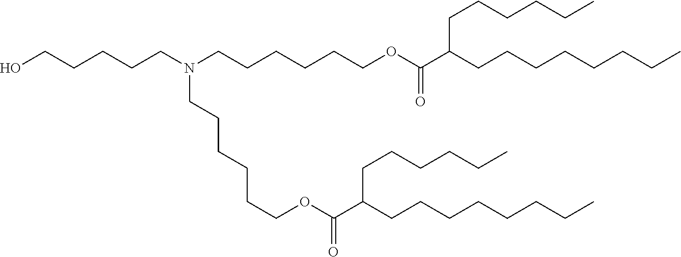 [SMILES: CCCCCCCCC(CCCCCC)C(=O)OCCCCCCN(CCCCCO)CCCCCCOC(=O)C(CCCCCC)CCCCCCCC] |
| 8 | 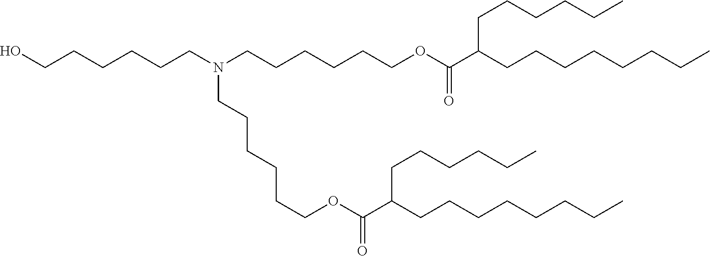 [SMILES: CCCCCCCCC(CCCCCC)C(=O)OCCCCCCN(CCCCCCO)CCCCCCOC(=O)C(CCCCCC)CCCCCCCC] |
| 9 | 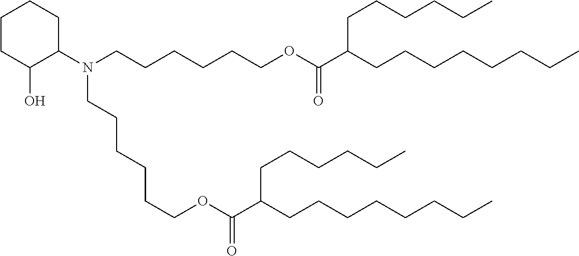 [SMILES: CCCCCCCCC(CCCCCC)C(=O)OCCCCCCN(CCCCCCOC(=O)C(CCCCCC)CCCCCCCC)C1CCCCC1O] |
| 10 | 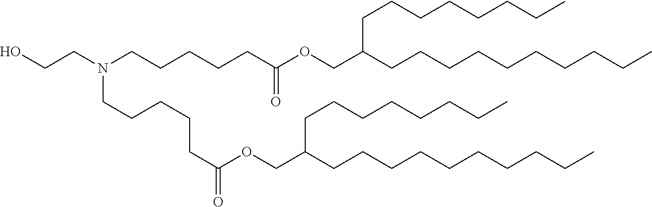 [SMILES: CCCCCCCCCCC(CCCCCCCC)COC(=O)CCCCCN(CCO)CCCCCC(=O)OCC(CCCCCCCC)CCCCCCCCCC] |
| 11 | 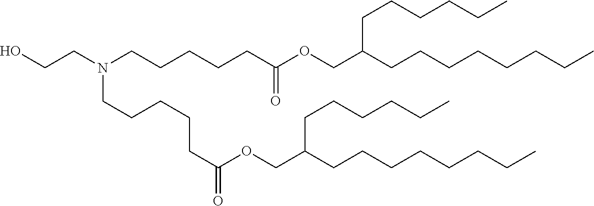 [SMILES: CCCCCCCCC(CCCCCC)COC(=O)CCCCCN(CCO)CCCCCC(=O)OCC(CCCCCC)CCCCCCCC] |
| 12 | 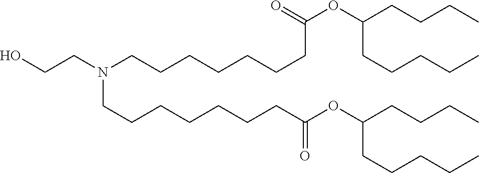 [SMILES: CCCCCC(CCCC)OC(=O)CCCCCCCN(CCO)CCCCCCCC(=O)OC(CCCC)CCCCC] |
| 13 | 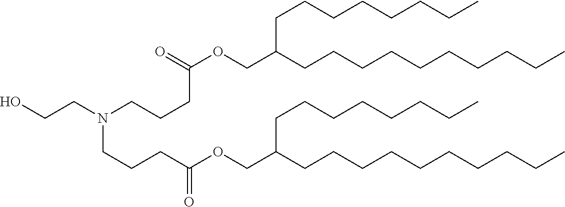 [SMILES: CCCCCCCCCCC(CCCCCCCC)COC(=O)CCCN(CCO)CCCC(=O)OCC(CCCCCCCC)CCCCCCCCCC] |
| 14 | 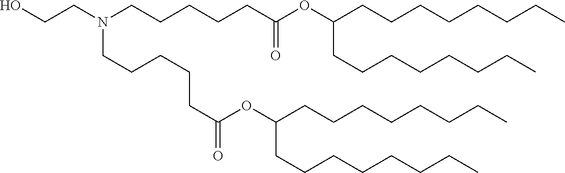 [SMILES: CCCCCCCCC(CCCCCCCC)OC(=O)CCCCCN(CCO)CCCCCC(=O)OC(CCCCCCCC)CCCCCCCC] |
| 15 | 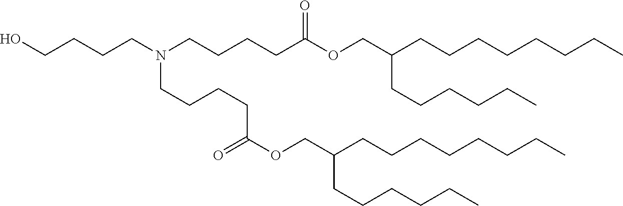 [SMILES: CCCCCCCCC(CCCCCC)COC(=O)CCCCN(CCCCO)CCCCC(=O)OCC(CCCCCC)CCCCCCCC] |
| 16 | 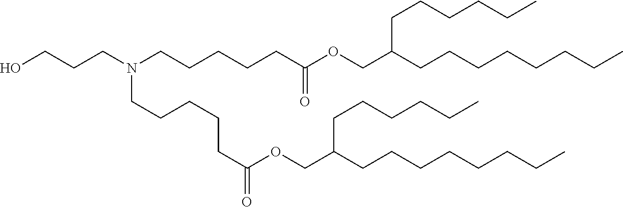 [SMILES: CCCCCCCCC(CCCCCC)COC(=O)CCCCCN(CCCO)CCCCCC(=O)OCC(CCCCCC)CCCCCCCC] |
| 17 | 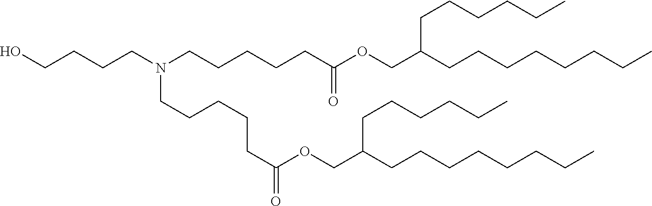 [SMILES: CCCCCCCCC(CCCCCC)COC(=O)CCCCCN(CCCCO)CCCCCC(=O)OCC(CCCCCC)CCCCCCCC] |
| 18 | 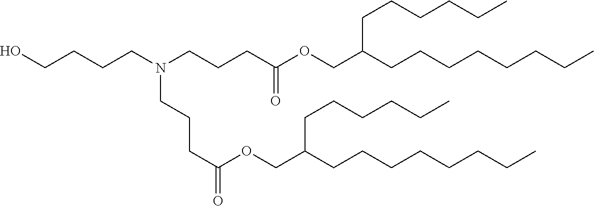 [SMILES: CCCCCCCCC(CCCCCC)COC(=O)CCCN(CCCCO)CCCC(=O)OCC(CCCCCC)CCCCCCCC] |
| 19 | 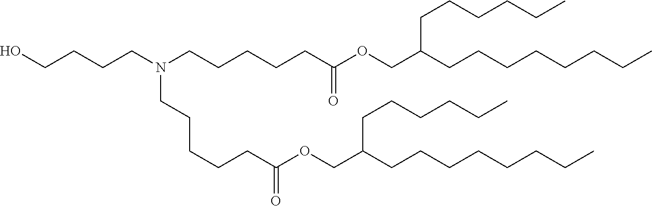 [SMILES: CCCCCCCCC(CCCCCC)COC(=O)CCCCCN(CCCCO)CCCCCC(=O)OCC(CCCCCC)CCCCCCCC] |
| 20 | 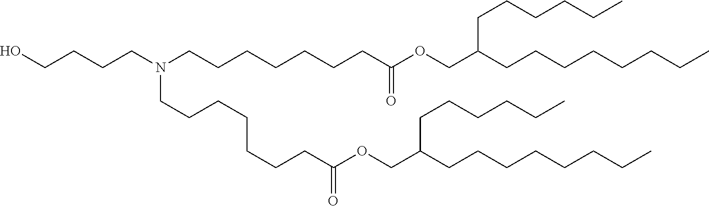 [SMILES: CCCCCCCCC(CCCCCC)COC(=O)CCCCCCCN(CCCCO)CCCCCCCC(=O)OCC(CCCCCC)CCCCCCCC] |
| 21 | 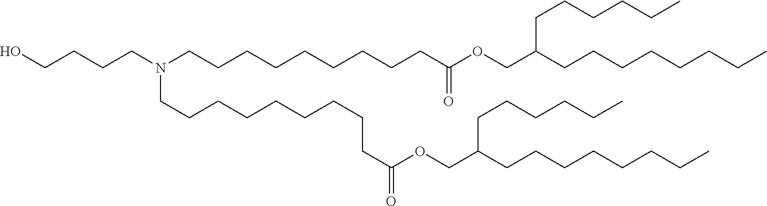 [SMILES: CCCCCCCCC(CCCCCC)COC(=O)CCCCCCCCCN(CCCCO)CCCCCCCCCC(=O)OCC(CCCCCC)CCCCCCCC] |
| 22 | 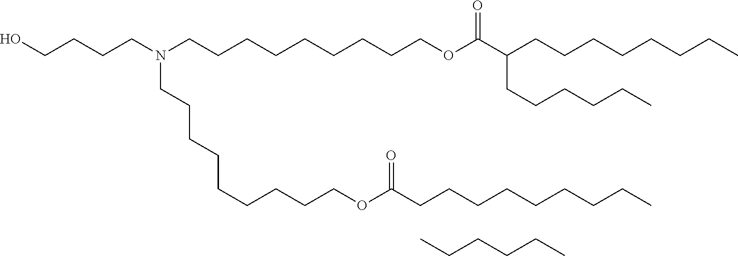 |
| 23 | 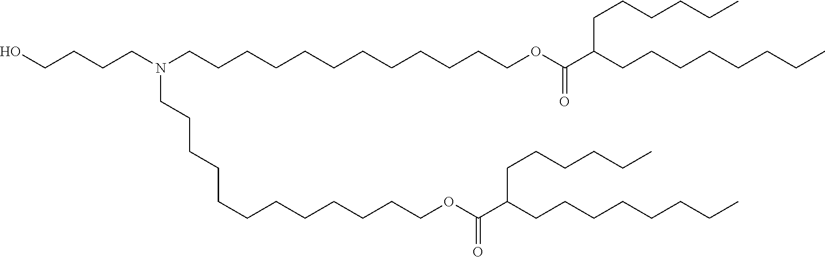 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 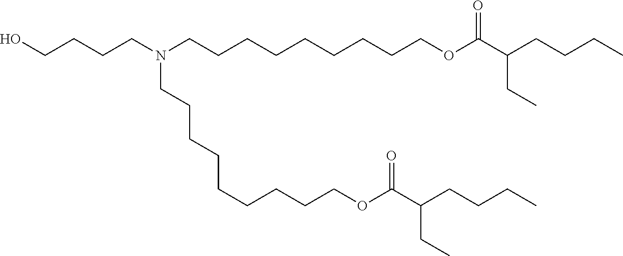 [SMILES: CCCCC(CC)C(=O)OCCCCCCCCCN(CCCCO)CCCCCCCCCOC(=O)C(CC)CCCC] |
| 25 | 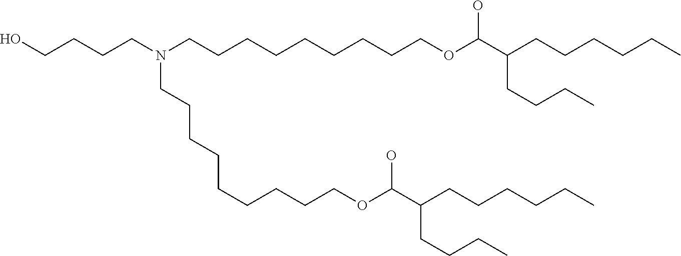 [SMILES: CCCCCCC(CCCC)C(O)OCCCCCCCCCN(CCCCO)CCCCCCCCCOC(O)C(CCCC)CCCCCC] |
| 26 | 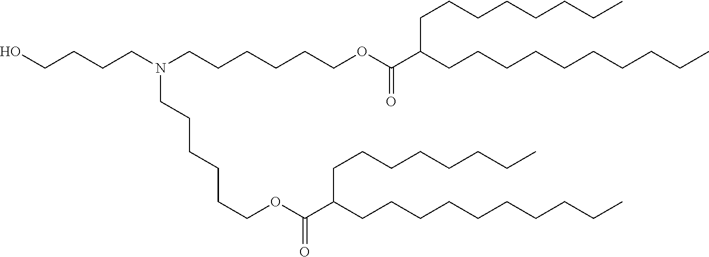 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 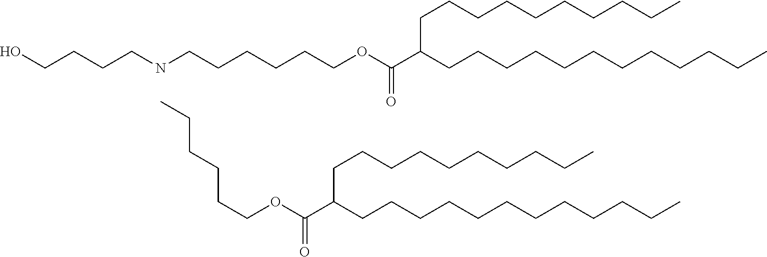 |
| 28 | 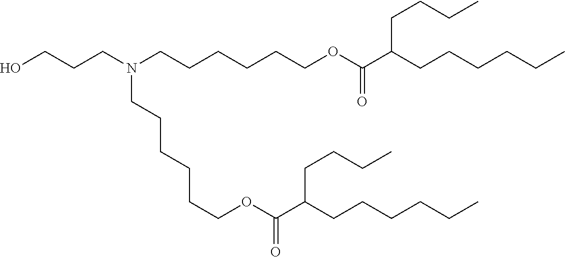 [SMILES: CCCCCCC(CCCC)C(=O)OCCCCCCN(CCCO)CCCCCCOC(=O)C(CCCC)CCCCCC] |
| 29 | 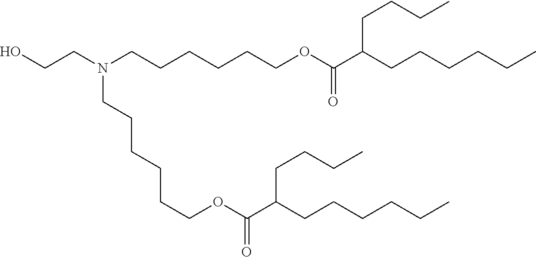 [SMILES: CCCCCCC(CCCC)C(=O)OCCCCCCN(CCO)CCCCCCOC(=O)C(CCCC)CCCCCC] |
| 30 | 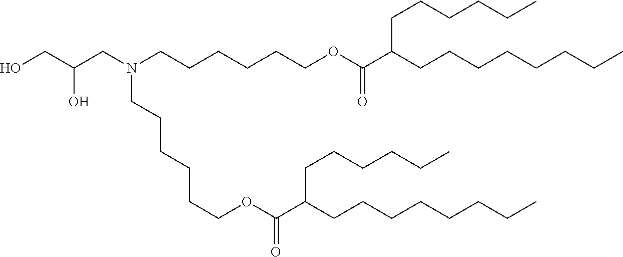 [SMILES: CCCCCCCCC(CCCCCC)C(=O)OCCCCCCN(CCCCCCOC(=O)C(CCCCCC)CCCCCCCC)CC(O)CO] |
| 31 | 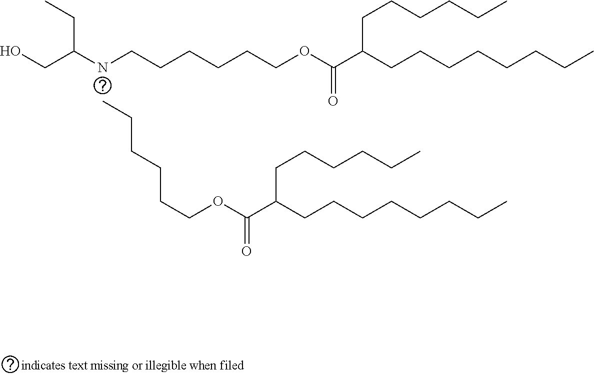 |
| 32 | 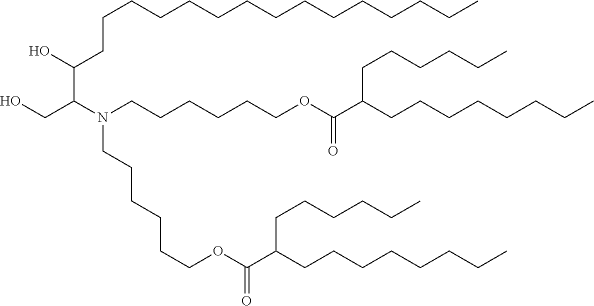 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| 33 | 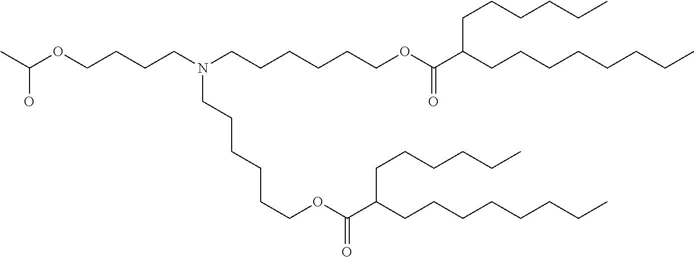 [SMILES: CCCCCCCCC(CCCCCC)C(=O)OCCCCCCN(CCCCCCOC(=O)C(CCCCCC)CCCCCCCC)CCCCOC(C)O] |
| 34 | 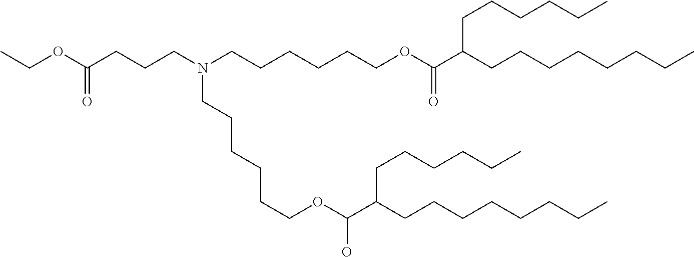 [SMILES: CCCCCCCCC(CCCCCC)C(=O)OCCCCCCN(CCCCCCOC(O)C(CCCCCC)CCCCCCCC)CCCC(=O)OCC] |
| 35 | 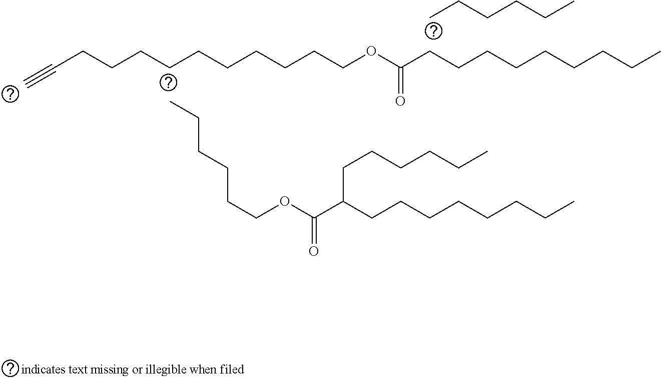 |
| 36 | 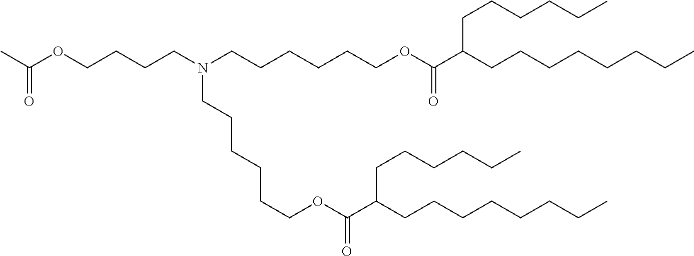 [SMILES: CCCCCCCCC(CCCCCC)C(=O)OCCCCCCN(CCCCCCOC(=O)C(CCCCCC)CCCCCCCC)CCCCOC(C)=O] |

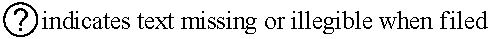

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between any two of about 40, 41, 42, 43, 44, 45, 46, 47, 48, 49, 50, 51, 52, 53, 54, 55, 56, 57, 58, 59 or 60 mole percent, respectively.

In some aspects of the disclosure the LNP comprises a combination or mixture of any the lipids described above.

ii. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]-N,N-ditetradecylacetamide, and the like.

In certain aspects, the LNP comprises an additional, stabilizing-lipid which is a polyethylene glycol-lipid (pegylated lipid). A polymer conjugated lipid (e.g. PEG-lipid) refers to a molecule comprising both a lipid portion and a polymer portion. An example of a polymer conjugated lipid is a PEG-lipid. A PEG-lipid refers to a molecule comprising both a lipid portion and a polyethylene glycol portion. PEG-lipids include, but are not limited to, PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one aspect, the polyethylene glycol-lipid is N-[(methoxy polyethylene glycol)2000)carbamyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. PEG-lipids are disclosed in, e.g., U.S. Pat. No. 9,737,619, the full disclosures of which are herein incorporated by reference in their entirety for all purposes.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

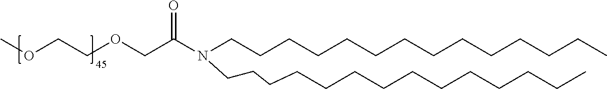

In another aspect, a polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

In various aspects, the molar ratio of the cationic lipid to the pegylated lipid or polymer lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid or polymer lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle.

iii. Additional Lipids

In certain aspects, the LNP comprises one or more additional lipids or lipid-like materials that stabilize the formation of particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DpyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC), having the formula:

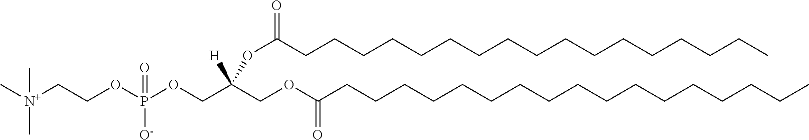 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

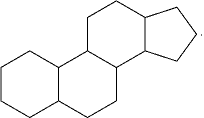 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In one aspect, the cholesterol has the formula:

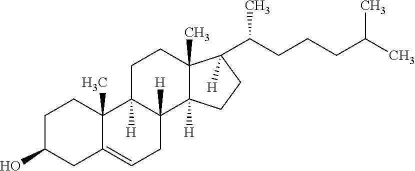

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from about 2:1 to about 8:1, or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle. In some aspects, the non-cationic lipid, e.g., neutral lipid (e.g., one or more phospholipids and/or cholesterol), may be at least, at most, exactly, or between any two of 0 mol %, 10 mol %, 20 mol %, 30 mol %, 40 mol %, 50 mol %, 60 mol %, 70 mol %, 80 mol %, or 90 mol % of the total lipid present in the particle.

### C. Other Materials

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN® 20], polyoxy ethylene sorbitan [TWEEN® 60], polyoxy ethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid.

Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond).

Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidyl glycerols, and phosphatidic acids. Phospholipids also include phosphosphingolipid, such as sphingomyelin. In some embodiments, a phospholipid useful or potentially useful in the present invention is an analog or variant of DSPC.

### VI. Characterization and Analysis of RNA Molecule

The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a m7(3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest (e.g., an antigen) is at least 2-fold to at least 10-fold. In some aspects, elevated expression of a gene of interest (e.g., an antigen) is at least 2-fold. In some aspects, elevated expression of a gene of interest (e.g., an antigen) is at least 3-fold. In some aspects, elevated expression of a gene of interest (e.g., an antigen) is at least 4-fold. In some aspects, elevated expression of a gene of interest (e.g., an antigen) is at least 6-fold. In some aspects, elevated expression of a gene of interest (e.g., an antigen) is at least 8-fold. In some aspects, elevated expression of a gene of interest (e.g., an antigen) is at least 10-fold.

In some aspects, elevated expression of a gene of interest (e.g., an antigen) is about 2-fold to about 50-fold. In some aspects, elevated expression of a gene of interest (e.g., an antigen) is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest (e.g., an antigen) is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest (e.g., an antigen) persists for at least, at most, exactly, or between any two of 24 hours, 36 hours, 48 hours, 60 hours, 72 hours, 84 hours, 96 hours, 108 hours, or 120 hours, or any range or value derivable therein.

### VII. Immune Response

As discussed herein, the disclosure concerns evoking or inducing an immune response in a subject against a FimH protein, e.g., a wild type or variant FimH protein. In one aspect, the immune response may protect against or treat a subject having, suspected of having, or at risk of developing an infection or related disease, particularly those related to E. coli FimH. One use of the immunogenic compositions of the disclosure is to prevent E. coli infections by inoculating or vaccination of a subject.

In some aspects of the disclosure, RNA molecules encoding E. coli FimH protein, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against an antigen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an of an antigen, e.g. an E. coli FimH protein encoded by an RNA molecule of the present disclosure.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as bacteria, including but not limited to E. coli.

Passive immunity may be imparted to a patient or subject by administering to the patient immunoglobulins (Ig) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against E. coli or other organism. A subject thus treated would donate plasma from which hyperimmune globulin would then be obtained, via conventional plasma-fractionation methodology, and administered to another subject in order to impart resistance against or to treat E. coli infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In some aspects, an RNA molecules and/or RNA-LNPs disclosed herein may be administered in a pharmaceutical composition or a medicament and may be administered in the form of any suitable pharmaceutical composition. In some aspects, a pharmaceutical composition is for therapeutic or prophylactic treatments. In one aspect, the disclosure relates to a composition for administration to a host. In some aspects, the host is a human. In other aspects, the host is a non-human.

In some aspects, an RNA molecules and/or RNA-LNPs disclosed herein may be administered in a pharmaceutical composition which may be formulated into preparations in solid, semi-solid, liquid, lyophilized, frozen, or gaseous forms. In some aspects, an RNA molecule and/or RNA-LNPs disclosed herein may be administered in a pharmaceutical composition which may comprise a pharmaceutically acceptable carrier and may optionally comprise one or more adjuvants, stabilizers, salts, buffers, preservatives, and optionally other therapeutic agents. In some aspects, a pharmaceutical composition disclosed herein comprises one or more pharmaceutically acceptable carriers, diluents and/or excipients. In some aspects, pharmaceutical compositions do not include an adjuvant (e.g., they are adjuvant free).

Suitable preservatives for use in a pharmaceutical compositions of the present disclosure include, without limitation, benzalkonium chloride, chlorobutanol, paraben and thimerosal. The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some aspects, the pharmaceutical composition of the present disclosure includes sodium chloride.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents may be selected with regard to the intended route of administration and standard pharmaceutical practice.

In some aspects, the composition comprises an RNA molecule comprising an open reading frame encoding an immunogenic polypeptide. In some aspects, the immunogenic polypeptide comprises an E. coli antigen. In some aspects, the E. coli antigen is an E. coli FimH protein.

In some aspects, the composition comprises an RNA molecule comprising an open reading frame encoding a full-length E. coli FimH protein. In some aspects, the encoded immunogenic polypeptide is a truncated E. coli FimH protein. In some aspects, the encoded immunogenic polypeptide is a variant of an E. coli FimH protein. In some aspects, the encoded immunogenic polypeptide is a fragment of an E. coli FimH protein.

### A. Immunogenic Compositions Including LNPS

In some aspects, a pharmaceutical composition comprises an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding a FimH polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., FimH RNA-LNPs).

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further include a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 5% to about 10% w/v. For example, the total concentration of the stabilizing agent may be equal to at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is equal to at least, at most, exactly, or between any two of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent: 1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. In one aspect, the buffer is Tris buffer. In another aspect, the buffer is a HEPES buffer. In a further aspect, the buffer is a PBS buffer. For example, the buffer concentration may be equal to at least, at most, exactly, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may be at least, at most, exactly, or between any two of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. The concentration of the salts in the composition may be about 70 mM to about 140 mM. For example, the salt concentration may be equal to at least, at most, exactly, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is equal to at least, at most, exactly, or between any two of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may be at a pH equal to at least, at most, exactly, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, or cyclodextrins.

Examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from about 10% to about 90% by weight of the total composition, about 25% to about 75%, about 30% to about 60% by weight, or about 12% to about 60%.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, a pharmaceutical composition comprises an FimH RNA molecule encoding a FimH polypeptide as disclosed herein that is complexed with, encapsulated in, and/or formulated with one or more lipids to form FimH RNA-LNPs. In some aspects, the FimH RNA-LNP composition is a liquid. In some aspects, the FimH RNA-LNP composition is frozen. In some aspects, the FimH RNA-LNP composition is lyophilized. In some aspects, a FimH RNA-LNP composition comprises a FimH RNA polynucleotide molecule encoding a FimH polypeptide as disclosed herein, encapsulated in LNPs with a lipid composition of a cationic lipid, a PEGylated lipid (i.e. PEG-lipid), and one or more structural lipids (e.g., a neutral lipid).

In some aspects, a FimH RNA-LNP composition comprises an cationic lipid. The cationic lipid may comprise any one or more cationic lipids disclosed herein. In specific aspects, the cationic lipid comprises ((4-hydroxybutyl)azanediyl)bis(hexane-6,1-diyl)bis(2-hexyldecanoate) (ALC-0315). In some aspects, the cationic lipid (e.g., ALC-0315) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the cationic lipid (e.g., ALC-0315) is included in the composition at a concentration of at least, at most, between any two of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the cationic lipid (e.g., ALC-0315) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315) is included in the composition at a concentration of 0.8 to 0.95 mg/mL. In specific aspects, the cationic lipid (e.g., ALC-0315) is included in the composition at a concentration of 0.8 to 0.9 mg/mL. In specific aspects, the cationic lipid (e.g., ALC-0315) is included in the composition at a concentration of 0.85 to 0.9 mg/mL. In specific aspects, the cationic lipid (e.g., ALC-0315) is included in the composition at a concentration of 0.8, 0.81, 0.82, 0.83, 0.84, 0.85, 0.86, 0.87, 0.88, 0.89, 0.9, 0.91, 0.92, 0.93, 0.94, or 0.95 mg/mL. Concentrations for lyophilized compositions are determined post-reconstitution.

In some aspects, a FimH RNA-LNP composition further comprises a PEGylated lipid (i.e., PEG-lipid). The PEGylated lipid may comprise any one or more PEGylated lipids disclosed herein. In specific aspects, the PEGylated lipid comprises 2-[(polyethylene glycol)-2000]-N,N-ditetradecylacetamide (ALC-0159). In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, or between 0.2 and 0.25 mg/mL.

In specific aspects, the PEGylated lipid (e.g., ALC-0159) is included in the composition at a concentration of 0.05 to 0.15 mg/mL. In specific aspects, the PEGylated lipid (e.g., ALC-0159) is included in the composition at a concentration of 0.10 to 0.15 mg/mL. In specific aspects, the PEGylated lipid (e.g., ALC-0159) is included in the composition at a concentration of 0.05, 0.06, 0.07, 0.08, 0.09, 0.1, 0.11, 0.12, 0.13, 0.14 or 0.15 mg/mL. Concentrations for lyophilized compositions are determined post-reconstitution.

In some aspects, a FimH RNA-LNP composition further comprises one or more structural lipids. The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-Distearoyl-sn-glycero-3-phosphocholine (DSPC) and cholesterol. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the one or more structural lipids (e.g., DSPC and cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC and cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the one or more structural lipids include DSPC, and the DSPC is included in the composition at a concentration of 0.1 to 0.25 mg/mL. In specific aspects, the one or more structural lipids include DSPC, and the DSPC is included in the composition at a concentration of 0.15 to 0.25 mg/mL. In specific aspects, the one or more structural lipids include DSPC, and the DSPC is included in the composition at a concentration of 0.1, 0.11, 0.12, 0.13, 0.14, 0.15, 0.16, 0.17, 0.18, 0.19, 0.2, 0.21, 0.22, 0.23, 0.24 or 0.25 mg/mL.

In specific aspects, the one or more structural lipids include cholesterol, and the cholesterol is included in the composition at a concentration of 0.3 to 0.45 mg/mL. In specific aspects, the one or more structural lipids include cholesterol, and the cholesterol is included in the composition at a concentration of 0.3 to 0.4. In specific aspects, the one or more structural lipids include cholesterol, and the cholesterol is included in the composition at a concentration of 0.35 to 0.45. In specific aspects, the one or more structural lipids include cholesterol, and the cholesterol is included in the composition at a concentration of 0.30, 0.31, 0.32, 0.33, 0.34, 0.35, 0.36, 0.37, 0.38, 0.39, 0.40, 0.41, 0.42, 0.43, 0.44, or 0.45 mg/mL. Concentrations for lyophilized compositions are determined post-reconstitution.

In some aspects, the FimH RNA-LNP composition further comprises one or more buffers and stabilizing agents, and optionally, salts. Thus, in some aspects, the FimH RNA-LNP composition comprises an cationic lipid, a PEGylated lipid, one or more structural lipids, one or more buffers, a stabilizing agent, and optionally, a salt.

In some aspects, a FimH RNA-LNP composition comprises one or more buffers. The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. Concentrations for lyophilized compositions are determined post-reconstitution.

In some aspects, the FimH RNA-LNP composition is a liquid composition comprising a Tris buffer. In some aspects, the Tris buffer comprises a first buffer. In some aspects, the first buffer is tromethamine. In some aspects, the first buffer (e.g., tromethamine) is included in the liquid composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.1, at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the first buffer (e.g., tromethamine) is included in the liquid composition at a concentration of 0.1 to 0.3 mg/mL. In specific aspects, the first buffer (e.g., tromethamine) is included in the liquid composition at a concentration of 0.15 to 0.25 mg/mL. In specific aspects, the first buffer (e.g., tromethamine) is included in the liquid composition at a concentration of 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.3 mg/mL.

In some aspects, the FimH RNA-LNP composition is a liquid composition comprising a Tris buffer comprising a second buffer. In some aspects, the second buffer comprises Tris HCl. In some aspects, the second buffer (e.g., Tris HCl) is included in the liquid composition at a concentration of at least, at most, between any two of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of 1.25 to 1.40 mg/mL. In specific aspects, the second buffer (e.g., Tris HCl) is included in the liquid composition at a concentration of 1.30 to 1.40 mg/mL. In specific aspects, the second buffer (e.g., Tris HCl) is included in the liquid composition at a concentration of 1.25, 1.26, 1.27, 1.28, 1.29, 1.30, 1.31, 1.32, 1.33, 1.34, or 1.35, 1.36, 1.37, 1.38, 1.39, or 1.40 mg/mL.

In some aspects, the FimH RNA-LNP composition is a lyophilized composition comprising a Tris buffer. In some aspects, the Tris buffer comprises a first buffer. In some aspects, the first buffer is tromethamine. In some aspects, the first buffer (e.g., tromethamine) is included in the lyophilized composition at a concentration, after reconstitution, of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of 0.01 and 0.15 mg/mL. In specific aspects, the first buffer (e.g., tromethamine) is included in the lyophilized composition at a concentration, after reconstitution, of 0.01 and 0.10 mg/mL. In specific aspects, the first buffer (e.g., tromethamine) is included in the lyophilized composition at a concentration, after reconstitution, of 0.05 and 0.15 mg/mL. In specific aspects, the first buffer (e.g., tromethamine) is included in the lyophilized composition at a concentration, after reconstitution, of 0.01, 0.02, 0.03, 0.04, 0.05, 0.06, 0.07, 0.08, 0.09, 0.1, 0.11, 0.12, 0.13, 0.14, or 0.15 mg/mL.

In some aspects, the FimH RNA-LNP composition is a lyophilized composition comprising a Tris buffer comprising a second buffer. In some aspects, the second buffer comprises Tris HCl. In some aspects, the second buffer (e.g., Tris HCl) is included in the lyophilized composition at a concentration, after reconstitution, of at least, at most, between any two of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of 0.5 and 0.65 mg/mL. In specific aspects, the second buffer (e.g., Tris HCl) is included in the lyophilized composition at a concentration, after reconstitution, of 0.5 and 0.6 mg/mL. In specific aspects, the second buffer (e.g., Tris HCl) is included in the lyophilized composition at a concentration, after reconstitution, of 0.55 and 0.65 mg/mL. In specific aspects, the second buffer (e.g., Tris HCl) is included in the lyophilized composition at a concentration, after reconstitution, of 0.5, 0.51, 0.52, 0.53, 0.54, 0.55, 0.56, 0.57, 0.58, 0.59, 0.6, 0.61, 0.62, 0.63, 0.64, or 0.65 mg/mL.

In some aspects, a FimH RNA-LNP composition comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is included in the composition at a concentration of at least, at most, between any two of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/μg/mg per mL.

In some aspects, the FimH RNA-LNP composition is a liquid composition, and the stabilizing agent (e.g., sucrose) is included in the liquid composition at a concentration of at least, at most, between any two of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125 or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of 95 to 110 mg/mL. In specific aspects, the stabilizing agent (e.g., sucrose) is included in the liquid composition at a concentration of 95 to 105 mg/mL. In specific aspects, the stabilizing agent (e.g., sucrose) is included in the liquid composition at a concentration of 100 to 110 mg/mL. In specific aspects, the stabilizing agent (e.g., sucrose) is included in the liquid composition at a concentration of 95, 96, 97, 98, 99, 100, 101, 102, 103, 104, 105, 106, 107, 108, 109, or 110 mg/mL.

In some aspects, the FimH RNA-LNP composition is a lyophilized composition, and the stabilizing agent (e.g., sucrose) is included in the lyophilized composition at a concentration, after reconstitution, of at least, at most, between any two of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL. In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75 or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70 or between 70 to 80 mg/mL.

In specific aspects, the stabilizing agent (e.g., sucrose) is included in the lyophilized composition at a concentration, after reconstitution, of 35 to 50 mg/mL. In specific aspects, the stabilizing agent (e.g., sucrose) is included in the lyophilized composition at a concentration, after reconstitution, of 35 to 45 mg/mL. In specific aspects, the stabilizing agent (e.g., sucrose) is included in the lyophilized composition at a concentration, after reconstitution, of 40 to 50 mg/mL. In specific aspects, the stabilizing agent (e.g., sucrose) is included in the lyophilized composition at a concentration, after reconstitution, of 35, 36, 37, 38, 39, 40, 41, 42, 43, 44, 45, 46, 47, 48, 49 or 50 mg/mL.

In some aspects, the FimH RNA-LNP composition is a lyophilized composition, and the lyophilized FimH RNA-LNP composition further comprises a salt. The salt may comprise any one or more salts disclosed herein. In specific aspects, the salt comprises sodium chloride (NaCl). In some aspects, the salt (e.g., NaCl) is included in the lyophilized composition at a concentration, after reconstitution, of at least, at most, between any two of, or exactly 0.5, 1, 1.5, 2, 2.5, 3, 3.5, 4, 4, 5, 5, 5.5, 6, 6.5, 7, 7.5, 8, 8.5, 9, 9.5, 10, 10, 5, 11, 11, 5, 12, 12, 5, 13, 13.5, 14, 14, 5, 15, 15, 5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/μg/mg per mL. In some aspects, the salt (e.g., NaCl) is included in the lyophilized composition at a concentration, after reconstitution, of in at least, at most, between any two of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some aspects, the salt (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt (e.g., NaCl) is included in the lyophilized composition at a concentration, after reconstitution, of between 5 and 15 mg/mL. In some aspects, the salt (e.g., NaCl) is included in the lyophilized composition at a concentration, after reconstitution, of between 5 and 10 mg/mL. In specific aspects, the salt (e.g., NaCl) is included in the lyophilized composition at a concentration, after reconstitution, of 5, 6, 7, 8, 9, 10, 11, 12, 13, 14, or 15 mg/mL.

In some aspects, lyophilized compositions are reconstituted in a suitable carrier or diluent. The carrier or diluent may comprise any one or more carriers or diluents disclosed herein. In specific aspects, the carrier or diluent comprises saline, e.g., physiological saline. The saline may comprise 0.9% saline for injection. In some aspects, the lyophilized compositions are reconstituted in at least, at most, between any two of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of saline.

In specific aspects, the lyophilized compositions are reconstituted in 0.6 to 0.75 mL of saline. In specific aspects, the lyophilized compositions are reconstituted in 0.65 to 0.75 mL of saline. In specific aspects, the lyophilized compositions are reconstituted in 0.6, 0.61, 0.62, 0.63, 0.64, 0.65, 0.66, 0.67, 0.68, 0.69, 0.7, 0.71, 0.72, 0.73, 0.74 or 0.75 mL of saline.

The pH of the FimH RNA-LNP composition may be at least, at most, exactly, or between any two of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the FimH RNA-LNP composition is at a pH of at least 6.5, at least 7.0, at least 7.5, at least 8.0, or at least 8.5. In specific aspects, the FimH RNA-LNP composition is at a pH between 6.0 and 7.5, between 6.5 and 7.5, between 7.0 and 8.0, between and 7.5 and 8.5. In specific aspects, the FimH RNA-LNP composition is between 7.0 and 8.0. In specific aspects, the FimH RNA-LNP composition is at pH 7.0, 7.1, 7.2, 7.3, 7.4, 7.5, 7.6, 7.7, 7.8, 7.9 or 8.0.

In specific aspects, a FimH RNA-LNP composition comprises a FimH RNA polynucleotide encoding a FimH polypeptide as disclosed herein, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL.

In specific aspects, a FimH RNA-LNP composition comprises a FimH RNA polynucleotide encoding a FimH polypeptide as disclosed herein, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL.

In specific aspects, the FimH RNA-LNP composition is a liquid FimH RNA-LNP composition, and the liquid FimH RNA-LNP composition further comprises a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, the FimH RNA-LNP composition is a liquid FimH RNA-LNP composition, and the liquid FimH RNA-LNP composition further comprises a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

Thus, in specific aspects, a liquid FimH RNA-LNP composition comprises an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.1 to 0.3 mg/mL, a second buffer at a concentration of 1.25 to 1.4 mg/mL, and a stabilizing agent at a concentration of 95 to 110 mg/mL.

Thus, in specific aspects, a liquid FimH RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the FimH RNA-LNP composition is a lyophilized FimH RNA-LNP composition, and the lyophilized FimH RNA-LNP composition further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL.

In specific aspects, the FimH RNA-LNP composition is a lyophilized FimH RNA-LNP composition, and the lyophilized FimH RNA-LNP composition further comprises a Tris buffer composition comprising tromethamine at a concentration of 0.01 and 0.15 mg/mL, Tris HCl at a concentration of 0.5 and 0.65 mg/mL, sucrose at a concentration of 35 to 50 mg/mL, and a sodium chloride (NaCl) at a concentration of 5 to 15 mg/mL.

Thus, in specific aspects, a lyophilized FimH RNA-LNP composition comprises a cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent.

Thus, in some aspects, a lyophilized FimH RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline.

Concentrations in the lyophilized FimH RNA-LNP composition are determined post-reconstitution.

The FimH RNA-LNP compositions further comprise FimH RNA described herein encapsulated in LNPs, see section D. ADMINISTRATION.

In specific aspects, a FimH RNA-LNP composition is a liquid FimH RNA-LNP composition comprising a FimH RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, a liquid FimH RNA-LNP composition comprises a FimH RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the FimH RNA-LNP composition is a lyophilized FimH RNA-LNP composition comprising a FimH RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 mL of carrier or diluent. Concentrations in the lyophilized FimH RNA-LNP composition are determined post-reconstitution.

In specific aspects, a lyophilized FimH RNA-LNP composition comprises a FimH RNA polynucleotide encoding a FimH polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline. Concentrations in the lyophilized FimH RNA-LNP composition are determined post-reconstitution.

### B. Vaccines

In some aspects, a pharmaceutical composition described herein is an immunogenic composition for inducing an immune response. For example, in some aspects, an immunogenic composition is a vaccine. In some aspects, the compositions described herein include at least one isolated nucleic acid or polypeptide molecule as described herein. In specific aspects, the immunogenic compositions comprise nucleic acids, and the immunogenic compositions are nucleic acid vaccines. In some aspects, the immunogenic compositions comprise RNA (e.g. mRNA), and vaccines are RNA vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that may be treated with the nucleic acid and/or peptide or polypeptide compositions include, but are not limited to, those caused and/or impacted by infection, cancer, rare diseases, and other diseases or conditions caused by overproduction, underproduction, or improper production of protein or nucleic acids.

In some aspects, the composition is substantially free of one or more impurities or contaminants and, for instance, includes nucleic acid or polypeptide molecules that are equal to at least, at most, exactly, or between any two of 90%, 91%, 92%, 93%, 94%, 95%, 96%, 97%, 98%, or 99% pure; at least 98% pure, or at least 99% pure.

The present disclosure includes methods for preventing, treating or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. Immunogenic compositions, proposed to be suitable for use as a vaccine, may be prepared from RNA molecules encoding polypeptide(s), such as the E. coli FimH polypeptides described herein. In certain aspects, immunogenic compositions are lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference in their entireties. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference in their entireties.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%. In some aspects, suppositories may be formed from mixtures containing the active ingredient in the range of about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient.

The polypeptide-encoding nucleic acid constructs and polypeptides may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6 or more administrations. The vaccinations may be at 1, 2, 3, 4, 5, 6, 7, 8, to 5, 6, 7, 8, 9, 10, 11, 12 twelve week intervals, including all ranges there between. In some aspects, vaccinations may be at 1, 2, 3, 4, 5, 6, 7, 8, 9, 10, 11, 12 month intervals, including all ranges there between. Periodic boosters at intervals of 1-5 years may be desirable to maintain protective levels of the antibodies.

i. Carriers

A pharmaceutically acceptable carrier may include the liquid or non-liquid basis of a composition. If a composition is provided in liquid form, the carrier may be water, such as pyrogen-free water; isotonic saline or buffered (aqueous) solutions, e.g. phosphate, citrate buffered solutions. Water or a buffer, such as an aqueous buffer, may be used, containing a sodium salt, a calcium salt, and and/or a potassium salt. The sodium, calcium and/or potassium salts may occur in the form of their halogenides, e.g. chlorides, iodides, or bromides, in the form of their hydroxides, carbonates, hydrogen carbonates, or sulfates, etc. Examples of sodium salts include, but are not limited to, NaCl, NaI, NaBr, Na₂CO₃, NaHCO₃, Na₂SO₄, Na₂HPO₄, Na₂HPO₄·₂H₂O, examples of potassium salts include, but are not limited to, KCl, KI, KBr, K₂CO₃, KHCO₃, K₂SO₄, KH₂PO₄ and examples of calcium salts include, but are not limited to, CaCl₂, CaI₂, CaBr₂, CaCO₃, CaSO₄, Ca(OH)₂. Examples of further carriers may include sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. Examples of further carriers may include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

ii. Adjuvants

Suitable adjuvants include all acceptable immunostimulatory compounds, such as cytokines, toxins, or synthetic compositions. A number of adjuvants may be used to enhance an antibody response. Adjuvants include, but are not limited to, oil-in-water emulsions, water-in-oil emulsions, mineral salts, polynucleotides, and natural substances. Specific adjuvants that may be used include Freund's adjuvant, oil such as MONTANIDE® ISA51, IL1, IL2, IL3, IL4, IL5, IL6, IL7, IL8, IL9, IL10, IL12, alpha-interferon, PTNGg, GM-CSF, GMCSP, BCG, LT-a, aluminum salts, such as aluminum hydroxide or other aluminum compound, MDP compounds, such as thur-MDP and nor-MDP, CGP (MTP-PE), lipid A, monophosphoryl lipid A (MPL), lipopeptides (e.g., Pam3Cys). RIBI, which contains three components extracted from bacteria, MPL, trehalose dimycolate (TDM), and cell wall skeleton (CWS) in a 2% squalene/Tween 80 emulsion. MHC antigens may even be used.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (CARBOPOL®) used as an about 0.25% solution, aggregation of the protein in the vaccine by heat treatment with temperatures ranging between about 700 to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (FLUOSOL-DA®) used as a block substitute may also be employed to produce an adjuvant effect.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as y-interferon, IL-2, or IL-12 or genes encoding proteins involved in immune helper functions, such as B-7.

Administration of the compositions described herein may be carried out via any of the accepted modes of administration of agents for serving similar utilities. In some aspects, a pharmaceutical composition described herein may be administered intravenously, intraarterially, subcutaneously, intradermally or intramuscularly. In specific aspects, the FimH RNA molecules and/or RNA-LNP compositions are administered intramuscularly. In certain aspects, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In one aspect, the pharmaceutical composition is formulated for intramuscular administration. In another aspect, the pharmaceutical composition is formulated for systemic administration, e.g., for intravenous administration.

Pharmaceutical compositions may be formulated into preparations in solid, semi-solid, liquid, lyophilized, frozen, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid and may be frozen or lyophilized. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration. In some aspects, when intended for oral administration, the pharmaceutical composition is in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, PRIMOJEL®, corn starch and the like; lubricants such as magnesium stearate or STEROTEX®; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. In some aspects, when intended for oral administration, compositions contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or excluded.

A liquid pharmaceutical composition, whether they be solutions, suspensions, or other like form, may include or exclude one or more of the following adjuvants: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation may be enclosed in ampoules, disposable syringes, or multiple dose vials made of glass or plastic. In one aspect, physiological saline is the adjuvant. In one aspect, an injectable pharmaceutical composition is sterile. A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection may be prepared by combining the nucleic acid or polypeptide with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The pharmaceutical compositions according to the present disclosure, or their pharmaceutically acceptable salts, are generally applied in a “therapeutically effective amount” or a “prophylactically effective amount” and in “a pharmaceutically acceptable preparation.” The term “pharmaceutically acceptable” refers to the non-toxicity of a material which does not interact with the action of the active component of the pharmaceutical composition. The terms “therapeutically effective amount” and “prophylactically effective amount” refer to the amount which achieves a desired reaction or a desired effect alone or together with further doses. In the case of the treatment of a particular disease, in one aspect, the desired reaction relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the individual parameters of the patient, including the age, body weight, general health, gender, and diet of the patient; the mode, time, and/or duration of administration; the rate of excretion; the drug combination; the severity of the particular disorder or condition; and the subject undergoing therapy. Accordingly, the doses administered of the compositions described herein may depend on various of such parameters. In the case that a reaction in a patient is insufficient with an initial dose, higher doses (or effectively higher doses achieved by a different, more localized route of administration) may be used. In some aspects, compositions (e.g., FimH RNA-LNP compositions) may be administered at dosage levels sufficient to deliver 0.0001 ng/μg/mg per kg to 100 ng/μg/mg per kg, 0.001 ng/μg/mg per kg to 0.05 ng/μg/mg per kg, 0.005 ng/μg/mg per kg to 0.05 ng/μg/mg per kg, 0.001 ng/μg/mg per kg to 0.005 ng/μg/mg per kg, 0.05 ng/μg/mg per kg to 0.5 ng/μg/mg per kg, 0.01 ng/μg/mg per kg to 50 ng/μg/mg per kg, 0.1 ng/μg/mg per kg to 40 ng/μg/mg per kg, 0.5 ng/μg/mg per kg to 30 ng/μg/mg per kg, 0.01 ng/μg/mg per kg to 10 ng/μg/mg per kg, 0.1 ng/μg/mg per kg to 10 ng/μg/mg per kg, or 1 ng/μg/mg per kg to 25 ng/μg/mg per kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). In some aspects, compositions (e.g., FimH RNA-LNP compositions) may be administered at dosage levels sufficient to deliver at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per kg, of subject body weight per day, one or more times a day, per week, per month, etc. to obtain the desired therapeutic, diagnostic, prophylactic, or imaging effect.

In some aspects, compositions (e.g., FimH RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per day, one or more times a day, per week, per month, etc. to obtain the desired therapeutic, diagnostic, prophylactic, or imaging effect.

In specific aspects, compositions (e.g., FimH RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL FimH RNA encapsulated in LNP.

In exemplary aspects, compositions (e.g., FimH RNA-LNP compositions) may be administered at dose levels of at least, at most, exactly, or between any two of 0.01, 0.15, 0.30, 0.45, 0.60, 0.75, or 0.90 mg/mL FimH RNA encapsulated in LNP. In exemplary aspects, compositions (e.g., FimH RNA-LNP compositions) may be administered at dose levels of at least, at most, exactly, or between any two of 0.01, 0.15, 0.30, 0.45, 0.60, 0.75, or 0.90 mg FimH RNA encapsulated in LNP.

In specific aspects, compositions (e.g., FimH RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL FimH RNA encapsulated in LNP.

In exemplary aspects, compositions (e.g., FimH RNA-LNP compositions) may be administered at dose levels of at least, at most, exactly, or between any two of 1, 15, 30, 45, 60, 75, or 90 μg/mL FimH RNA encapsulated in LNP. In exemplary aspects, compositions (e.g., FimH RNA-LNP compositions) may be administered at dose levels of at least, at most, exactly, or between any two of 1, 15, 30, 45, 60, 75, or 90 μg FimH RNA encapsulated in LNP.

The desired dosage may be delivered multiple times a day (e.g., 1, 2, 3, 4, 5, or more times a day), every other day, every third day, every week, every two weeks, every three weeks, every four weeks, every 2 months, every three months, every 6 months, etc. In certain aspects, the desired dosage may be delivered using a single-dose administration. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. The time of administration between the initial administration of the composition and a subsequent administration of the composition may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some aspects, compositions (e.g., FimH RNA-LNP compositions) may be administered in a single dose. In some aspects, compositions (e.g., FimH RNA-LNP compositions) may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 1 month later, Day 0 and 2 months later, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later), with each administration at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg FimH RNA encapsulated in LNP. Higher and lower dosages and frequency of administration are encompassed by the present disclosure. For example, compositions (e.g., FimH RNA-LNP compositions) may be administered three or four times. Periodic boosters at intervals of 1-5 years may be desirable to maintain protective levels of the antibodies.

In some aspects, the compositions (e.g., FimH RNA-LNP compositions) are administered to a subject as a single dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of FimH RNA encapsulated in LNP. In some aspects, the compositions (e.g., FimH RNA-LNP compositions) are administered the subject as a single dose of at least, at most, exactly, or between any two of 1 μg, 15 μg, 30 μg, 45 μg, 60 μg, 75 μg, or 90 μg of FimH RNA encapsulated in LNP.

In some aspects, the compositions (e.g., FimH RNA-LNP compositions) are administered to a subject as two doses of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of FimH RNA encapsulated in LNP. In some aspects, the compositions (e.g., FimH RNA-LNP compositions) are administered the subject as two doses of at least, at most, exactly, or between any two of 1 μg, 15 μg, 30 μg, 45 μg, 60 μg, 75 μg, or 90 μg of FimH RNA encapsulated in LNP.

In specific aspects, compositions (e.g., FimH RNA-LNP compositions) may be administered twice (e.g., Day 0 and Day 28, Day 0 and Day 60, Day 0 and Day 180, Day 0 and 2 months later, Day 0 and 6 months later), with each administration at a total dose of or at dosage levels sufficient to deliver a total dose of at least, at most, exactly, or between any two of 1 μg, 15 μg, 30 μg, 45 μg, 60 μg, 75 μg, or 90 μg FimH RNA encapsulated in LNP.

### IX. Methods of Use

Provided herein are compositions (e.g., pharmaceutical compositions comprising FimH RNA molecules and/or FimH RNA-LNPs), methods, kits and reagents for prevention and/or treatment of E. coli infection in humans and other mammals.

The RNA (e.g., mRNA) vaccines may be utilized in various settings depending on the prevalence of the infection or the degree or level of unmet medical need. The RNA vaccines may be utilized to treat and/or prevent E. coli infection of various genotypes, strains, and isolates. The RNA vaccines typically have superior properties in that they produce much larger antibody titers and produce responses earlier than commercially available anti-bacterial therapeutic treatments.

While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

There may be situations in which persons are at risk for infection with more than one E. coli antigen. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one E. coli antigen, a combination vaccine can be administered that includes RNA (e.g., mRNA) encoding at least one antigenic polypeptide protein (or antigenic portion thereof) of a first E. coli antigen, e.g. FimH or a fragment thereof, or organism and further includes RNA encoding at least one antigenic polypeptide protein (or antigenic portion thereof) of a second antigen. RNA (e.g., mRNA) can be co-formulated, for example, in a single lipid nanoparticle (LNP) or can be formulated in separate LNPs for co-administration.

FimH RNA compositions (e.g., FimH RNA-LNP compositions) may be used as prophylactic agents. They may be used in medicine to prevent and/or treat infectious disease. In exemplary aspects, the FimH RNA compositions are used to provide prophylactic protection from urinary tract infections (UTI). The FimH vaccines of the present disclosure may be particularly useful for prevention and/or treatment of immunocompromised and elderly patients to prevent or to reduce the severity and/or duration of E. coli infection.

In some aspects, the FimH RNA compositions (e.g., FimH RNA-LNP compositions) of the disclosure are administered to a subject (e.g., a mammalian subject, such as a human subject), and the RNA polynucleotides are translated in vivo to produce an antigenic polypeptide.

In some aspects, the FimH RNA compositions of the disclosure may be used to prime immune effector cells, for example, to activate peripheral blood mononuclear cells (PBMCs) ex vivo, which are then infused (re-infused) into a subject.

In some aspects, after administration of a FimH RNA molecule described herein, e.g., formulated as RNA-LNPs, at least a portion of the RNA is delivered to a target cell. In some aspects, at least a portion of the RNA is delivered to the cytosol of the target cell. In some aspects, the RNA is translated by the target cell to produce the polypeptide or protein it encodes. In some aspects, the target cell is a spleen cell. In some aspects, the target cell is an antigen presenting cell such as a professional antigen presenting cell in the spleen. In some aspects, the target cell is a dendritic cell or macrophage. RNA molecules such as RNA-LNPs described herein may be used for delivering RNA to such target cell. Accordingly, the present disclosure also relates to a method for delivering RNA to a target cell in a subject comprising the administration of the RNA-particles described herein to the subject.

In some aspects, the RNA is delivered to the cytosol of the target cell. In some aspects, the RNA is translated by the target cell to produce the polypeptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

In some aspects, nucleic acid compositions described herein, e.g., compositions comprising a FimH RNA-LNP are characterized by (e.g., when administered to a subject) sustained expression of an encoded polypeptide. For example, in some aspects, such compositions are characterized in that, when administered to a human, they achieve detectable polypeptide expression in a biological sample (e.g., serum) from such human and, in some aspects, such expression persists for a period of time that is at least at least 36 hours or longer, including, e.g., at least 48 hours, at least 60 hours, at least 72 hours, at least 96 hours, at least 120 hours, at least 148 hours, or longer.

In some aspects, the disclosure relates to a method of inducing an immune response in a subject. The method includes administering to the subject an effective amount of an RNA molecule, RNA-LNP and/or composition as described herein.

In another aspect, the disclosure relates to a method of vaccinating a subject. The method includes administering to the subject in need thereof an effective amount of an RNA molecule, RNA-LNP and/or composition described herein.

In another aspect, the disclosure relates to a method of treating or preventing a bacterial disease. The method includes administering to the subject an effective amount of an RNA molecule RNA-LNP and/or composition as described herein.

In another aspect, the disclosure relates to a method of treating or preventing or reducing the severity of an E. coli infection and/or illness caused by E. coli. The method includes administering to the subject an effective amount of an RNA molecule, RNA-LNP and/or composition as described herein.

In another aspect, the disclosure relates to a method of treating or preventing or reducing the severity of an infection in a subject by, for example, inducing an immune response to the infectious agent, e.g. E. coli, in the subject. In some aspects, the method includes administering a priming composition that includes an effective amount of an RNA molecule, RNA-LNP and/or composition described herein, and administering a booster composition including an effective amount of an RNA molecule, RNA-LNP and/or composition. In some aspects, the composition elicits an immune response including an antibody response. In some aspects, the composition elicits an immune response including a T cell response.

In another aspect, the disclosure relates to a method of treating or preventing or reducing the severity of an E. coli infection and/or illness caused by E. coli in a subject by, for example, inducing an immune response to E. coli FimH in the subject. In some aspects, the method includes administering a priming composition that includes an effective amount of an RNA molecule, RNA-LNP and/or composition described herein, and administering a booster composition including an effective amount of an RNA molecule RNA-LNP and/or composition as described herein. In some aspects, the composition elicits an immune response including an antibody response. In some aspects, the composition elicits an immune response including a T cell response.

The methods disclosed herein may involve administering to the subject a FimH RNA-LNP composition comprising at least one FimH RNA molecule having an open reading frame encoding at least one FimH antigenic polypeptide, thereby inducing in the subject an immune response specific to E. coli FimH antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine against E. coli. An “anti-antigenic polypeptide antibody” is a serum antibody the binds specifically to the antigenic polypeptide. In some aspects, the anti-antigenic polypeptide antibody titer in the subject is increased at least, at most, between any two of, or exactly 1, 2, 3, 4, 5, 6, 7, 8, 9, or 10 log following administration of the FimH RNA-LNP composition relative to anti-antigenic polypeptide antibody titer in a subject administered a prophylactically effective dose of a traditional composition against FimH.

The methods disclosed herein may involve administering to the subject a FimH RNA-LNP composition comprising at least one FimH RNA molecule having an open reading frame encoding at least one FimH antigenic polypeptide, thereby inducing in the subject an immune response specific to FimH antigenic polypeptide, wherein the immune response in the subject is equivalent to an immune response in a subject administered with a traditional composition against the FimH at least, at most, in between any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

In some aspects, the RNA molecule, RNA-LNP and/or composition is used as a vaccine. In some aspects, the RNA molecule, RNA-LNP and/or composition may be used in various therapeutic or prophylactic methods for preventing, treating or ameliorating of urinary tract infection, urosepsis, pyelonephritis or cystitis.

FimH RNA compositions may be administered prophylactically to healthy subjects or early in infection during the incubation phase or during active infection after onset of symptoms. In some aspects, the subject is immunocompetent. In some aspects, the subject is immunocompromised.

In some aspects, the RNA molecule, RNA-LNP and/or composition is administered in a single dose. In some aspects, a second, third or fourth dose may be given. In some aspects, the RNA molecule, RNA-LNP and/or composition is administered in multiple doses.

In some aspects, the RNA molecule, RNA-LNP and/or composition is administered intramuscularly (IM) or intradermally (ID).

The present disclosure further provides a kit comprising the RNA molecule, RNA-LNP, and/or composition.

In some aspects, the RNA molecule, RNA-LNP and/or composition described herein is administered to a subject that is less than about 1 years old, or about 1 years old to about 10 years old, or about 10 years old to about 20 years old, or about 20 years old to about 50 years old, or about 60 years old to about 70 years old, or older.

In some aspects the subject is at least, at most, exactly, or between any two of less than 1 year of age, greater than 1 year of age, greater than 5 years of age, greater than 10 years of age, greater than 20 years of age, greater than 30 years of age, greater than 40 years of age, greater than 50 years of age, greater than 60 years of age, greater than 70 years of age, or older. In some aspects, the subject is greater than 50 years of age.

In some aspects the subject is at least, at most, exactly, or between any two of about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject may be about 50 years of age or older.

In some aspects the subject is at least, at most, exactly, or between any two of 1 year of age or older, 5 years of age or older, 10 years of age or older, 20 years of age or older, 30 years of age or older, 40 years of age or older, 50 years of age or older, 60 years of age or older, 70 years of age or older, or older. In some aspects the subject may be 50 years of age or older.

In an embodiment, the mRNA vaccines of the invention comprise lipids. The lipids and modRNA can together form nanoparticles. The lipids can encapsulate the mRNA in the form of a lipid nanoparticle (LNP) to aid cell entry and stability of the RNA/lipid nanoparticles.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN® 20], polyoxy ethylene sorbitan [TWEEN® 60], polyoxy ethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T½ of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T½ of the LNP, LNP suspension, lyophilized LNP composition, or LNP formulation of the present disclosure is longer than the T½ of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T½” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

The RNA (e.g., mRNA) vaccines may be utilized in various settings depending on the prevalence of the infection or the degree or level of unmet medical need. The RNA vaccines may be utilized to treat and/or prevent E. coli infection of various genotypes, strains, and isolates. The RNA vaccines typically have superior properties in that they produce much larger antibody titers and produce responses earlier than commercially available anti-viral or anti-bacterial therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

There may be situations in which persons are at risk for infection with more than one E. coli antigen. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one E. coli antigen, a combination vaccine can be administered that includes RNA (e.g., mRNA) encoding at least one antigenic polypeptide protein (or antigenic portion thereof) of a first E. coli antigen, e.g. FimH or a fragment thereof, or organism and further includes RNA encoding at least one antigenic polypeptide protein (or antigenic portion thereof) of a second antigen. RNA (e.g., mRNA) can be co-formulated, for example, in a single lipid nanoparticle (LNP) or can be formulated in separate LNPs for co-administration.

Some embodiments of the present disclosure provide E. coli vaccines (or compositions or immunogenic compositions) that include at least one RNA polynucleotide having an open reading frame encoding at least one E. coli FimH antigenic polypeptide or an immunogenic fragment thereof (e.g., an immunogenic fragment capable of inducing an immune response to E. coli).

Some embodiments of the present disclosure provide E. coli vaccines that include at least one RNA polynucleotide having an open reading frame encoding at least one E. coli FimH polypeptide or an immunogenic fragment of the novel FimH polypeptide sequences described above (e.g., an immunogenic fragment capable of inducing an immune response to E. coli). In some embodiments, an E. coli vaccine comprises at least one RNA (e.g., mRNA) polynucleotide having an open reading frame encoding at least one FimH polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel FimH sequences described above. The modified sequence can be at least 75% (e.g., any number between 75% and 100%, inclusive, e.g., 70%, 80%, 85%, 90%, 95%, 99%, and 100%) identical to an amino acid sequence of the novel FimH sequences described above.

Some embodiments of the present disclosure provide an isolated nucleic acid comprising a sequence encoding the novel E. coli FimH polypeptide sequences described above; an expression vector comprising the nucleic acid; and a host cell comprising the nucleic acid. The present disclosure also provides a method of producing a polypeptide of any of the novel E. coli FimH sequences described above. A method may include culturing the host cell in a medium under conditions permitting nucleic acid expression of the novel E. coli FimH sequences described above, and purifying from the cultured cell or the medium of the cell a novel E. coli FimH polypeptide.

In some embodiments, a RNA (e.g., mRNA) vaccine further comprising an adjuvant.

In some embodiments, at least one RNA polynucleotide encodes at least one E. coli FimH polypeptide that does not attach to cells.

In some embodiments, at least one RNA polynucleotide encodes at least one E. coli FimH polypeptide that does not allow binding of the bacteria to a cell, wherein the cell is a bladder epithelial cell. Some embodiments of the present disclosure provide a vaccine that includes at least one ribonucleic acid (RNA) (e.g., mRNA) polynucleotide having an open reading frame encoding at least one E. coli FimH polypeptide, at least one 5′ terminal cap and at least one chemical modification, formulated within a lipid nanoparticle.

In some embodiments, a 5′ terminal cap is m7G(5′)ppp(5′)(2′OMeA)pG.

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

Some embodiments of the present disclosure provide a vaccine that includes at least one RNA (e.g., mRNA) polynucleotide having an open reading frame encoding at least one E. coli FimH polypeptide, wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of an RNA (e.g., mRNA) polynucleotide encodes at least one E. coli polypeptide. In a preferred embodiment, the E. coli polypeptide is a fimbrial antigen. In a preferred embodiment, the E. coli fimbrial antigen is FimH. In some embodiments, the open reading frame encodes at least two, at least five, or at least ten E. coli polypeptides. In some embodiments, the open reading frame encodes at least 100 E. coli polypeptides. In some embodiments, the open reading frame encodes 1-100 E. coli polypeptides.

In some embodiments, a vaccine comprises at least two RNA (e.g., mRNA) polynucleotides, each having an open reading frame encoding at least one E. coli FimH polypeptide. In some embodiments, the vaccine comprises at least five or at least ten RNA (e.g., mRNA) polynucleotides, each having an open reading frame encoding at least one E. coli FimH polypeptide or an immunogenic fragment thereof. In some embodiments, the vaccine comprises at least 100 RNA (e.g., mRNA) polynucleotides, each having an open reading frame encoding at least one E. coli FimH polypeptide. In some embodiments, the vaccine comprises 2-100 RNA (e.g., mRNA) polynucleotides, each having an open reading frame encoding at least one E. coli FimH polypeptide.

In a further aspect, the invention provides a multivalent vaccine, wherein the multivalent vaccine comprises at least two RNA (e.g., mRNA) polynucleotides, each having an open reading frame encoding at least one E. coli FimH polypeptide. In some embodiments, the multivalent vaccine comprises at least five or at least ten RNA (e.g., mRNA) polynucleotides, each having an open reading frame encoding at least one E. coli FimH polypeptide or an immunogenic fragment thereof. In some embodiments, the multivalent vaccine comprises at least 100 RNA (e.g., mRNA) polynucleotides, each having an open reading frame encoding at least one E. coli FimH polypeptide. In some embodiments, the multivalent vaccine comprises 2-100 RNA (e.g., mRNA) polynucleotides, each having an open reading frame encoding at least one E. coli FimH polypeptide. In a further embodiment, the multivalent vaccine comprises RNA (e.g., mRNA) polynucleotides, each having an open reading frame encoding at least one additional polypeptide including, but not limited to, E. coli FmIH, E. coli PapG, K. pneu. MrkA, E. faecalis EbpA, or immunogenic fragments thereof.

Also provided herein is an E. coli RNA (e.g., mRNA) vaccine of any one of the foregoing paragraphs formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol.

In some embodiments, the nanoparticle has a polydispersity value of less than 0.4 (e.g., less than 0.3, 0.2 or 0.1).

In some embodiments, the nanoparticle has a net neutral charge at a neutral pH value.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is an E. coli vaccine. In some embodiments, the RNA (e.g., mRNA) vaccine is a combination vaccine comprising a combination of E. coli vaccines (a broad-spectrum E. coli vaccine).

In some embodiments, an antigen-specific immune response comprises a T cell response or a B cell response.

In some embodiments, a method of producing an antigen-specific immune response comprises administering to a subject a single dose (no booster dose) of an E. coli RNA (e.g., mRNA) vaccine of the present disclosure.

In some embodiments, a method further comprises administering to the subject a second (booster) dose of an E. coli RNA (e.g., mRNA) vaccine. Additional doses (boosters) of an E. coli RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, an antigen can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an E. coli RNA (e.g., mRNA) vaccine is administered to a subject by intradermal injection, intramuscular injection, or by intranasal administration. In some embodiments, an E. coli RNA (e.g., mRNA) vaccine is administered to a subject by intramuscular injection.

Some embodiments, of the present disclosure provide methods of inducing an antigen specific immune response in a subject, including administering to a subject an E. coli RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an E. coli FimH polypeptide) following administration to the subject of any of the E. coli RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered an E. coli FimH polypeptide or fragment thereof, or wherein the control is an anti-antigenic polypeptide antibody titer produced in a subject who has been administered a recombinant or purified E. coli FimH vaccine. In some embodiments, the control is an anti-antigenic polypeptide antibody titer produced in a subject who has not been administered a recombinant or purified E. coli FimH vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant E. coli vaccine, wherein the anti-antigenic polypeptide antibody titer produced in the subject is equivalent to an anti-antigenic polypeptide antibody titer produced in a control subject administered the standard of care dose of a recombinant E. coli protein vaccine. In some embodiments, the effective amount is a dose equivalent to 2- to 1000-fold reduction in the standard of care dose of a recombinant E. coli protein vaccine, wherein the anti-antigenic polypeptide antibody titer produced in the subject is equivalent to an anti-antigenic polypeptide antibody titer produced in a control subject administered the standard of care dose of a recombinant E. coli protein vaccine.

In some embodiments, the RNA (e.g., mRNA) vaccine is formulated in an effective amount to produce an antigen specific immune response in a subject.

In some embodiments, the effective amount is a total dose <25 μg. In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two or more times. In some embodiments, the effective amount is a dose of 100 μg administered to the subject a total of two or more times. In some embodiments, the effective amount is a dose of 400 μg administered to the subject a total of two or more times. In some embodiments, the effective amount is a dose of 500 μg administered to the subject a total of two or more times.

In some embodiments, the efficacy (or effectiveness) of a RNA (e.g., mRNA) vaccine is greater than 60%. In some embodiments, the RNA (e.g., mRNA) polynucleotide of the vaccine encodes at least one E. coli FimH polypeptide.

Vaccine efficacy may be assessed using standard analyses. For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and Efficacy=(1−RR)×100.

Likewise, vaccine effectiveness may be assessed using standard analyses. Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against E. coli for up to 2 years. In some embodiments, the vaccine immunizes the subject against E. coli for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, an RNA (e.g., mRNA) vaccine may be administered later in life, for example, at the age of about 6 months to about 5 years, or older.

In some embodiments, the subject is an adolescent between the ages of about 11-19 years (e.g., about 11, 12, 13, 14, 15, 16, 17, 18, or 19 years old).

In some embodiments, the subject is an adult between the ages of about 20 years and about 59 years (e.g., about 20, 25, 30, 35, 40, 45, 50, 55 or 59 years old).

In some embodiments, the subject is an older adult subject about 60 years old, about 70 years old, or older (e.g., about 60, 65, 70, 75, 80, 85 or 90 years old).

In some embodiments, the subject has been exposed to E. coli; the subject is infected with E. coli; or subject is at risk of infection by E. coli.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first antigenic polypeptide, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is <1 μg, 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40- 300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of bacteria binding to the plate.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no modified nucleotides, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 μg and 400 μg of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a bacteria or virus in an individual or population of individuals comprising administering to said individual or population an antigenic memory booster nucleic acid vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP-formulated RNA polynucleotide having an open reading frame comprising no nucleotide modifications (unmodified), the open reading frame encoding a first antigenic polypeptide, wherein the vaccine has at least 10-fold less RNA polynucleotide than is required for an unmodified mRNA vaccine not formulated in a LNP to produce an equivalent antibody titer. In some embodiments, the RNA polynucleotide is present in a dosage of 25-100 micrograms.

Both chemically modified and unmodified RNA vaccines are useful according to the invention. Prior art reports that it is preferable to use chemically unmodified mRNA formulated in a carrier for the production of vaccines. Both the chemically modified and unmodified RNA vaccines of the invention produce better immune responses than mRNA vaccines formulated in a different lipid carrier.

In other aspects the invention encompasses a method of treating an older adult subject age 60 years or older comprising administering to the subject a nucleic acid vaccine comprising one or more RNA polynucleotides having an open reading frame encoding an E. coli antigenic polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding an E. coli antigenic polypeptide in an effective amount to vaccinate the subject.

In other aspects the invention encompasses a method of treating an adult subject between the ages of about 20 years and about 50 years old comprising administering to the subject a nucleic acid vaccine comprising one or more RNA polynucleotides having an open reading frame encoding an E. coli antigenic polypeptide in an effective amount to vaccinate the subject.

In some aspects, the invention is a method of vaccinating a subject with a combination vaccine including at least two nucleic acid sequences encoding antigens wherein the dosage for the vaccine is a combined therapeutic dosage wherein the dosage of each individual nucleic acid encoding an antigen is a subtherapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject.

In preferred aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in a subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA) or Luminex. In exemplary embodiments, antibody titer is determined or measured by neutralization assay, e.g., by E. coli binding inhibition assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose). In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml antigen-specific antibodies. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose). In exemplary embodiments, antibody level or concentration is determined or measured by enzyme-linked immunosorbent assay (ELISA) or Luminex. In exemplary embodiments, antibody level or concentration is determined or measured by neutralization assay, e.g., by E. coli binding inhibition assay.

### EXAMPLES

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for. The following Examples illustrate some embodiments of the invention.

### Example 1

### RNA-Based Expression of E. coli FimH Antigens in Mammalian Cells

Expression of FimH in mammalian cells has been described in International Patent Publication NO. WO2021084429A1, which is hereby incorporated by reference in its entirety. FimH mutations that stabilize protein conformation and improve bioprocessing properties related to expression and purification, as well as functional immunogenicity have been described in International Patent Publication No. WO2022090893, which is hereby incorporated by reference in its entirety. Preclinical efficacy data from a cynomolgous macaque cystitis challenge model show that a recombinant full length FimH-DSG triple mutant (G15A G16A V27A) protein is protective when adminstered with a liposomal MPLA/QS21 adjuvant and is described in International Patent Publication No. WO2022137078, which is hereby incorporated by reference in its entirety. Expression of FimH from an mRNA vaccine may provide the benefit of improved cellular immunity, as well as lower cost of goods relative to a protein antigen. It is unknown whether a cell-associated construct expressed on the cell surface or a secreted FimH will be more immunogenic in vivo. Therefore different constructs were generated with these properties and the results are set forth hereinbelow.

In the following Example we evaluated the expression of chimeric FimH antigens designed to localize to the cell surface by anchoring to the plasma membrane. For this, we fused the FimH lectin domain comprising stabilizing triple mutations to C-terminal transmembrane domains of herpes simplex virus gD (HSVgD), SARS-COV2 or the C-terminal GPI-anchor signal of human decay-accelerating factor (DAF) protein (also known as CD55) (DAFgpi). We also tested whether a secreted construct could be generated by engineering a construct for extracellular expression of our benchmark FimH-DSG triple mutant (G15A G16A V27A) (N-deglycosylated), which has previously been shown to be efficiently secreted from mammalian cells following transfection with DNA expression plasmids.

### Materials and Methods

### 1. Plasmid Construction and Cloning and In Vitro Transcription

DNA sequences encoding E. coli FimH proteins were prepared and utilized for in vitro transcription reactions to generate RNA. In vitro transcription of RNA is known in the art and is described herein. DNA templates were cloned into a modRNA cloning entry vector with backbone sequence elements (T7 promoter, 5′ and 3′ UTR, 3′ 80 nt poly-A tail) for improved RNA stability and translational efficiency. The DNA was purified, spectrophotometrically quantified and in vitro-transcribed. CleanCap® AG kit (TriLink) was used which simultaneous caps newly transcribed mRNA molecules with m7G(5′)ppp(5′)(2′OMeA)pG.

The FimH RNA was generated from codon-optimized (CO) DNA for stabilization and superior protein expression. Table 4 shows RNA constructs of the present disclosure, and corresponding sequences, comprising a 5′ UTR, an open reading frame encoding a FimH polypeptide, a 3′ UTR and a poly-A tail.

### 2. Immunofluorescence Analysis of Transfected HeLa Cells

HeLa cells (ATCC #CCL-2) were seeded at 1×10⁴ cells/well in a polylysine coated 96-well plate. RNA was diluted to 100 ng/well and 50 ng/well in media, combined with diluted lipofectamine (MessengerMax, Invitrogen) for 5 min at room temperature (RT). Next, the mRNA plus lipofectamine mixture was added to each well in triplicate. The microplate was centrifuged at 500 g for 5 min at RT and incubated overnight at 37° C. and 5% CO₂. The next day the monolayers were rinsed in PBS with Ca²⁺/Mg²⁺ and cell surfaces stained with 1 μg/mL of anti-FimH mAb 926 (reconstructed from International Publication No. WO2016183501, which is hereby incorporated by reference in its entirety) in 3% BSA in PBS with Ca²⁺/Mg²⁺ for 1 hr at 4° C. For total cellular staining cells were fixed with 4% PFA for 20 min at RT, then permeabilized with 0.1% Saponin in 0.1% goat serum. Binding of the FimH antibody was detected with a secondary goat anti-human IgG conjugated to AlexaFluor488 at 1:500 final dilution (Invitrogen). Nuclei were stained with 4′6-Diamidino-2-Phenylindole, Dilactate (DAPI) at 100 ng/mL. CellMask Orange Stain (ThermoFisher) was used (at 1:140,000 final dilution) as a cell delineation tool for automated high content analysis by labeling the entire cell. Confocal imaging of stained cells was done using the Opera Phenix Plus High-Content Screening platform (PerkinElmer). Automated quantitation of cellular fluorescence was done using the Harmony® imaging software. Complete cells within each captured image were identified by a combination of nuclear staining (DAPI) and cytoplasmic staining (CellMask). Images from 250 fields per well under 40× magnification were captured and analyzed. Cells from mock transfections delineated in this way were used to define baseline threshold values for quantifying protein antigen fluorescence intensity (MFI) in both total & surface-stained cells.

### 3. Flow Cytometry and Octet Analysis of Transfected Expi293 Cells

25 μL volumes of 5-fold serial dilutions of RNA (from 500 ng/well) were combined with lipofectamine (MessengerMax, Invitrogen) for 5 min RT in a 96-well deep-well (2.2 mL) plate. Expi293 suspension cells (ThermoFisher) were diluted in 0.45 mL of Opti-MEM growth media to a final concentration of 1×10⁶/well with shaking for 24 h at 37° C. 8% CO₂ and 80% humidity overnight. The next day cells were divided equally into different 96-well plates to perform surface and total staining as described for the HeLa cells except that the FimH mAb was used at a concentration of 5.0 μg/ml. Cells were also stained with Fixable Dye eFluor® 780 to assess cell viability. Plates were read on an LSRII flow cytometer instrument (BD Biosciences).

FimH secreted into culture media supernatants 24 hour after the transfection of Expi293 cells with the FimH-DSG-TM mRNA were quantitated by Octet biolayer interferometry. Transfections with 5-fold dilutions of RNA from 500 ng/well were evaluated. Anti-human Fc biosensors were used to first bind the FimH specific mAb followed by binding reactions with clarified FimH transfection supernatants. The biosensors were first hydrated with conditioned Expi293 media (200 μL/well) for 10 min at RT, followed by capture with saturating concentration of mAb (final 10 μg/mL, 200 μL/well) for 10 minutes at room temperature on an orbital shaker. FimH concentrations were determined by interpolating values from a parallel titration of purified recombinant FimH-DSG standard using linear regression analysis.

### Results and Discussion

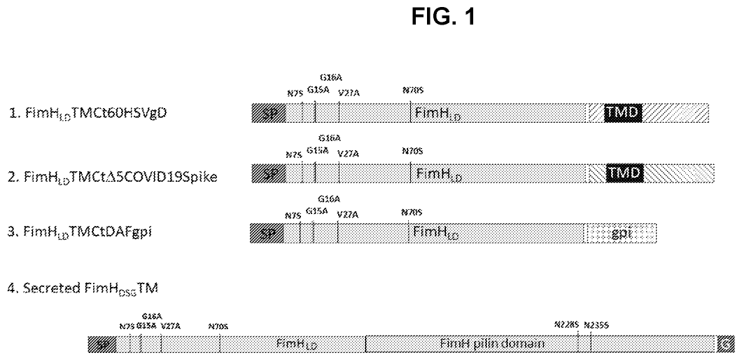

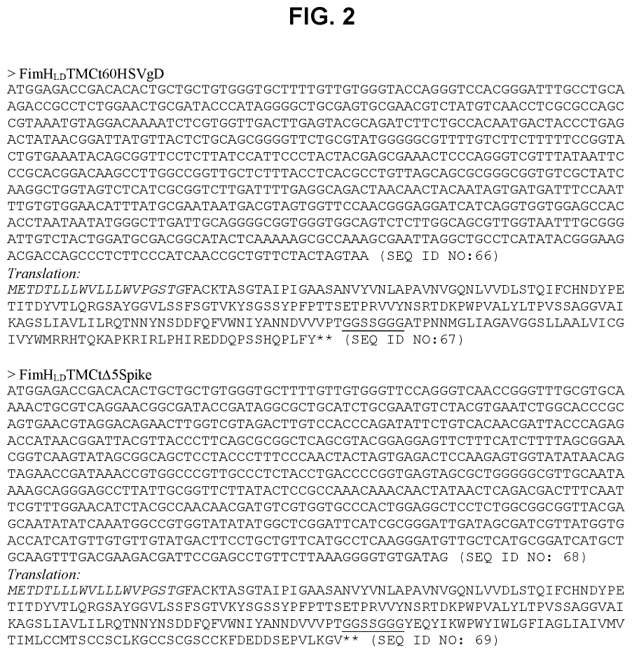

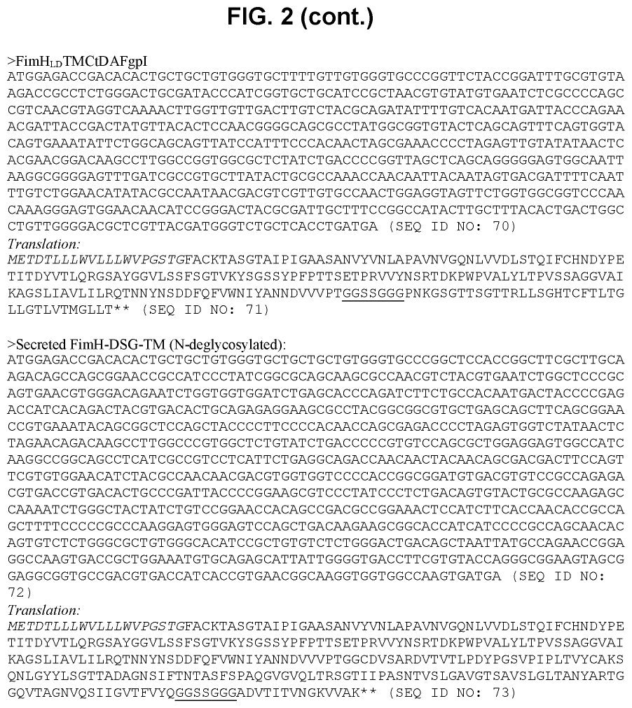

Expression of bacterial FimH in mammalian cells to expose the ligand binding site in its native conformation requires processing by mammalian signal peptidase in a manner that precisely recapitulates processing by the analogous bacterial signal peptidase prior to secretion into the E. coli periplasmic space. The signal peptide used for mammalian expression is the same mouse IgG kappa sequence previously shown to confer efficient secretion of biologically active E. coli FimH in mammalian cells (see International Publication No. WO2022/090893) (SEQ ID NO: 65). Gene chimeras of the FimH lectin domain harboring the G15A G16A V27A triple mutation (TM) were fused to C-terminal membrane targeting domains of three different viral glycoproteins, separated by a 7 amino acid linker sequence “GGSSGGG” (SEQ ID NO: 74). The gene for the secreted FimH-DSG™ was also cloned into the mRNA vector and evaluated. Structural features of these FimH proteins are highlighted in FIG. 1. Gene and protein sequences are listed in FIG. 2, and Tables 1-3.

The four constructs and their key features are summarized as follows:

### 1) FimHLDTMCt60HSVgD:

FimH_{LD}TM fused to the trimeric C-terminal domain comprising the Ct-60 amino acid residues of HSV gD including a hydrophobic transmembrane domain (TMD) (Cocchi F, et al. Proceedings of the National Academy of Sciences of the United States of America 2004; 101:7445);

2) FimH_{LD}TMCtΔ5Spike (Also Known as “FimH_{LD}TMCtΔ5COVID19Spike”):

FimH_{LD}TM fused to the trimeric transmembrane membrane domain of the SARS-CoV2 Spike protein which spans the C-terminal 68 amino acid residues. The C-terminal 5 amino acids were deleted, as they constitute an endoplasmic reticulum (ER) retention motif (KLHYT) (SEQ ID NO: 80) that reduces intracellular trafficking efficiency (Xia X. Viruses 2021; 13:109). The C-terminal domain includes eight TM-proximal amino acids including juxtamembrane aromatic residues. Immediately distal to the hydrophobic transmembrane domain (TMD) is a conserved cysteine-rich region (residues 1234-1254 of SEQ ID NO: 79) which contains multiple palmitoylation sites. In SARS-CoV-1, palmitoylation at those sites facilitates membrane partitioning and cell fusion (McBride C E, Machamer C E. Virology 2010; 405:139-48). The C-terminal cytoplasmic tail also contains a conserved charged region (1255-KFDEDDSE (SEQ ID NO: 81) (Xia X. Viruses 2021; 13:109);

### 3) FimHLDTMCtDAFgpi:

FimH_{LD}TM fused to the membrane targeting domain of the monomeric human DAF protein gpi-anchor signal. It includes the C-terminal 37 residues sufficient for conferring heterologous membrane association on heterologous viral glycoprotein ectodomains (Lisanti M P, et al. Journal of Cell Biology 1989; 109:2145-56). The bulk of C-terminal GPI-attachment signal is cleaved off in the endoplasmic reticulum concomitantly with addition of the glycosylphosphatidylinositol (GPI, also know as “gpi” or “gpl”) lipid moiety (Galian C, et al. J Biol Chem 2012; 287:16399-409); and

### 4) Secreted FimH-DSG-TM (N-Deglycosylated):

The secreted fully N-deglycosylated (aglycosyl) version of the FimH-DSG™ with pilin domain asparagines that are ordinarily N-glycosylated substituted with serines (N228S N235S) in order to prevent glycosylation. The C-terminal donor strand G-peptide added is appended to the FimH protein C-terminus, separated by the seven amino acid linker sequence “GGSSGGG” (SEQ ID NO: 74).

Sequences of the above-described FimH constructs, including the antigens/polypeptides, DNA and RNA of the present invention are provided in Tables 1-3, respectively. The sequences may comprise any stop codon.

TABLE 1
FimH Construct Polypeptides

| | | SEQ ID |
|---|---|---|
| ID | Sequence | NO: |
| FimHLDTMCt | METDTLLLWVLLLWVPGSTGFACKTASGTAIPIGAASANVYVNLAP | 67 |
| 60HSVgD | AVNVGQNLVVDLSTQIFCHNDYPETITDYVTLQRGSAYGGVLSSF | |
| Mouse IgK | SGTVKYSGSSYPFPTTSETPRVVYNSRTDKPWPVALYLTPVSSAG | |
| signal peptide | GVAIKAGSLIAVLILRQTNNYNSDDFQFVWNIYANNDVVVPTGGSS | |
| (italics) | GGGATPNNMGLIAGAVGGSLLAALVICGIVYWMRRHTQKAPKRIR | |
| Interdomain | LPHIREDDQPSSHQPLFY** | |
| linker | | |
| (underlined); | | |
| Stop codons | | |
| (asterisks) | | |
| FimH_{LD}TMCt | METDTLLLWVLLLWVPGSTGFACKTASGTAIPIGAASANVYVNLAP | 69 |
| Δ5Spike | AVNVGQNLVVDLSTQIFCHNDYPETITDYVTLQRGSAYGGVLSSF | |
| Mouse IgK | SGTVKYSGSSYPFPTTSETPRVVYNSRTDKPWPVALYLTPVSSAG | |
| signal peptide | GVAIKAGSLIAVLILRQTNNYNSDDFQFVWNIYANNDVVVPTGGSS | |
| (italics) | GGGYEQYIKWPWYIWLGFIAGLIAIVMVTIMLCCMTSCCSCLKGCC | |
| Interdomain | SCGSCCKFDEDDSEPVLKGV** | |
| linker | | |
| (underlined); | | |
| Stop codons | | |
| (asterisks) | | |
| FimHLDTMCt | METDTLLLWVLLLWVPGSTGFACKTASGTAIPIGAASANVYVNLAP | 71 |
| DAFgpl | AVNVGQNLVVDLSTQIFCHNDYPETITDYVTLQRGSAYGGVLSSF | |
| Mouse IgK | SGTVKYSGSSYPFPTTSETPRVVYNSRTDKPWPVALYLTPVSSAG | |
| signal peptide | GVAIKAGSLIAVLILRQTNNYNSDDFQFVWNIYANNDVVVPTGGSS | |
| (italics) | GGGPNKGSGTTSGTTRLLSGHTCFTLTGLLGTLVTMGLLT** | |
| Interdomain | | |
| linker | | |
| (underlined); | | |
| Stop codons | | |
| (asterisks) | | |
| Secreted | METDTLLLWVLLLWVPGSTGFACKTASGTAIPIGAASANVYVNLAP | 73 |
| FimH-DSG- | AVNVGQNLVVDLSTQIFCHNDYPETITDYVTLQRGSAYGGVLSSF | |
| TM (N- | SGTVKYSGSSYPFPTTSETPRVVYNSRTDKPWPVALYLTPVSSAG | |
| deglycosylated) | GVAIKAGSLIAVLILRQTNNYNSDDFQFVWNIYANNDVVVPTGGCD | |
| Mouse IgK | VSARDVTVTLPDYPGSVPIPLTVYCAKSQNLGYYLSGTTADAGNSI | |
| signal peptide | FTNTASFSPAQGVGVQLTRSGTIIPASNTVSLGAVGTSAVSLGLTA | |
| (italics) | NYARTGGQVTAGNVQSIIGVTFVYQGGSSGGGADVTITVNGKVVA | |
| Interdomain | K** | |
| linker | | |
| (underlined); | | |
| Stop codons | | |
| (asterisks) | | |

TABLE 2
FimH Construct DNA

| | | SEQ ID |
|---|---|---|
| ID | Sequence | NO: |
| FimHLDTMCt | ATGGAGACCGACACACTGCTGCTGTGGGTGCTTTTGTTGTGGG | 66 |
| 60HSVgD | TACCAGGGTCCACGGGATTTGCCTGCAAGACCGCCTCTGGAA | |
| DNA | CTGCGATACCCATAGGGGCTGCGAGTGCGAACGTCTATGTCAA | |
| (Amino acid | CCTCGCGCCAGCCGTAAATGTAGGACAAAATCTCGTGGTTGAC | |
| SEQ ID NO: 67) | TTGAGTACGCAGATCTTCTGCCACAATGACTACCCTGAGACTAT | |
| | AACGGATTATGTTACTCTGCAGCGGGGTTCTGCGTATGGGGGC | |
| | GTTTTGTCTTCTTTTTCCGGTACTGTGAAATACAGCGGTTCCTC | |
| | TTATCCATTCCCTACTACGAGCGAAACTCCCAGGGTCGTTTATA | |
| | ATTCCCGCACGGACAAGCCTTGGCCGGTTGCTCTTTACCTCAC | |
| | GCCTGTTAGCAGCGCGGGCGGTGTCGCTATCAAGGCTGGTAG | |
| | TCTCATCGCGGTCTTGATTTTGAGGCAGACTAACAACTACAATA | |
| | GTGATGATTTCCAATTTGTGTGGAACATTTATGCGAATAATGAC | |
| | GTAGTGGTTCCAACGGGAGGATCATCAGGTGGTGGAGCCACA | |
| | CCTAATAATATGGGCTTGATTGCAGGGGCGGTGGGTGGCAGT | |
| | CTCTTGGCAGCGTTGGTAATTTGCGGGATTGTCTACTGGATGC | |
| | GACGGCATACTCAAAAAGCGCCAAAGCGAATTAGGCTGCCTCA | |
| | TATACGGGAAGACGACCAGCCCTCTTCCCATCAACCGCTGTTC | |
| | TACTAGTAA | |
| FimH_{LD}TMCt | ATGGAGACCGACACACTGCTGCTGTGGGTGCTTTTGTTGTGGG | 68 |
| Δ5Spike | TTCCAGGGTCAACCGGGTTTGCGTGCAAAACTGCGTCAGGAAC | |
| DNA | GGCGATACCGATAGGCGCTGCATCTGCGAATGTCTACGTGAAT | |
| (Amino acid | CTGGCACCCGCAGTGAACGTAGGACAGAACTTGGTCGTAGACT | |
| SEQ ID NO: 69) | TGTCCACCCAGATATTCTGTCACAACGATTACCCAGAGACCATA | |
| | ACGGATTACGTTACCCTTCAGCGCGGCTCAGCGTACGGAGGA | |
| | GTTCTTTCATCTTTTAGCGGAACGGTCAAGTATAGCGGCAGCT | |
| | CCTACCCTTTCCCAACTACTAGTGAGACTCCAAGAGTGGTATAT | |
| | AACAGTAGAACCGATAAACCGTGGCCCGTTGCCCTCTACCTGA | |
| | CCCCGGTGAGTAGCGCTGGGGGCGTTGCAATAAAAGCAGGGA | |
| | GCCTTATTGCGGTTCTTATACTCCGCCAAACAAACAACTATAAC | |
| | TCAGACGACTTTCAATTCGTTTGGAACATCTACGCCAACAACGA | |
| | TGTCGTGGTGCCCACTGGAGGCTCCTCTGGCGGCGGTTACGA | |
| | GCAATATATCAAATGGCCGTGGTATATATGGCTCGGATTCATCG | |
| | CGGGATTGATAGCGATCGTTATGGTGACCATCATGTTGTGTTG | |
| | TATGACTTCCTGCTGTTCATGCCTCAAGGGATGTTGCTCATGC | |
| | GGATCATGCTGCAAGTTTGACGAAGACGATTCCGAGCCTGTTC | |
| | TTAAAGGGGTGTGATAG | |
| FimHLDTMCt | ATGGAGACCGACACACTGCTGCTGTGGGTGCTTTTGTTGTGGG | 70 |
| DAFgpl | TGCCCGGTTCTACCGGATTTGCGTGTAAGACCGCCTCTGGGAC | |
| DNA | TGCGATACCCATCGGTGCTGCATCCGCTAACGTGTATGTGAAT | |
| (Amino acid | CTCGCCCCAGCCGTCAACGTAGGTCAAAACTTGGTTGTTGACT | |
| SEQ ID NO: 71) | TGTCTACGCAGATATTTTGTCACAATGATTACCCAGAAACGATT | |
| | ACCGACTATGTTACACTCCAACGGGGCAGCGCCTATGGCGGT | |
| | GTACTCAGCAGTTTCAGTGGTACAGTGAAATATTCTGGCAGCA | |
| | GTTATCCATTTCCCACAACTAGCGAAACCCCTAGAGTTGTATAT | |
| | AACTCACGAACGGACAAGCCTTGGCCGGTGGCGCTCTATCTGA | |
| | CCCCGGTTAGCTCAGCAGGGGGAGTGGCAATTAAGGCGGGGA | |
| | GTTTGATCGCCGTGCTTATACTGCGCCAAACCAACAATTACAAT | |
| | AGTGACGATTTTCAATTTGTCTGGAACATATACGCCAATAACGA | |
| | CGTCGTTGTGCCAACTGGAGGTAGTTCTGGTGGCGGTCCCAA | |
| | CAAAGGGAGTGGAACAACATCCGGGACTACGCGATTGCTTTCC | |
| | GGCCATACTTGCTTTACACTGACTGGCCTGTTGGGGACGCTCG | |
| | TTACGATGGGTCTGCTCACCTGATGA | |
| Secreted | ATGGAGACCGACACACTGCTGCTGTGGGTGCTGCTGCTGTGG | 72 |
| FimH-DSG- | GTGCCCGGCTCCACCGGCTTCGCTTGCAAGACAGCCAGCGGA | |
| TM (N- | ACCGCCATCCCTATCGGCGCAGCAAGCGCCAACGTCTACGTG | |
| deglycosylated) | AATCTGGCTCCCGCAGTGAACGTGGGACAGAATCTGGTGGTG | |
| DNA | GATCTGAGCACCCAGATCTTCTGCCACAATGACTACCCCGAGA | |
| (Amino acid | CCATCACAGACTACGTGACACTGCAGAGAGGAAGCGCCTACG | |
| SEQ ID NO: 73) | GCGGCGTGCTGAGCAGCTTCAGCGGAACCGTGAAATACAGCG | |
| | GCTCCAGCTACCCCTTCCCCACAACCAGCGAGACCCCTAGAGT | |
| | GGTCTATAACTCTAGAACAGACAAGCCTTGGCCCGTGGCTCTG | |
| | TATCTGACCCCCGTGTCCAGCGCTGGAGGAGTGGCCATCAAG | |
| | GCCGGCAGCCTCATCGCCGTCCTCATTCTGAGGCAGACCAAC | |
| | AACTACAACAGCGACGACTTCCAGTTCGTGTGGAACATCTACG | |
| | CCAACAACGACGTGGTGGTCCCCACCGGCGGATGTGACGTGT | |
| | CCGCCAGAGACGTGACCGTGACACTGCCCGATTACCCCGGAA | |
| | GCGTCCCTATCCCTCTGACAGTGTACTGCGCCAAGAGCCAAAA | |
| | TCTGGGCTACTATCTGTCCGGAACCACAGCCGACGCCGGAAA | |
| | CTCCATCTTCACCAACACCGCCAGCTTTTCCCCCGCCCAAGGA | |
| | GTGGGAGTCCAGCTGACAAGAAGCGGCACCATCATCCCCGCC | |
| | AGCAACACAGTGTCTCTGGGCGCTGTGGGCACATCCGCTGTG | |
| | TCTCTGGGACTGACAGCTAATTATGCCAGAACCGGAGGCCAAG | |
| | TGACCGCTGGAAATGTGCAGAGCATTATTGGGGTGACCTTCGT | |
| | GTACCAGGGCGGAAGTAGCGGAGGCGGTGCCGACGTGACCAT | |
| | CACCGTGAACGGCAAGGTGGTGGCCAAGTGATGA | |

TABLE 3
FimH Construct RNA

| | | SEQ ID |
|---|---|---|
| ID | Sequence | NO: |
| FimHLDTMCt | AUGGAGACCGACACACUGCUGCUGUGGGUGCUUUUGUUGUGGGUA | 82 |
| 60HSVgD | CCAGGGUCCACGGGAUUUGCCUGCAAGACCGCCUCUGGAACUGCG | |
| RNA | AUACCCAUAGGGGCUGCGAGUGCGAACGUCUAUGUCAACCUCGCG | |
| (Amino acid | CCAGCCGUAAAUGUAGGACAAAAUCUCGUGGUUGACUUGAGUACGC | |
| SEQ ID NO: 67) | AGAUCUUCUGCCACAAUGACUACCCUGAGACUAUAACGGAUUAUGU | |
| | UACUCUGCAGCGGGGUUCUGCGUAUGGGGGCGUUUUGUCUUCUUU | |
| | UUCCGGUACUGUGAAAUACAGCGGUUCCUCUUAUCCAUUCCCUACU | |
| | ACGAGCGAAACUCCCAGGGUCGUUUAUAAUUCCCGCACGGACAAGC | |
| | CUUGGCCGGUUGCUCUUUACCUCACGCCUGUUAGCAGCGCGGGCG | |
| | GUGUCGCUAUCAAGGCUGGUAGUCUCAUCGCGGUCUUGAUUUUGA | |
| | GGCAGACUAACAACUACAAUAGUGAUGAUUUCCAAUUUGUGUGGAA | |
| | CAUUUAUGCGAAUAAUGACGUAGUGGUUCCAACGGGAGGAUCAUCA | |
| | GGUGGUGGAGCCACACCUAAUAAUAUGGGCUUGAUUGCAGGGGCG | |
| | GUGGGUGGCAGUCUCUUGGCAGCGUUGGUAAUUUGCGGGAUUGUC | |
| | UACUGGAUGCGACGGCAUACUCAAAAAGCGCCAAAGCGAAUUAGGC | |
| | UGCCUCAUAUACGGGAAGACGACCAGCCCUCUUCCCAUCAACCGCU | |
| | GUUCUACUAGUAA | |
| FimH_{LD}TMCt | AUGGAGACCGACACACUGCUGCUGUGGGUGCUUUUGUUGUGGGUU | 83 |
| Δ5Spike | CCAGGGUCAACCGGGUUUGCGUGCAAAACUGCGUCAGGAACGGCG | |
| RNA | AUACCGAUAGGCGCUGCAUCUGCGAAUGUCUACGUGAAUCUGGCAC | |
| (Amino acid | CCGCAGUGAACGUAGGACAGAACUUGGUCGUAGACUUGUCCACCCA | |
| SEQ ID NO: 69) | GAUAUUCUGUCACAACGAUUACCCAGAGACCAUAACGGAUUACGUU | |
| | ACCCUUCAGCGCGGCUCAGCGUACGGAGGAGUUCUUUCAUCUUUU | |
| | AGCGGAACGGUCAAGUAUAGCGGCAGCUCCUACCCUUUCCCAACUA | |
| | CUAGUGAGACUCCAAGAGUGGUAUAUAACAGUAGAACCGAUAAACC | |
| | GUGGCCCGUUGCCCUCUACCUGACCCCGGUGAGUAGCGCUGGGGG | |
| | CGUUGCAAUAAAAGCAGGGAGCCUUAUUGCGGUUCUUAUACUCCGC | |
| | CAAACAAACAACUAUAACUCAGACGACUUUCAAUUCGUUUGGAACAU | |
| | CUACGCCAACAACGAUGUCGUGGUGCCCACUGGAGGCUCCUCUGG | |
| | CGGCGGUUACGAGCAAUAUAUCAAAUGGCCGUGGUAUAUAUGGCUC | |
| | GGAUUCAUCGCGGGAUUGAUAGCGAUCGUUAUGGUGACCAUCAUG | |
| | UUGUGUUGUAUGACUUCCUGCUGUUCAUGCCUCAAGGGAUGUUGC | |
| | UCAUGCGGAUCAUGCUGCAAGUUUGACGAAGACGAUUCCGAGCCU | |
| | GUUCUUAAAGGGGUGUGAUAG | |
| FimHLDTMCt | AUGGAGACCGACACACUGCUGCUGUGGGUGCUUUUGUUGUGGGUG | 84 |
| DAFgpl | CCCGGUUCUACCGGAUUUGCGUGUAAGACCGCCUCUGGGACUGCG | |
| RNA | AUACCCAUCGGUGCUGCAUCCGCUAACGUGUAUGUGAAUCUCGCCC | |
| (Amino acid | CAGCCGUCAACGUAGGUCAAAACUUGGUUGUUGACUUGUCUACGCA | |
| SEQ ID NO: 71) | GAUAUUUUGUCACAAUGAUUACCCAGAAACGAUUACCGACUAUGUU | |
| | ACACUCCAACGGGGCAGCGCCUAUGGCGGUGUACUCAGCAGUUUC | |
| | AGUGGUACAGUGAAAUAUUCUGGCAGCAGUUAUCCAUUUCCCACAA | |
| | CUAGCGAAACCCCUAGAGUUGUAUAUAACUCACGAACGGACAAGCC | |
| | UUGGCCGGUGGCGCUCUAUCUGACCCCGGUUAGCUCAGCAGGGGG | |
| | AGUGGCAAUUAAGGCGGGGAGUUUGAUCGCCGUGCUUAUACUGCG | |
| | CCAAACCAACAAUUACAAUAGUGACGAUUUUCAAUUUGUCUGGAACA | |
| | UAUACGCCAAUAACGACGUCGUUGUGCCAACUGGAGGUAGUUCUG | |
| | GUGGCGGUCCCAACAAAGGGAGUGGAACAACAUCCGGGACUACGC | |
| | GAUUGCUUUCCGGCCAUACUUGCUUUACACUGACUGGCCUGUUGG | |
| | GGACGCUCGUUACGAUGGGUCUGCUCACCUGAUGA | |
| Secreted | AUGGAGACCGACACACUGCUGCUGUGGGUGCUGCUGCUGUGGGUG | 85 |
| FimH-DSG- | CCCGGCUCCACCGGCUUCGCUUGCAAGACAGCCAGCGGAACCGCC | |
| TM (N- | AUCCCUAUCGGCGCAGCAAGCGCCAACGUCUACGUGAAUCUGGCUC | |
| deglycosylated) | CCGCAGUGAACGUGGGACAGAAUCUGGUGGUGGAUCUGAGCACCC | |
| RNA | AGAUCUUCUGCCACAAUGACUACCCCGAGACCAUCACAGACUACGU | |
| (Amino acid | GACACUGCAGAGAGGAAGCGCCUACGGCGGCGUGCUGAGCAGCUU | |
| SEQ ID NO: 73) | CAGCGGAACCGUGAAAUACAGCGGCUCCAGCUACCCCUUCCCCACA | |
| | ACCAGCGAGACCCCUAGAGUGGUCUAUAACUCUAGAACAGACAAGC | |
| | CUUGGCCCGUGGCUCUGUAUCUGACCCCCGUGUCCAGCGCUGGAG | |
| | GAGUGGCCAUCAAGGCCGGCAGCCUCAUCGCCGUCCUCAUUCUGA | |
| | GGCAGACCAACAACUACAACAGCGACGACUUCCAGUUCGUGUGGAA | |
| | CAUCUACGCCAACAACGACGUGGUGGUCCCCACCGGCGGAUGUGA | |
| | CGUGUCCGCCAGAGACGUGACCGUGACACUGCCCGAUUACCCCGG | |
| | AAGCGUCCCUAUCCCUCUGACAGUGUACUGCGCCAAGAGCCAAAAU | |
| | CUGGGCUACUAUCUGUCCGGAACCACAGCCGACGCCGGAAACUCCA | |
| | UCUUCACCAACACCGCCAGCUUUUCCCCCGCCCAAGGAGUGGGAGU | |
| | CCAGCUGACAAGAAGCGGCACCAUCAUCCCCGCCAGCAACACAGUG | |
| | UCUCUGGGCGCUGUGGGCACAUCCGCUGUGUCUCUGGGACUGACA | |
| | GCUAAUUAUGCCAGAACCGGAGGCCAAGUGACCGCUGGAAAUGUGC | |
| | AGAGCAUUAUUGGGGUGACCUUCGUGUACCAGGGCGGAAGUAGCG | |
| | GAGGCGGUGCCGACGUGACCAUCACCGUGAACGGCAAGGUGGUGG | |
| | CCAAGUGAUGA | |

TABLE 4
FimH modRNA constructs

| | | | | Poly-A | DNA/ |
|---|---|---|---|---|---|
| | 5′-UTR | ORF | 3′-UTR | tail* | Protein |
| | SEQ | SEQ | SEQ | SEQ | SEQ |
| RNA Construct | ID NO | ID NO | ID NO | ID NO | ID NO |
| FimHLDTMCt60HSVgD | 75 | 82 | 76 | 86 | 66/67 |
| FimH_{LD}TMCtΔ5Spike | 75 | 83 | 76 | 86 | 68/69 |
| FimHLDTMCtDAFgpI | 75 | 84 | 76 | 86 | 70/71 |
| Secreted FimH-DSG- | 75 | 85 | 76 | 86 | 72/73 |
| TM (N-deglycosylated) | | | | | |

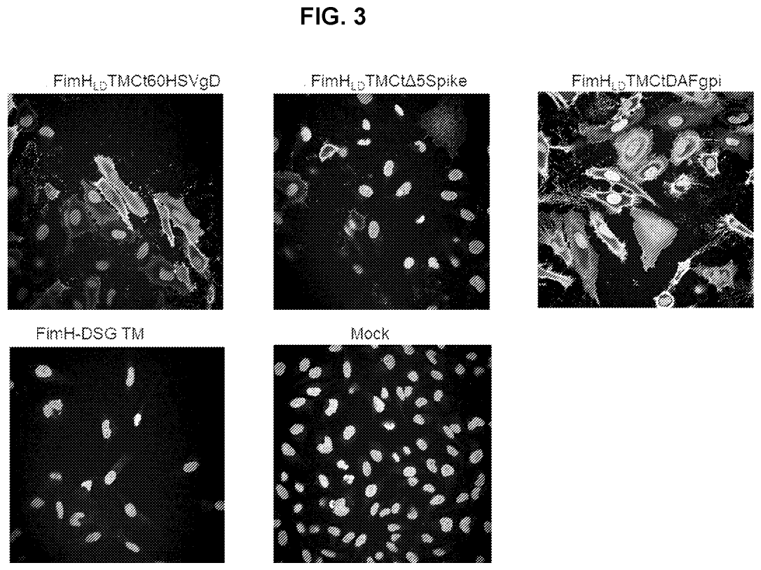

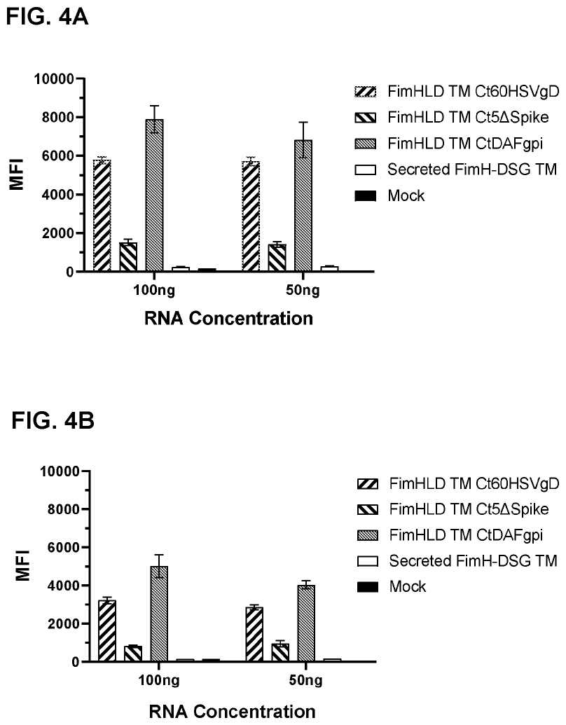

Expression of FimH from mRNAs transfected into adherent HeLa cells was evaluated by confocal fluorescence microscopy and the results are shown in FIG. 3, FIG. 4A and FIG. 4B. Microscope images of cells fixed and stained with FimH antibody (FIG. 3) show that the C-terminal GPI-anchor domain of the human DAF protein mediates efficient targeting of the antigen to the external surface of the HeLa cell membrane. These 37 amino acids harbor a C-terminal hydrophobic domain and a cleavage/attachment site that directs the proteolytic removal of the C-terminal 28 residues and attachment of the lipidated GPI anchor to the acceptor serine located only eight residues from the boundary with the “GGSSGGG” (SEQ ID NO: 74) linker separating this domain from FimH_{LD} (Moran P, Caras I W. J Cell Biol 1991; 115:329-36). Relatively low levels of FimH were observed with the SARS-COV2 C-terminal Spike protein chimera, while intermediate levels were detected with the C-terminal HSVgD chimera. No background level expression at all was seen with mock-transfected cells. Negligible levels of surface expression were observed with the mRNA expressing the secreted FimH-DSG antigen, which was expected based on efficient secretion observed previously with plasmid DNA expression vectors (see International Publication No. WO2022/090893). Similar patterns of expression for all mRNA constructs were observed with permeabilized HeLa cells compared with non-permeabilized fixed cells (FIG. 4A and FIG. 4B), suggesting that most of the FimH is exposed on the HeLa cell surface.

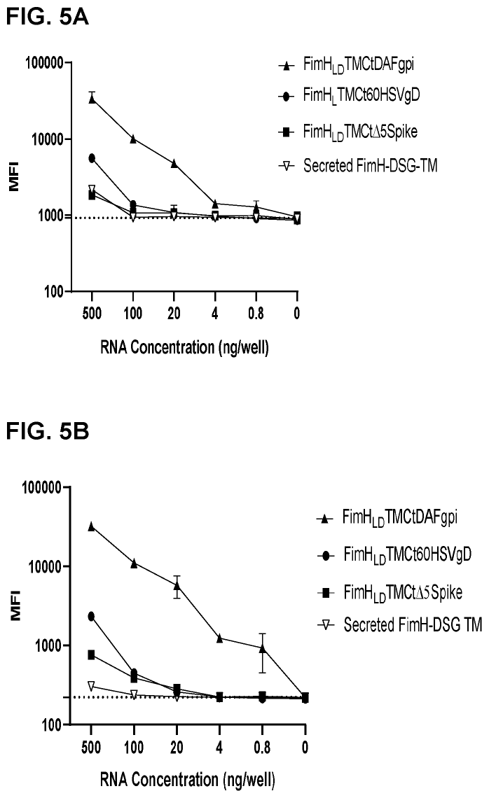

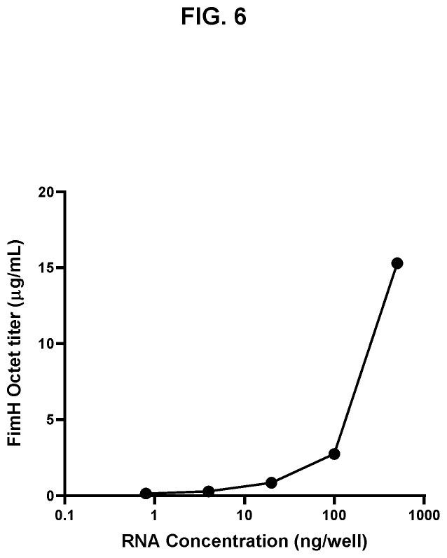

The same mRNAs were transfected into Expi293 suspension cells for parallel assessment of FimH antigen targeting in a different mammalian cell type. Results shown in FIG. 5A and FIG. 5B are consistent with the results observed with transfected adherent HeLa cells. The chimeric FimH with the C-terminal GPI-anchor yielded the highest levels of expression, titratable down to 20 ng of transfected mRNA. The FimH chimera with the C-terminal HSVgD transmembrane domain expressed well only at the 500 ng level, while the C-terminal Spike protein chimera expressed relatively poorly. Culture supernatants from the transfections were initially analyzed for levels of secreted protein by Western blot with robust levels of antigen detected only for transfections with the FimH-DSG antigen (data not shown). In this case biolayer interferometry confirmed the presence of FimH antigen proportional to the concentration of RNA transfected at 20 ng, 100 ng and 500 ng levels (FIG. 6).

Results demonstrate that the FimH_{LD} antigen can be expressed on the mammalian cell surface by fusing heterologous membrane targeting signals to the C-terminus. These chimeric fusion proteins are expressed from transfected mRNAs in both HeLa and Epi293 cells. Membrane targeting signals can be derived from either viral glycoproteins or from a GPI-anchor signal. Further optimization of membrane targeted expression of these prototypic FimH-viral-glycoprotein chimeras is possible, such as the addition of fibritin trimerization (Foldon) motif, used to promote trimerization of recombinant viral glycoprotein antigens (Vogel A B, Kanevsky I, Che Y, et al. Nature 2021; 592:283-9; Meier S, Guthe S, Kiefhaber T, Grzesiek S. J Mol Biol 2004; 344:1051-69). We also confirmed in these experiments, as observed previously with transfected plasmid DNA expression vectors, that the full length FimH-DSG protein can be efficiently secreted into the cell culture media when expressed from transfected mRNA.

### Example 2

Immunogenicity in Mice of modRNA LNPs Expressing Escherichia coli FimH Antigens

In Example 1 it was shown that secreted and membrane-targeted forms of the Escherichia coli fimbrial antigen FimH can be expressed in mammalian cells in vitro from transfected mRNAs. In this Example 2, we assessed the ability of modRNA lipid nanoparticles (LNPs) to elicit functional neutralizing antibodies in mice. The LNPs generated FimH IgG and neutralizing antibody titers that were significantly more robust than recombinant FimH protein formulated with LiNA-2 adjuvant. As used herein “LiNA-2” shall mean a liposomal adjuvant comprising MPLA and QS21. FimH-specific Th1 and Th2 responses were determined in splenocytes of vaccinated animals. A flow-cytometry-based intracellular cytokine staining (ICS) assay was used to measure the production and accumulation of cytokines and surface expression of activation-induced markers (AIM) upon stimulation of T-cells with a FimH peptide library. The modRNAs elicted a stronger CD8+ T cell response compared to the protein/LiNA2 antigen. Vaccination with the membrane-targeted FimH modRNAs resulted in an increased FimH-specific Th1-biased CD4+ T cell response compared to the protein/LiNA2 antigen or the modRNA expressing the secreted FimH_{DSG}TM. These results support the use of the described FimH modRNAs as vaccines in the treatment of UTI.

### Materials and Methods

### 1. Animal Study Details

CD-1 female mice from Charles River Lab (6-8 weeks old upon arrival) were immunized with protein or LNP antigens at 7-9 weeks old. The study schedule was as follows:
(i) vaccinations were administered at weeks 0, 4, and 8;
(ii) animals were bled at weeks 0, 3, 6 and exsanguinated at week 10; and
(iii) spleens were harvested from 5 mice in each group at week 10 (terminal timepoint).

Dosing and vaccine components are summarized in Table 5 and Table 6.

TABLE 5
Mouse Study EC-088

| | | | Vaccine | | | Adjuvant | Delivery |
|---|---|---|---|---|---|---|---|
| Group | Animals | Antigen | dose | | Adjuvant | Dose | (volume) |
| 1 | 15 | Aglycosyl-FimH_{DSG} | 10 μg prime/ | | LiNA-2 | 10 μg | IM |
| | | | 5 μg boost | | | PHAD | (0.05 mL) |
| | | | | | | (MPLA)/ | |
| | | | | | | 5 μg | |
| | | | | | | QS-21 | |
| 2 | 20 | FimH_{DSG} (secreted) | 10 | μg | — | — | IM |
| | | LNP | | | | | (0.05 mL) |
| 3 | 20 | FimH_{DSG} (secreted) | 1 | μg | — | — | IM |
| | | LNP | | | | | (0.05 mL) |
| 4 | 20 | FimH_{LD}-gpi LNP | 10 | μg | — | — | IM |
| | | | | | | | (0.05 mL) |
| 5 | 20 | FimH_{LD}-gpi LNP | 1 | μg | — | — | IM |
| | | | | | | | (0.05 mL) |

TABLE 6
Vaccine Components

| Vaccine Component | | Concentration |
|---|---|---|
| Antigen | Aglycosyl FimH_{DSG} | 0.84 mg/mL |
| | protein | |
| Antigen | Secreted FimH_{DSG}TM | 0.193 mg/mL |
| | (N-deglycosylated) LNP | |
| Antigen | FimH_{LD}-TMCtDAFgpi | 0.203 mg/mL |
| | LNP | |
| Adjuvant | LiNA-2 | 0.4 mg/mL QS21/0.8 |
| | | mg/mL 3D PHAD (MPLA) |

### 2. ModRNA LNP Production

Construction of plasmid mRNA vectors with four FimH gene variants harboring the conformation stabilizing triple mutations (G15A G16A V27A) was previously described in Example 1 set forth hereinabove. FimH genes encoding a full-length secreted FimH_{DSG} protein or membrane targeted FimH_{LD} chimeras were cloned between 5′ and 3′ UTRs. Two of these surface membrane targeting constructs, were not progressed to immunogenicity studies based on lower levels of FimH antigen expression observed in in vitro expression (IVE) experiments with HeLa or Expi293 mammalian cells transfected with the FimH mRNAs. The two best performing constructs prioritized for LNP formulation were the secreted full-length FimH_{DSG}TM (N-deglycosylated) (FIG. 1), and a membrane-associated FimH_{LD} gpi-anchored chimera (FimH_{LD}-TMCtDAFgpi) (FIG. 1). Properties of the two modRNA plasmids used to generate in vitro RNA transcripts and LNPs are summarized in Table 7.

TABLE 7
Plasmid Construct Details

| Plasmid | Construct Details | Description |
|---|---|---|
| pBVT_CF3 | pDT_A09_FimH_{LD} | FimH_{LD}TM fused to the membrane targeting domain of |
| | TM CtDAFgpi | the monomeric human DAF protein gpi anchor signal |
| pBVT_CF4 | pDT_A09_FimH_{DSG}- | The secreted fully N-deglycosylated version of the |
| | TM N7S N70S | FimH-DSG TM (G15A G16A V27A) with pilin domain |
| | N228S N235S (fully | asparagines substituted with serines (N228S N235S) |
| | deglycosylated) | |

In vitro transcription from plasmid DNA templates linearized with restriction enzyme BspQI was done using T7-polymerase and CleanCap® reagents (TriLink Biotechnologies). The resulting RNAs contain a 5′ Cap structure with pseudouridine incorporated instead of uridine. Transcripts were capped and purity of the three mRNAs was found to be >92% by fragment analyzer analysis and the capping efficiency was determined to be >97%.

RNAs were formulated into LNP formulations comprising 2 functional lipids, ALC-0315 and ALC-0159, and 2 structural lipids DSPC (1,2distearoyl-sn-glycero-3-phosphocholine) and cholesterol. The physicochemical properties and the structures of the 4 lipids are shown in the Table 8 below.

Lipid nanoparticles were prepared and tested according to the general procedures described in U.S. Pat. No. 9,737,619 (PCT Pub. No. WO2015/199952) and U.S. patent Ser. No. 10/166,298 (WO 2017/075531) and WO2020/146805, each of which is hereby incorporated by reference in its entirety. Briefly, cationic lipid (ALC-0315), cholesterol, DSPC, and PEG-lipid (ALC-0159) were solubilized in ethanol at a molar ratio of about 46.3:42.7:9.4:1.6.

TABLE 8
Lipids in the LNP Formulation

| | Molecular | Molecular Formula | |
|---|---|---|---|
| Lipid (CAS | Weight | Physical state and | |
| number) | [Da] | Strorage condition | Chemical name and structure |
| Cationic Lipid ALC-0315 (Not applicable) | 766 | C₄₈H₉₅NO₅ Liquid (oil) −20° C. | (4-hydroxybutyl)azanediyl)bis(hexane-6,1- diyl)bis(2-hexyldecanoate) 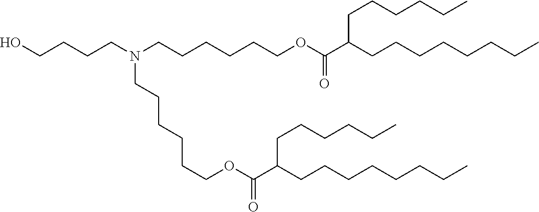 [SMILES: CCCCCCCCC(CCCCCC)C(=O)OCCCCCCN(CCCCO)CCCCCCOC(=O)C(CCCCCC)CCCCCCCC] |
| PEG-Lipid ALC-0159 (1849616-42-7) | ~2400- 2600 | C₃₀H₆₀NO(C₂H₄O)ₙOCH₃ n = 45-50 Solid −20° C. | 2-[(polyethylene glycol)-2000]-N,N- ditetradecyclacetamide 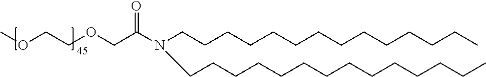 |
| DSPC (816-94-4) | 790 | C₃₀H₈₈NO₈P Solid −20° C. | 1,2-Distearoyl-sn-glycero-3-phosphocholine 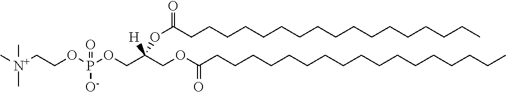 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol (57-88-5) | 387 | C₂₇H₄₆O Solid −20° C. | 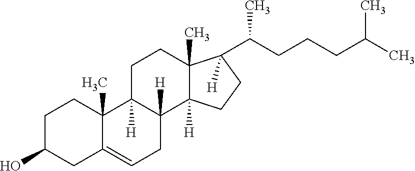 |

CAS = Chemical Abstract Service; DSPC = 1,2-disteroyl-sn-glycero-3-phosphocholine

RNA integrity was assessed by fragment analyzer capillary gel electrophoresis, encapsulation efficiency was assessed by RiboGreen assay, LNP size and polydispersity index (PDI) was assessed by dynamic light scattering (DLS) (Malvern) and endotoxin was assessed by the LAL test cartridge system (Endosafe). Properties of the antigens are summarized in Table 9.

TABLE 9
LNP Quality Attributes

| | | | | | | | DLS | | |
|---|---|---|---|---|---|---|---|---|---|
| | Encapsulation efficiency | | | | | | (Malvern) | | |
| | Total | Free | % RNA | RNA integrity | | | Size | | |
| Sample | RG RNA | RG RNA | encaps. | % POI | % LMMS | % LMS | (nm) | PDI | Endotoxin |
| FimH_{LD}- | 0.203 | 0.006 | 97 | 95.0 | 4.6 | 0.3 | 54.4 | 0.19 | <2 EU/mL |
| TMCtDAFgpi | | | | | | | | | |
| Secreted | 0.193 | 0.006 | 97 | 91.9 | 7.1 | 0.9 | 54.7 | 0.17 | <2 EU/mL |
| FimH_{DSG}TM | | | | | | | | | |
| (N- | | | | | | | | | |
| deglycosylated) | | | | | | | | | |

### 3. Immunogenicity Assays

The FimH direct Luminex immunoassay (dLIA) IgG assay measures the binding of mouse serum antibodies to the FimH_{DSG} antigen, immobilized on Luminex bead microspheres with EDC/NHS. Beads were incubated with serially diluted individual mouse sera or FimH control mAb with shaking at 4° C. for 18 hours. After washing, bound FimH-specific IgG was detected with a PE-conjugated goat anti-mouse IgG mouse secondary antibody (90 minutes RT incubation). Microplates were read on FlexMap 3D instrument (Biorad). A FimH-specific mouse IgG mAb was used as an internal standards to quantify anti-FimH IgG levels. The mAb standard curve yielded a linear slope profile across 103 serum dilutions (log luminescence vs log serum dilution).

### 4. Live Bacterial FimH-Specific Neutralization Assay

For the yeast mannan assay black microtiter 96-well plates (Maxisorb, Nunc) were coated with 20 μg/ml of yeast mannan (Sigma-Aldrich) in PBS buffer. The wells were blocked with 1% bovine serum albumin (BSA, Sigma-Aldrich) in PBS for 20 min. The human bladder epithelial cell line 5637 was obtained from ATCC (ATCC HTB-9). Cells were grown on black tissue-culture microplates (Greiner) in RPMI 1640 (Sigma, St Louis, MO) supplemented with 10% fetal bovine serum (FBS; Sigma), 2.0 g/I sodium bicarbonate (Sigma) and 0.3 g/I I-glutamine, grown at 37° C. with 5% CO2 and utilized between passages 10 and 24. Surface expression of Uroplakin 1a receptor was confirmed by immunofluorescence staining with polyclonal antibody (Novus #NBP214694). E. coli serotype 025b UTI strain PFEEC0547 (Atlas UTI strain 1525121) was serially passaged in 10 mL static LB cultures at 37° C. to induce FimH expression. Expression of FimH on the bacterial surface was confirmed by flow cytometry with rabbit immune serum to FimH_{L}_antigen. Specificity of bacterial binding to mannan or bladder cells was established by the inclusion of negative control compound Methyl α-D-mannopyranoside (Sigma) which reduced binding by >95% at 50 mM levels. Eight-step two-fold serial dilutions of test sera starting at 1:100 (in PBS, 0.1% BSA) were co-incubated with 1×10⁷ E. coli for 1 h at 37° C. before adding to immobilized yeast mannan or 5637 cell monolayers. Serially diluted anti-FimH_{LD} rabbit serum was used as an internal standard on each plate. Plates were incubated for 1 h at 37° C. before washing away unbound bacteria. Bound E. coli were stained for 45 min at RT with 3 μg/ml of an 025b-specific mAb conjugated to Alexafluor 488. The mAb was reconstructed from variable light and heavy chain sequences of 025b antibody 3E9-11 as previously described (Szijarto V, et al. 2015. Antimicrob Agents Chemother 59:3109-3116). The human pTT5 IgG expression plasmid was used as cloning vector (NRC, Canada). The fluorescence Intensity of individual wells was read on a ClarioStar Plus instrument. IC₅₀ inhibition values were interpolated using sigmoidal dose response variable-slope curve fitting (Graphpad Prism). Titers are the reciprocal of the serum dilution at which half-maximal inhibition is observed. A vaccine antigen responder was defined as a neutralizing titer that exceeds 80% inhibition at the starting serum dilution of 1:100. In addition, the serum dilution titration of binding activity must satisfy variable slope sigmoidal curve fitting parameters (R²>0.95, with interpolated Log IC50 value or trigger an experimental repeat with a broader dilution until resolved). The statistical significance (p-value) of differences in responses between groups was determined using an unpaired t-test with Welch's correction applied to log-transformed data.

### 5. Adaptive Immune Response Profiling

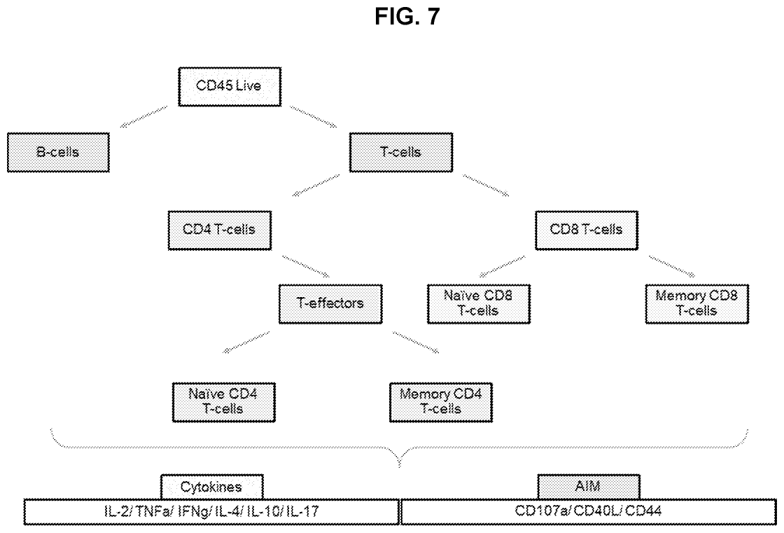

The flow cytometry gating strategy and fluorophore-labeled marker-specific antibodies used in the ICS assay are shown in FIG. 7 and Table 10. FimH-specific T cell responses were analyzed in freshly isolated splenocytes with a flow cytometry based intracellular cytokine staining (ICS) assay. The ICS assay compares the media-DMSO unstimulated response to the response observed in splenocytes after stimulation with a FimH peptide library.

In brief, mouse spleens were processed to obtain single cell suspensions using a non-enzymatic procedure (gentleMACS™). Spleens were subjected to red blood cell lysis and passaged through cell strainers to remove red blood cells and clumps. Splenocytes (2*10⁶ cells/well) were cultured in vitro in cRPMI with media-DMSO (unstimulated) or a FimH specific peptide library (15aa, 11aa overlap, 1 μg/mL/peptide, JPT peptide technologies, Berlin), for 6-7 hours at 37° C. in the presence of anti-CD107a APC antibody and protein transport inhibitors, GolgiPlug and GolgiStop. Following stimulation, splenocytes were incubated with fluorescently-conjugated antibodies to the surface proteins CD19, CD3, CD4, CD8, CD44, CD40L (25±5 minutes at 18-25° C.) followed by fixation and permeabilization and staining for intracellular proteins IFN-g, TNF-α, IL-2, IL-4, IL-10, IL-17 (25±5 minutes at 18-25° C.). After staining, the cells were washed and resuspended in Staining Buffer. Samples are acquired on a Cytek Aurora flow cytometer. Results are background (media-DMSO) subtracted and shown as percentage of cytokine-expressing CD4+ T cells and CD8+ T cells, respectively.

TABLE 10
Murine ICS/AIM Panel

| Specificity | Fluorophore | Purpose |
|---|---|---|
| CD19 | PerCp-Cy5.5 | B-cell, Exclusion |
| CD3 | BV605 | T-cells |
| CD4 | BV650 | CD4+ T-cells |
| CD8 | APC-H7 | CD8+ T-cells |
| CD107a | APC | Degranulation |
| IFNγ | Pe-CF594 | TH1 |
| TNFa | Pe-Cy7 | TH1 |
| IL2 | APC-R700 | TH1 |
| IL4 | BV711 | TH2 |
| IL 17A | FITC | TH17 |
| IL-10 | PE | Regulatory |
| CD40L | BV786 | Activation |
| CD44 | BV421 | Activation |

### Results and Discussion

Structural features of the FimH variants that were expressed as modRNAs in vitro and formulated as LNPs for this mouse immunogenicity study are illustrated in FIG. 1. The processed FimH_{DSG} protein expressed by the modRNA LNP has the same amino acid sequence as the purified recombinant protein antigen used as comparator in this study. The sequence includes conformation stabilizing triple mutations (G15A G16A V27A) and substitution mutations replacing asparagine N-glycosylation sites (N75S, N70S, N228S and N235S). The membrane-targeted FimH_{LD} chimeras possess the same mouse IgK signal peptide but lack the FimH C-terminal pilin domain.

### 1. FimH LNP Expression in Primary Human Skeletal Muscle Cells

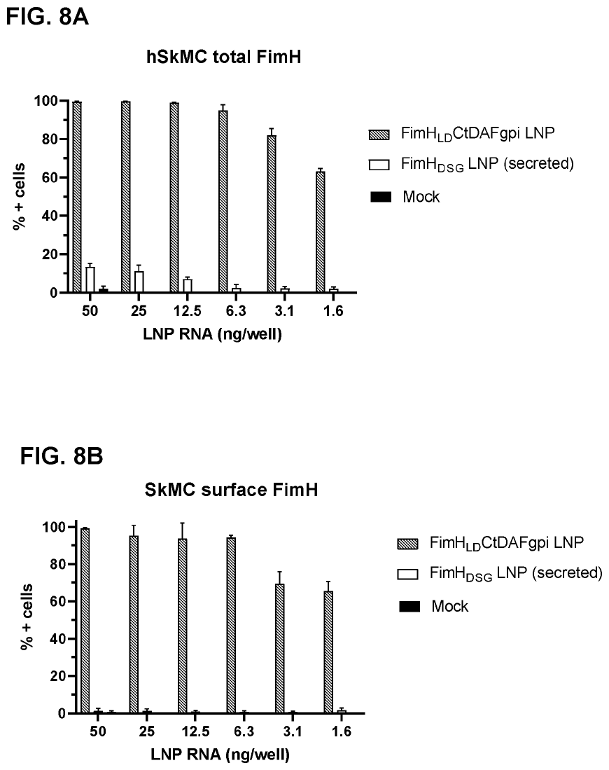

Expression of membrane-associated FimH following transfection of the modRNA LNPs was evaluated in primary human skeletal muscle cells cultivated in vitro. Total cellular FimH expression was assessed by fixing and permeabilizing the cells with paraformaldehyde and saponin prior to incubation with the FimH detection antibody. Surface staining was determined following paraformaldehyde fixation only. As shown in FIG. 8A and FIG. 8B, transfections with the FimH_{LD}-gpi chimera at levels of RNA greater than 1.6 ng per well resulted similar robust levels of FimH expression regardless of the fixation procedure, confirming that the antigen is predominantly exposed on the outer membrane surface. The FimH_{DSG} modRNA LNP served as a negative control, as previous mRNA transfection experiments (described in Example 1) showed that this antigen is efficiently secreted into the culture media. In this case, no surface expression was detected and only very low levels of intracellular expression was observed.

### 2. Mouse Immunogenicity

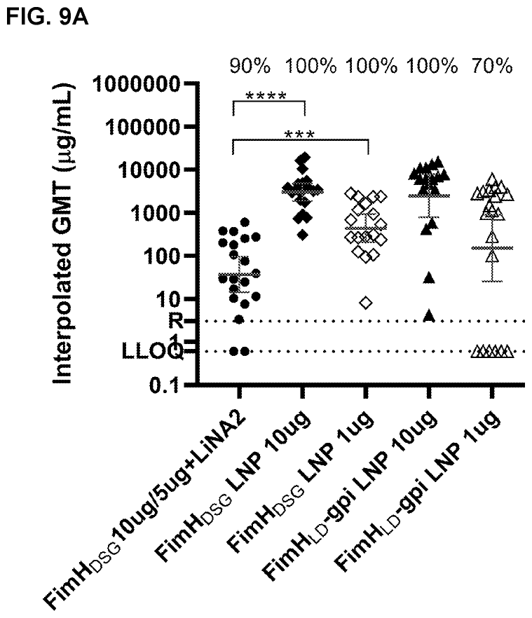

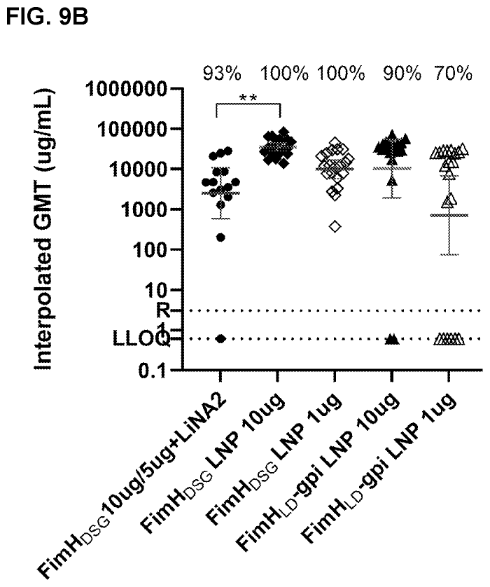

Levels of FimH specific IgG generated after vaccination of mice with adjuvanted FimH_{DSG} protein and the two LNPs are shown in FIGS. 9A and 9B and Table 11. Following a single vaccination with the FimH_{DSG} modRNA LNP at either dose level, IgG titers were significantly higher than after a single 10 μg dose of adjuvanted FimH_{DSG} protein. IgG titers for the modRNA LNPs trended higher at the 10 μg vs the 1 μg dose level but titers between the two different RNA-encoded antigens at either dose level were not significantly different. IgG titers increased 5-10 fold for all groups after a second dose of vaccine antigen. At this post-dose 2 (PD2) timepoint, titers for the 10 μg FimH_{DSG} encoded modRNA group were significantly higher than for the comparative FimH_{DSG} protein subunit antigen (after a second 5 μg dose) but trends toward higher titers for the other modRNA LNP groups were not statistically significant.

TABLE 11
FimH IgG dLIA Titer Summary

| | IgG GMT | | Responder | |
|---|---|---|---|---|
| | (μg/mL) | | Rate (%) | |
| | W3 | W6 | W3 | W6 |
| Antigen | (PD1) | (PD2) | (PD1) | (PD2) |
| FimH_{DSG}10 μg/5 μg + LiNA2 | 37 | 2512 | 90 | 93 |
| Secreted FimH_{DSG}TM LNP 10 μg | 3032 | 34914 | 100 | 100 |
| Secreted FimH_{DSG}TM LNP 1 μg | 438 | 9874 | 100 | 100 |
| FimH_{LD}-TMCtDAFgpi LNP 10 μg | 2492 | 10311 | 100 | 90 |
| FimH_{LD}-TMCtDAFgpi LNP 1 μg | 151 | 706 | 70 | 70 |

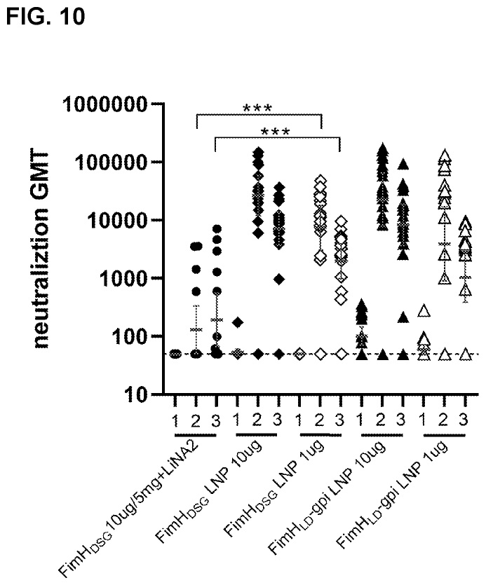

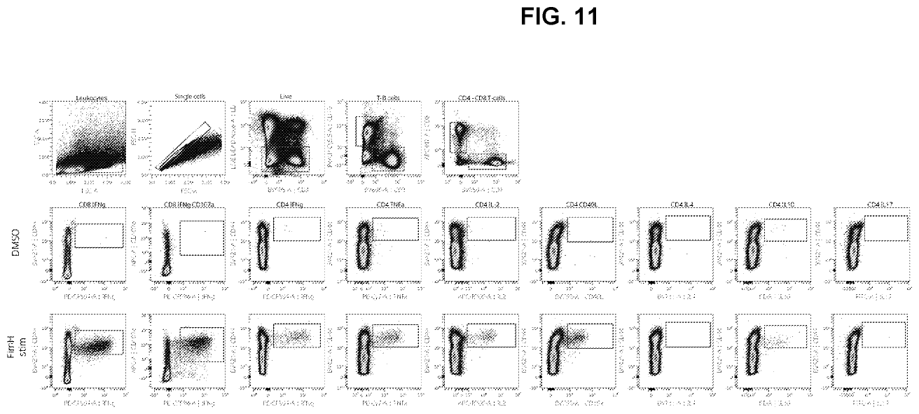

The ability of the FimH vaccine antigens to elicit antibodies capable of preventing the binding of live fimbriated E. coli to immobilized yeast mannan was determined in the neutralization assay. Neutralizing titers from this study are shown in FIG. 10 and Table 12. At least two doses of FimH antigen were required to generate measurable levels of functional antibodies. After two or three vaccinations at either dose level, both modRNA LNPs generated substantially greater functional antibodies than the adjuvanted protein subunit comparator. Titers for the modRNAs peaked after two doses and declined by 4-7 fold after the third dose, an effect that may reflect excessive immunostimulation. Neutralization titers for the two FimH modRNA LNPs were not significantly different from each other at either timepoint or dose level.

TABLE 12
FimH Neutralization Titer Summary

| | Neutralization GMT | | | Responder Rate (%) | | |
|---|---|---|---|---|---|---|
| Antigen | PD1 | PD2 | PD3 | PD1 | PD2 | PD3 |
| FimH_{DSG} 10 μg/5 μg + | 50 | 130 | 192 | 0 | 27 | 47 |
| LiNA-2 | | | | | | |
| Secreted FimH_{DSG}TM | 53 | 26,709 | 7,230 | 5 | 95 | 95 |
| LNP 10 μg | | | | | | |
| Secreted FimH_{DSG}TM | 50 | 7,458 | 1,967 | 0 | 90 | 90 |
| LNP 1 μg | | | | | | |
| FimH_{LD}-TMCtDAFgpi | 102 | 22,783 | 8,340 | 60 | 90 | 95 |
| LNP 10 μg | | | | | | |
| FimH_{LD}-TMCtDAFgpi | 59 | 3,942 | 1,045 | 20 | 70 | 70 |
| LNP 1 μg | | | | | | |

### 3. T-cell Profiling

Intracellular cytokine staining (ICS) and surface detection of activation induced markers (AIM) was used for quantitative determination of FimH-specific T-cell populations in spleens taken from five mice sacrificed after the third vaccine dose. Identification of cytokine or surface marker expression levels in specific cell types was achieved via selective cell gating during flow cytometric analysis FIG. 11. CD107a is a surface marker for immune cell activation and cytotoxic degranulation (Alter G, Malenfant J M, Altfeld M. 2004. CD107a as a functional marker for the identification of natural killer cell activity. J Immunol Methods 294:15-22).

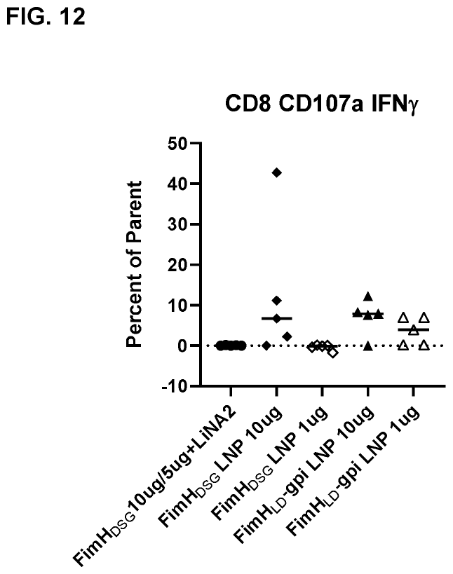

CD8+ T-cell responses in vaccinated mice following FimH peptide activation are shown in FIG. 12. Mice vaccinated with the adjuvanted protein FimH_{DSG} antigen failed to show CD8+ CD107a expression and IFN-γ production upon FimH peptide stimulation. In contrast, four out of five mice vaccinated with 10 μg of the FimH_{DSG} modRNA LNP generated CD107a and IFN-γ (double positive) responses. Similarly, FimH-specific CD8+ T cells were detectable in the majority of mice immunized with 10 μg of mRNA encoding FimH_{LD}-gpi LNP. For the FimH_{LD}-gpi LNP, three of five animals responded even at the 1 μg mRNA dose.

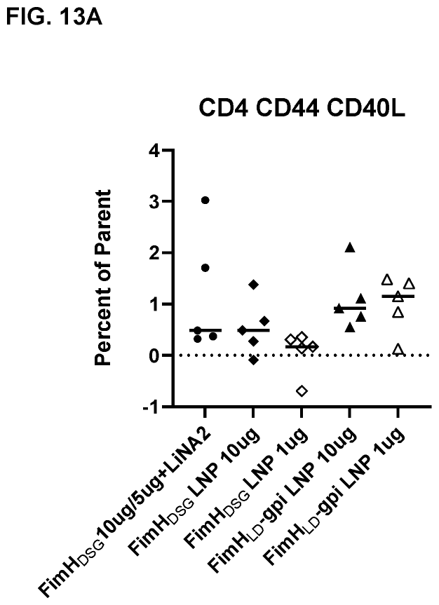

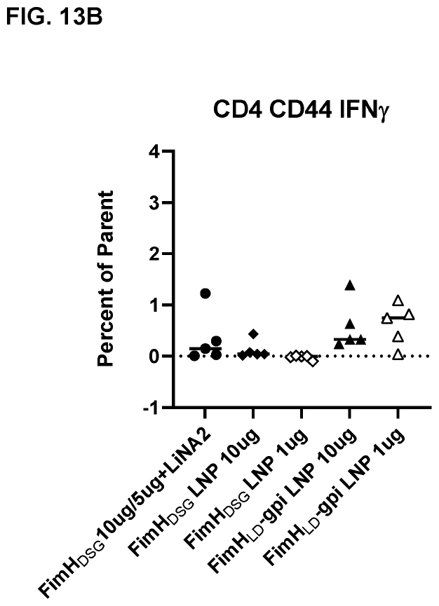

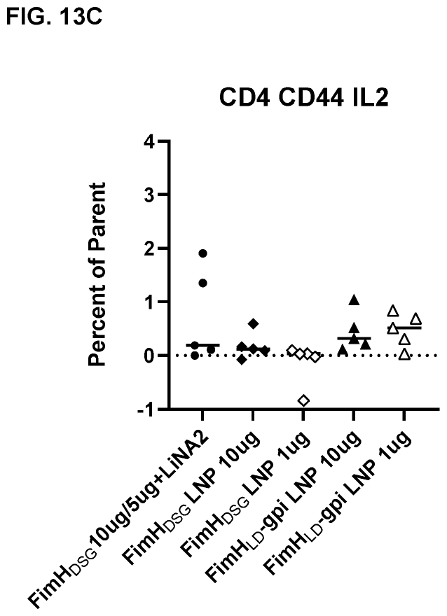

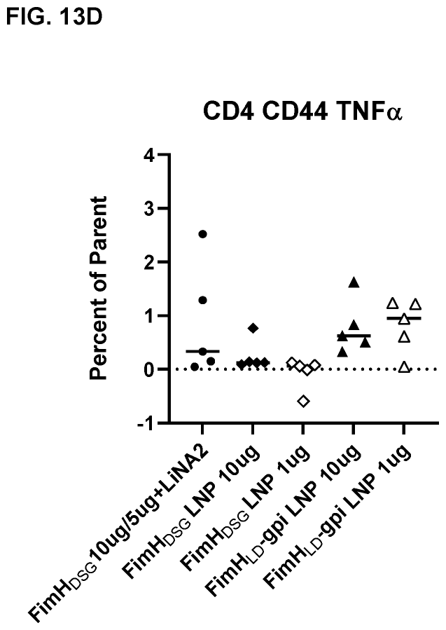

CD4+ T-cell responses in vaccinated mice are shown in FIG. 13A-FIG. 13D Interestingly, FimH peptide pool stimulation demonstrated robust Th1-biased CD4+ T-cell responses in mice vaccinated with mRNA encoding the membrane-targeted FimH_{LD} chimera but to a lower degree in mice vaccinated with the mRNA for the secreted FimH_{DSG} antigen. Mice vaccinated with FimH_{DSG} protein adjuvanted with LiNA-2 also yielded antigen-specific CD4 T-cell responses, although median values of cytokine-positive cells were uniformly lower than responses from mice receiving the membrane-targeted FimH modRNA constructs (at either dose level).

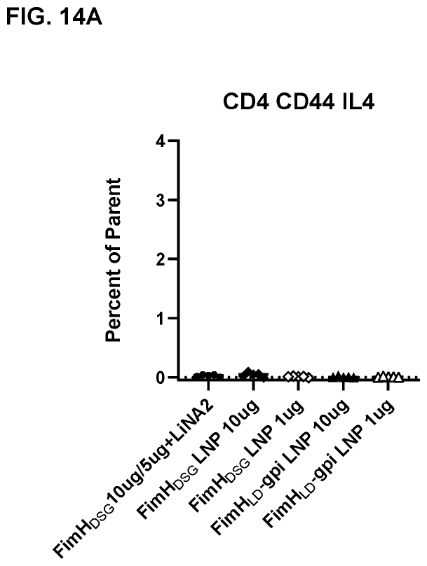

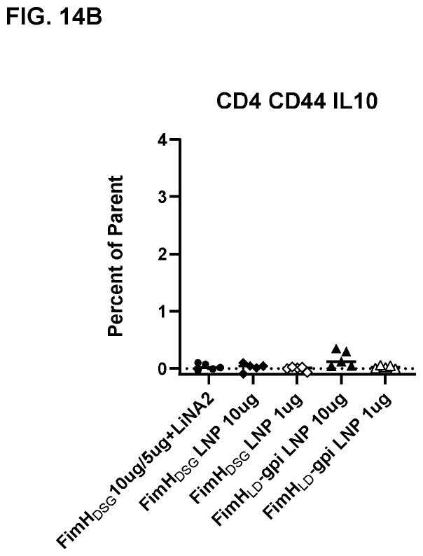

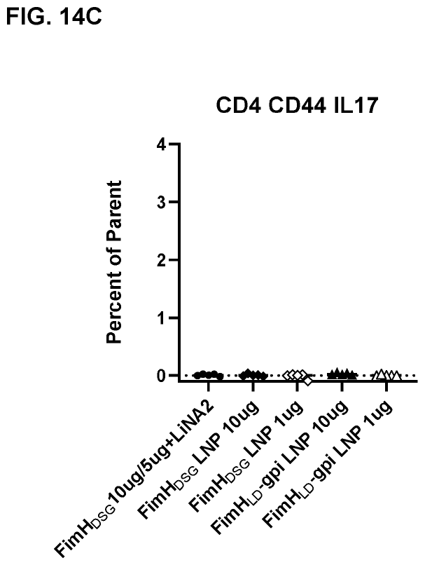

Finally, activation of CD4 T-cell Th2 or Th17 pathway-specific markers were also evaluated and results shown in FIG. 14A-FIG. 14C. In this case, little or no FimH-specific Th2 or Th17 marker expression was detected for CD4 T-cells isolated from mice vaccinated with any of the FimH mRNA antigens. Thus, modRNA-encoded FimH antigens primarily induce CD8+ as well as Th1-biased CD4+ T cell responses but not Th2 or Th17.

In the mouse study described in this Example, exploratory E. coli FimH modRNA LNPs were evaluated for the first time to compare their immunogenicity relative to a recombinant protein full-length FimH_{DSG} subunit antigen adjuvanted with liposomal QS21/MPLA (LiNA-2) described in International Publication No. WO2022/137078, which is hereby incorporated by reference in its entirety. Mice immunized with this benchmark protein formulation elicited substantially weaker serum neutralizing antibodies than the identical protein antigen expressed in mammalian cells from a modRNA. In Example 1 we showed that FimH_{DSG} in its secreted form could be readily detected in culture supernatants of Expi293 suspension cells transfected with FimH_{DSG} mRNA. The modRNA LNP derived from the same plasmid construct generated dose-dependent antigen-specific CD8+ T-cell responses, and robust CD4+ T-cell responses in mice. In contrast the recombinant subunit formulation failed to elicit CD8+ T-cell responses, and generated weaker CD4+ T-cell responses.

An additional modRNA LNP was evaluated with the aim of assessing the impact of the FimH antigen expressed on the mammalian cell surface instead of as a secreted antigen. In this case, the N-terminal lectin binding domain of FimH was fused to a distinct membrane anchoring sequence: 37 C-terminal amino acids of the gpi-anchored human CD55 (decay-accelerating factor or DAF), only nine of which remain after signal cleavage in the endoplasmic reticulum and GPI attachment (Lisanti M P, Caras I W, Davitz M A, Rodriguez-Boulan E. 1989. Journal of Cell Biology 109:2145-2156). Compared with the secreted FimH_{DSG} LNP, this modRNA LNP generated similar levels of FimH neutralizing antibodies but resulted in a stronger upregulation of markers associated with activation of the Th1 pathway (CD40L, IFNγ, IL2, TNFα). None of the antigens tested activated Th2 pathway markers (IL4, IL10) or induced IL17, initiator of the associated pro-inflammatory signaling pathway that links T-cell activation to neutrophil mobilization and activation (Zenobia C, Hajishengallis G. 2015. Periodontology 2000 69:142-159).

These results demonstrate that the modRNA platform is capable of eliciting substantially higher levels of neutralizing antibodies in mice than an adjuvanted recombinant protein subunit antigen described herein.

TABLE 13
SEQUENCES

FimH wild type and mutant sequences
SEQ ID NO: 1 >FimHLD_WT
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 2 >FimHLD_G65A_V27A
FACKTASGTAIPIGGGSANVYVNLAPAVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 3 >FimHLD_F1I
IACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 4 >FimHLD_F1L
LACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 5 >FimHLD_F1V
VACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 6 >FimHLD_F1M
MACKTASGTAIPIGGGSANVYVNLAPVVNVGQNLVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 7 >FimHLD_F1Y
YACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 8 >FimHLD_F1W
WACKTASGTAIPIGGGSANVYVNLAPVVNVGQNLVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 9 >FimHLD_Q133K
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 10 >FimHLD_G15A
FACKTASGTAIPIGAGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 11 >FimHLD_G15P
FACKTASGTAIPIGPGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 12 >FimHLD_G16A
FACKTASGTAIPIGGASANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 13 >FimHLD_G16P
FACKTASGTAIPIGGPSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 14 >FimHLD_G15A_G16A
FACKTASGTAIPIGAASANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 15 >FimHLD_R60P
FACKTASGTAIPIGGGSANVYVNLAPVVNVGQNLVVDLSTQIFCHNDYPETITDYVTLQPGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 16 >FimHLD_G65A
FACKTASGTAIPIGGGSANVYVNLAPVVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 17 >FimHLD_P12C_A18C
FACKTASGTAICIGGGSCNVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 18 >FimHLD_G14C_F144C
FACKTASGTAIPICGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQCVWNIYANNDVVVPTGG
SEQ ID NO: 19 >FimHLD_P26C_V35C
FACKTASGTAIPIGGGSANVYVNLACVVNVGQNLCVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 20 >FimHLD_P26C_V154C
FACKTASGTAIPIGGGSANVYVNLACVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDCVVPTGG
SEQ ID NO: 21 >FimHLD_P26C_V156C
FACKTASGTAIPIGGGSANVYVNLACVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVCPTGG
SEQ ID NO: 22 >FimHLD_V27C_L34C
FACKTASGTAIPIGGGSANVYVNLAPCVNVGQNCVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 23 >FimHLD_V28C_N33C
FACKTASGTAIPIGGGSANVYVNLAPVCNVGQCLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 24 >FimHLD_V28C_P157C
FACKTASGTAIPIGGGSANVYVNLAPVCNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVCTGG
SEQ ID NO: 25 >FimHLD_Q32C_Y108C
FACKTASGTAIPIGGGSANVYVNLAPVVNVGCNLVVDLSTQIFCHNDYPETITDYVTLQRGSAYGGVLS
SFSGTVKYSGSSYPFPTTSETPRVVYNSRTDKPWPVALCLTPVSSAGGVAIKAGSLIAVLILRQTNNYN
SDDFQFVWNIYANNDVVVPTGG
SEQ ID NO: 26 >FimHLD_N33C_L109C
FACKTASGTAIPIGGGSANVYVNLAPVVNVGQCLVVDLSTQIFCHNDYPETITDYVTLQRGSAYGGVLS
SFSGTVKYSGSSYPFPTTSETPRVVYNSRTDKPWPVALYCTPVSSAGGVAIKAGSLIAVLILRQTNNYN
SDDFQFVWNIYANNDVVVPTGG
SEQ ID NO: 27 >FimHLD_N33C_P157C
FACKTASGTAIPIGGGSANVYVNLAPVVNVGQCLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVCTGG
SEQ ID NO: 28 >FimHLD_V35C_L107C
FACKTASGTAIPIGGGSANVYVNLAPVVNVGQNLCVDLSTQIFCHNDYPETITDYVTLQRGSAYGGVLS
SFSGTVKYSGSSYPFPTTSETPRVVYNSRTDKPWPVACYLTPVSSAGGVAIKAGSLIAVLILRQTNNYN
SDDFQFVWNIYANNDVVVPTGG
SEQ ID NO: 29 >FimHLD_V35C_L109C
FACKTASGTAIPIGGGSANVYVNLAPVVNVGQNLCVDLSTQIFCHNDYPETITDYVTLQRGSAYGGVLS
SFSGTVKYSGSSYPFPTTSETPRVVYNSRTDKPWPVALYCTPVSSAGGVAIKAGSLIAVLILRQTNNYN
SDDFQFVWNIYANNDVVVPTGG
SEQ ID NO: 30 >FimHLD_S62C_T86C
FACKTASGTAIPIGGGSANVYVNLAPVVNVGQNLVVDLSTQIFCHNDYPETITDYVTLQRGCAYGGVLS
SFSGTVKYSGSSYPFPCTSETPRVVYNSRTDKPWPVALYLTPVSSAGGVAIKAGSLIAVLILRQTNNYN
SDDFQFVWNIYANNDVVVPTGG
SEQ ID NO: 31 >FimHLD_S62C_L129C
FACKTASGTAIPIGGGSANVYVNLAPVVNVGQNLVVDLSTQIFCHNDYPETITDYVTLQRGCAYGGVLS
SFSGTVKYSGSSYPFPTTSETPRVVYNSRTDKPWPVALYLTPVSSAGGVAIKAGSLIAVCILRQTNNYN
SDDFQFVWNIYANNDVVVPTGG
SEQ ID NO: 32 >FimHLD_Y64C_L68C
FACKTASGTAIPIGGGSANVYVNLAPVVNVGQNLVVDLSTQIFCHNDYPETITDYVTLQRGSACGGVC
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 33 >FimHLD_Y64C_A127C
FACKTASGTAIPIGGGSANVYVNLAPVVNVGQNLVVDLSTQIFCHNDYPETITDYVTLQRGSACGGVLS
SFSGTVKYSGSSYPFPTTSETPRVVYNSRTDKPWPVALYLTPVSSAGGVAIKAGSLICVLILRQTNNYN
SDDFQFVWNIYANNDVVVPTGG
SEQ ID NO: 34 >FimHLD_L68C_F71C
FACKTASGTAIPIGGGSANVYVNLAPVVNVGQNLVVDLSTQIFCHNDYPETITDYVTLQRGSAYGGVCS
SCSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 35 >FimHLD_V112C_T158C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPCSSAGGVAIKAGSLIAVLILRQTNNYN
SDDFQFVWNIYANNDVVVPCGG
SEQ ID NO: 36 >FimHLD_S113C_G116C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVCSACGVAIKAGSLIAVLILRQTNNYN
SDDFQFVWNIYANNDVVVPTGG
SEQ ID NO: 37 >FimHLD_S113C_T158C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVCSAGGVAIKAGSLIAVLILRQTNNYN
SDDFQFVWNIYANNDVVVPCGG
SEQ ID NO: 38 >FimHLD_V118C_V156C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGCAIKAGSLIAVLILRQTNNYN
SDDFQFVWNIYANNDVVCPTGG
SEQ ID NO: 39 >FimHLD_A119C_V155C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVCIKAGSLIAVLILRQTNNYN
SDDFQFVWNIYANNDVCVPTGG
SEQ ID NO: 40 >FimHLD_L34N_V27A
FACKTASGTAIPIGGGSANVYVNLAPAVNVGQNNVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 41 >FimHLD_L34S_V27A
FACKTASGTAIPIGGGSANVYVNLAPAVNVGQNSVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 42 >FimHLD_L34T_V27A
FACKTASGTAIPIGGGSANVYVNLAPAVNVGQNTVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 43 >FimHLD_A119N_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVNIKAGSLIAVLILRQTNNYN
SDDFQFVWNIYANNDVVVPTGG
SEQ ID NO: 44 >FimHLD_A119S_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVSIKAGSLIAVLILRQTNNYN
SDDFQFVWNIYANNDVVVPTGG
SEQ ID NO: 45 >FimHLD_A119T_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVTIKAGSLIAVLILRQTNNYN
SDDFQFVWNIYANNDVVVPTGG
SEQ ID NO: 46 >FimH-DSG_A115V
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSVGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 47 >FimH-DSG_V163I
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDISARDVTVTLPDYPGSVPIPLTVYCAKSQNLGYYLSGTTADAGN
SIFTNTASFSPAQGVGVQLTRQGTIIPANNTVSLGAVGTSAVSLGLTANYARTGGQVTAGNVQSIIGVT
FVYQGGSSGGGADVTITVNGKVVAK
SEQ ID NO: 48 >FimH-DSG_V185I
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTIYCAKSQNLGYYLSGTTADAGN
SIFTNTASFSPAQGVGVQLTRQGTIIPANNTVSLGAVGTSAVSLGLTANYARTGGQVTAGNVQSIIGVT
FVYQGGSSGGGADVTITVNGKVVAK
SEQ ID NO: 49 >FimH-DSG_DSG_V3I
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADITITVNGKVVAK
SEQ ID NO: 50 >FimHLD_G15A_V27A
FACKTASGTAIPIGAGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 51 >FimHLD_G16A_V27A
FACKTASGTAIPIGGASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 52 >FimHLD_G15P_V27A
FACKTASGTAIP|GPGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 53 >FimHLD_G16P_V27A
FACKTASGTAIPIGGPSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 54 >FimHLD_G15A_G16A_V27A
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 55 >FimHLD_V27A_R60P
FACKTASGTAIPIGGGSANVYVNLAPAVNVGQNLVVDLSTQIFCHNDYPETITDYVTLQPGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 56 >FimHLD_G65A_V27A
FACKTASGTAIPIGGGSANVYVNLAPAVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 57 >FimHLD_V27A_Q133K
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 58 >FimHLD_G15A_G16A_V27A_Q133K
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 59 >FimH-DSG_WT
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 60 >FimH-DSG_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 61 >FimH-DSG_G15A_V27A
FACKTASGTAIPIGAGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 62 >FimH-DSG_G15A_G16A_V27A
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 63 >FimH-DSG_V27A_Q133K
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 64 >FimH-DSG_G15A_G16A_V27A_Q133K
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 65 >mouse Ig Kappa signal peptide
ETDTLLLWVLLLWVPGSTG

1. A ribonucleic acid polynucleotide (RNA) molecule comprising at least one open reading frame (ORF) encoding a FimH antigenic polypeptide.

2. The RNA e of claim 1, wherein the FimH antigenic polypeptide is a full-length, truncated, fragment or variant thereof.

3. The RNA molecule of any one of claims 1 to 2, wherein the FimH antigenic polypeptide comprises at least one mutation.

4. The RNA molecule of any one of claims 1 to 3, wherein the FimH antigenic polypeptide comprises an amino acid of Table 13, including but not limited to any of SEQ ID NO: 1 to 64.

5. The RNA molecule of any one of claims 1-4, wherein the FimH antigenic polypeptide comprises FimH-DSG (SEQ ID NO: 59), FimH-DSG triple mutant (G15A, G16A, V27A) (SEQ ID NO: 62), FimHLD triple mutant (G15A, G16A, V27A) (SEQ ID NO: 54), or an immunogenic fragment thereof.

6. The RNA molecule of any one of claims 1 to 5, wherein the FimH polypeptide has at least 90%, 95, 96%, 97%, 98% or 99% identity to the amino acid sequence selected from SEQ ID NO: 1 to 64.

7. The RNA molecule of any one of claims 1-6, wherein the RNA is fused to a C-terminal membrane targeting domain.

8. The RNA molecule of claim 7, wherein the RNA molecule and the C-terminal membrane targeting domain are separated by a linker.

9. The RNA molecule of claim 8, wherein the linker has the amino acid sequence GGSSGGG (SEQ ID NO: 74).

10. The RNA molecule of any of claims 7-9, wherein the C-terminal membrane targeting domain is derived from a viral glycoprotein.

11. The RNA molecule of claim 10, wherein the viral glycoprotein is selected from the group consisting of HSV gD, SARS-CoV2 Spike protein, and human DAFgpi.

12. The RNA of claim 9, wherein the C-terminal membrane targeting domain is an E. coli G-peptide.

13. The RNA molecule of any of claims 1-12, wherein the open reading frame is codon-optimized.

14. The RNA molecule of claim 11, wherein the FimH antigenic polypeptide comprises an amino acid of Table 1, including but not limited to any of SEQ ID NO: 82, SEQ ID NO: 83, and SEQ ID NO: 84.

15. The RNA molecule of claim 14, wherein the FimH antigenic polypeptide comprises an amino acid having SEQ ID NO: 84.

16. The RNA molecule of claim 12, wherein the FimH antigenic polypeptide comprises an amino acid having SEQ ID NO: 85.

17. The RNA molecule of any one of claims 1 to 16, wherein the open reading frame is transcribed from a nucleic acid sequence of Table 2, including but not limited to any of SEQ ID NO: 66, SEQ ID NO: 68, SEQ ID NO: 70 or SEQ ID NO: 72.

18. The RNA molecule of any one of claims 1 to 17, wherein the open reading frame comprises a nucleic acid sequence of Table 3, including but not limited to any of SEQ ID NO: 82 to 85.

19. The RNA of claim 18, wherein each uridine of any of SEQ ID NO: 82 to 85 is replaced by 1-methyl-3′-pseudouridylyl (4P).

20. The RNA molecule of any one of claims 1 to 19, further comprising a 5′ untranslated region (5′ UTR).

21. The RNA molecule of claim 21, wherein the 5′ UTR comprises nucleotides having SEQ ID NO: 75.

22. The RNA molecule any one of claims 1 to 21, further comprising a 3′ untranslated region (3′ UTR).

23. The RNA molecule of claim 22, wherein the 3′ UTR comprises nucleotides having SEQ ID NO: 76.

24. The RNA molecule of any one of claims 1 to 23, wherein the RNA molecule comprises a 5′ cap moiety.

25. The RNA molecule of claim 24, wherein the 5′ cap moiety is m7G(5′)ppp(5′)(2′OMeA)pG.

26. The RNA molecule of any one of claims 1 to 25, further comprising a 3′ poly-A tail.

27. The RNA of claim 26, wherein the poly A tail comprises a sequence having SEQ ID NO: 86.

28. The RNA molecule of any one of claims 1 to 27, wherein the RNA molecule comprises a 5′ UTR and 3′ UTR.

29. The RNA molecule of any one of claims 1 to 28, wherein the RNA molecule comprises a 5′ cap, 5′ UTR, and 3′ UTR.

30. The RNA molecule of any one of claims 1 to 29, wherein the RNA molecule comprises a 5′ cap, 5′ UTR, 3′ UTR, and poly-A tail.

31. The RNA molecule of any of claims 1 to 30, wherein the RNA molecule comprises stabilized RNA.

32. The RNA molecule of any one of claims 1 to 31, wherein the RNA comprises at least one modified nucleotide.

33. The RNA molecule of claim 32, wherein the modified nucleotide is pseudouridine, 1-methyl-3′-pseudouridylyl, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, or 5-methoxyuridine OR 2′-O-methyl uridine.

34. The RNA molecule of claim 33, wherein the modified nucleotide is 1-methyl-3′-pseudouridylyl (4P).

35. The RNA molecule of any one of claims 1 to 25, wherein the RNA is mRNA.

36. A composition comprising the RNA molecule of any one of claims 1 to 35, wherein the RNA molecule is formulated in a lipid nanoparticle (RNA-LNP).

37. The composition of claim 36, wherein lipid nanoparticle comprises at least one of a cationic lipid, a PEG-lipid, a neutral lipid, and a steroid or steroid analog.

38. The composition of claim 36 or 37, wherein the lipid nanoparticle comprises a cationic lipid.

39. The composition of claim 38, wherein the cationic lipid is (4-hydroxybutyl)azanediyl)bis(hexane-6,1-diyl)bis(2-hexyldecanoate) (ALC-0315).

40. The composition of any one of claims 36 to 39, wherein the lipid nanoparticle comprises a PEG-lipid.

41. The composition of claim 40, wherein the PEG-lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG,N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate.

42. The composition of claim 40 or 41, wherein the PEG-lipid is 2-[(polyethylene glycol)-2000]-N,N-ditetradecylacetamide (ALC-0159).

43. The composition of any one of claims 36 to 42, wherein the lipid nanoparticle comprises a neutral lipid.

44. The composition of claim 43, wherein the neutral lipid is distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).

45. The composition of claim 43 or 44, wherein the neutral lipid is 1,2-distearoyl-sn-glycero-3-phosphocholine (DSPC).

46. The composition of any one of claims 36 to 45, wherein the lipid nanoparticle comprises a steroid or steroid analog.

47. The composition of claim 46, wherein the steroid or steroid analog is cholesterol.

48. The composition of any one of claims 36 to 47, wherein lipid nanoparticle wherein has a mean diameter of about 1 to about 500 nm.

49. The composition of any one of claims 36 to 48, wherein the composition is a vaccine.

50. The composition of any one of claims 36 to 49, wherein the lipid nanoparticle size is at least 40 nm.

51. The composition of any one of claims 36 to 49, wherein the lipid nanoparticle size is at most 180 nm.

52. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of the RNA molecule, RNA-LNP and/or vaccine of any one of claims 1 to 51.

53. The method of claim 52, wherein the subject is at risk of developing a urinary tract infection.

54. The method of claim 52, wherein the subject is at risk of developing bacteremia.

55. The method of claim 52, wherein the subject is at risk of developing urosepsis.

56. The method of claim 52, wherein the subject is at risk of developing cystitis.

57. Use of the RNA molecule, RNA-LNP and/or composition of any one of claims 1 to 56 in the manufacture of a medicament for use in (i) inducing an immune response in a subject against extra-intestinal pathogenic E. coli, or (ii) inducing the production of opsonophagocytic and/or neutralizing antibodies in a subject that are specific to extra-intestinal pathogenic E. coli.

58. The use of claim 57, wherein the infection, disease or condition is a urinary tract infection.

59. The use of claim 57, wherein the subject is at risk of developing bacteremia.

60. The use of claim 57, wherein the subject is at risk of developing sepsis.

61. The use of claim 57, wherein the subject is at risk of developing cystitis.

62. The method or use of any one of claims 52 to 61, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.

63. The method or use of any one of claims 52 to 61, wherein the subject is about 50 years of age or older.

64. The method or use of any one of claims 52 to 61, wherein the subject is a pregnant woman.

65. The method or use of any one of claims 52 to 64, wherein the RNA molecule or composition is administered as a vaccine.

66. The method or use of any one of claims 52 to 65, wherein the RNA molecule or composition is administered by intradermal or intramuscular injection.
